(19) United States
(12) Patent Application Publication
(10) Pub. No.: US 2024/0207552 A1
(43) Pub. Date: Jun. 27, 2024

(54) DEVICE, PROCESS AND COMPUTER PROGRAM FOR DETERMINING SITUATIONS OF A PATIENT

(71) Applicant: Drägerwerk AG & Co. KGaA, Lübeck (DE)

(72) Inventors: Marcus EGER, Lübeck (DE); Frank FRANZ, Stockelsdorf (DE)

(21) Appl. No.: 17/919,064

(22) PCT Filed: Feb. 28, 2022
(86) PCT No.: PCT/DE2022/100160

§ 371 Date: Oct. 14, 2022

(30) Foreign Application Priority Data
Jun. 18, 2021 (DE) 10 2021 115 865.6
Jun. 18, 2021 (DE) 10 2021 115 866.4
Jun. 18, 2021 (DE) 10 2021 115 867.2

(51) Int. Cl. A61M 16/00

(52) U.S. Cl. CPC A61M 16/026; A61M 16/0003; A61M 2205/3303; A61M 2205/3327; A61M 2205/502; A61M 2210/1014; A61M 2210/1025; A61M 2230/08; A61M 2230/40

(57) ABSTRACT

A device, a process and a computer program, pertaining to a determination of situations during breathing or during a ventilation are described. Concepts for obtaining, detecting or determining information are described. The information, for example, is information concerning the respiratory muscles of the patient, concerning a load-bearing capacity of a patient, concerning a need for breathing assistance and also concerning the possibilities of adequately assisting the breathing by stimulation are very valuable in the treatment and therapy of living beings or patients.

### CROSS REFERENCE TO RELATED APPLICATIONS

This application is a United States National Phase Application of International Application PCT/DE2022/100160, filed Feb. 28, 2022, and claims the benefit of priority under 35 U.S.C. § 119 of German Application 10 2021 115 866.4, filed Jun. 18, 2021, German Application 10 2021 115 867.2, filed Jun. 18, 2021, and German Application 10 2021 115 865.6, filed Jun. 18, 2021, the entire contents of which are incorporated herein by reference.

### TECHNICAL FIELD

Situations of a patient, especially situations during the ventilation of a patient or of an assist of the ventilation or breathing of a patient, require information about the patient, for example, concerning the respiratory muscles of the patient, concerning the load-bearing capacity of the patient, concerning the need of the patient for breathing assistance and also concerning the possibilities of an adequate stimulative assistance of the patient's breathing. The obtaining of information concerning such information with concepts for determining states of a patient in the area of breathing and/or ventilation form the basis for the objects described within the framework of the following description and solutions for accomplishing them.

The present invention pertains to a device, to a process and to a computer program for determining a state of the respiratory muscles of a patient, especially but not exclusively to a concept for determining at least one state parameter of the respiratory muscles of a patient on the basis of an analysis of an activation signal as a response to a stimulation of the respiratory muscles.

The present invention pertains, moreover, to a concept for determining a load-bearing capacity (load capacity) and an indicator of a component of the ventilation, which is attributed by the patient themself (a measure of the patient-portion of ventilation) and for taking into consideration the load-bearing capacity and the indicator during the assistance of the ventilation. In another aspect, the present invention pertains to a ventilation system, to a device, to a process and to a computer program for ventilating a patient.

The present invention pertains, moreover, to a concept for determining an indicator of a breathing assistance of a patient based on a desired respiratory muscle activation and on an actual respiratory muscle activation. In another aspect, the present invention pertains to a device, to a process and to a computer program for a component of a ventilation system for the breathing assistance of a patient, to a ventilator, to a stimulator, to a sensor unit, to a process and to a computer program for a ventilator, to a stimulator and to a sensor unit, to a ventilation system, to a process and to a computer program for a ventilation system.

The present invention pertains, moreover, to a concept (device, system and process) for the stimulative ventilatory assist of a patient, synchronized with a spontaneously generated respiratory muscle activity of the patient. In another aspect, the present invention pertains to a ventilation system, to a device, to a process and to a computer program for the stimulative ventilatory assist of a patient.

All these above-mentioned concepts and/or aspects of inventions are combined under the effort to improve the situation of a patient during ventilation by an adequate to the actual situation of load, load-bearing capacity and the need of the patient for breathing assistance, being provided. The provision may be accomplished in the form of concepts and actions in respect to the performance of automated ventilation and/or the performance of stimulations for activating breathing efforts of the patient. The performance of automated ventilation may be carried out as mandatory ventilation, triggered mandatory ventilation, and assisting ventilation.

### TECHNICAL BACKGROUND

The maintenance and recovery of spontaneous breathing has had a high priority in intensive care for a long time. In case spontaneous breathing is not possible for the patient, a lung-protective ventilation is applied, which shall damage the lung tissue as little as possible. The protection of the respiratory muscles, especially of the diaphragm, has become a priority only recently. State of the art of the present invention can be found, for example, in the following documents: U.S. Pat. No. 5,820,560 B1; U.S. Pat. No. 7,021,310 B1; WO 2019 15 48 34 A1; WO 2019 15 48 37 A1; WO 2019 154 839 A1; WO 20 2007 92 66 A1; WO 2020 188 069 A1; DE 10 2019 006 480 A1; US 2017 0252558 A1; DE 10 2019 006 480 A1; US 2015 0366480 A1; US 2012 0103334 A1; DE 10 2019 007 717 B3; DE 10 2020 000 014 A1; DE 10 2007 062 214 B3; WO 2018 143 844 A1; DE 10 2015 011 390 A1; DE 10 2021 115 865 A1; DE 10 2020 123 138 A1; and DE 10 2007 052 897 B4.

The sources mentioned below provide additional information on the technical, medical technical, medical as well as clinical background. The following list comprises an exemplary selection of documents and publications on the detection and processing of different measured signals or signals in/at the human body and on the use thereof within the framework of ventilation, ventilation control as well as in the setting of ventilation with breathing stimulation.
Walker, D. J.: “Prediction of the Esophageal Pressure by the Mouth Closing Pressure Following Magnetic Stimulation of the Phrenic Nerve,” Dissertation, University of Freiburg, 2006.
Kahl, L. et al.: “Comparison of Algorithms to Quantify Muscle Fatigue in Upper Limb Muscles Based on sEMG Signals,” Medical Engineering & Physics, 2016.
Jansen, D. et al.: “Estimation of the Diaphragm Neuromuscular Efficiency Index in Mechanically Ventilated Critically Ill Patients,” Critical Care, 2018.
Liu, L. et al.: “Neuroventilatory Efficiency and Extubation Readiness in Critically Ill Patients,” Critical Care, 2012.
Cattapan, S. E. et al.: “Can Diaphragmatic Contractility Be Assessed by Airway Twitch Pressure in Mechanically Ventilated Patients?,” Thorax, 2003.
Younes, M. et al.: A Method for Monitoring and Improving Patient Ventilator Interaction,” Intensive Care Med., 2007.
Blanch Lluis et al.: “Measurement of Air Trapping, Intrinsic Positive End-Expiratory Pressure and Dynamic Hyperinflation in Mechanically Ventilated Patients,” Respiratory Care, 2005.
Purro, A. et al.: “Static Intrinsic PEEP in COPD Patients During Spontaneous Breathing,” AJRCCM, 1988.
Bernardi, E. et al.: “A New Ultrasound Method for Estimating Dynamic Intrinsic Positive Airway Pressure: A Prospective Clinical Trial,” AJRCCM, 2018.
Pisani, L. et al.: “Noninvasive Detection of Positive End-Expiratory Pressure in COPD Patients Recovering from Acute Respiratory Failure,” European Respiratory Journal, 2016.
Bellani, G. et al.: “Clinical Assessment of Auto-positive End-Expiratory Pressure by Diaphragmatic Electrical Activity During Pressure Support and Neurally Adjusted Ventilatory Assist,” Anesthesiology,” 2014.
Younes, M.: “Dynamic Intrinsic PEEP (PEEPi,dyn): Is It Worth Saving?,” AJRCCM, 2000.
Shalaby, R. E.-S.: “Development of an Electromyography Detection System for the Control of Functional Electrical Stimulation in Neurological Rehabilitation,” Dissertation, 2011.
Younes, M. et al.: “A Method for Monitoring and Improving Patient Ventilator Interaction,” Intensive Care Med.,” (2007).
Putensen, C. et al.: “Long-Term Effects of Spontaneous Breathing During Ventilatory Support in Patients with Acute Lung Injury,” AM Journal Respir. Crit. Care Med.,” 2001.
Otis, A. B. et al.: “Mechanics of Breathing in Man,” J. Appl. Physiol., 1950.
Osuagwu, B. A. C. et al.: “Active Proportional Electromyogram Controlled Functional Electrical Stimulation System, Nature, 2020.
Virtanen, J. et al.: “Instrumentation for the Measurement of Electric Brain Responses to Transcranial Magnetic Stimulation,” Med. Biol. Eng. Comput., 1999.

Reference will be made at times to these and other documents in connection with special aspects of the present invention in the course of the description. Instead of extensive citations, reference will be made at times to the publications listed as references [E1] through [E38] in a Table 4 included following the description of the figures and preferred embodiments. To avoid a lack of clarity based on linguistic wordings, some references should be made at the beginning of the application for the understanding as well as some explanations shall be given concerning the use of terms. In the description and/or in the patent claims, the wordings in the verbal form, nominalized verbal form, which are used in the process and also due to embodiments of a control unit in embodiments of the devices, for example and especially “a determination,” “a high-pass filtering,” “a determining,” “a stimulating,” “a performing,” “a transforming,” “an outputting,” “a detecting,” “a setting” are used within the framework of the present invention or inventions with the same meaning as wordings with nouns in the nominalized form, for example and especially “a determination,” “a high-pass filtration,” “a determination,” “a performance,” “an output,” “a detection,” “a setting,” “a stimulation.” Equivalent and similarly acting meanings are obtained in respect to the disclosure of the invention or inventions for the wordings with nouns, nouns, nominalized verbs and verbs, so that reference is or can also always mutually be made for features and details between the verbal form and the nominalized form concerning the disclosure. Wordings with “performances of determinations, high-pass filtrations, stimulations, stimulatings, determinings, detectings, inputs, outputs, transformations, etc.” shall also be considered to be included in the equivalent and similarly acting meanings. Features and details that are described within the framework of the present inventions in connection with devices and embodiments of devices also apply, of course, in connection with the processes described within the framework of the present invention and of the embodiments thereof as well as vice versa, so that reference is and can always mutually be made to the individual aspects of the present invention concerning the disclosure. Furthermore, wordings, which are used to designate and assign parts, units, elements or modules in or at devices or systems may always be used synonymously for one another and correspond to one another in technical details, e.g., “control unit” and “control module.” A similar statement may also be made, for example, in case of the designation “stimulator;” A stimulator may also be called “a stimulation device” or a “device for stimulation.” A similar statement may also be made for the designation “ventilator;” A ventilator may also be called a ventilation unit or a unit for ventilation.

DE 10 2019 006 480 A1 [E14] describes a process, which makes it possible to estimate components of the work of breathing by means of electromyography of the respiratory muscles. Electromyography (EMG) is a neurological examination for living beings, in which the natural electrical activity of a muscle is measured. Electromyography (EMG) makes it possible to determine the force with which a muscle is exerted. Measurements on superficial muscles are also called sEMG. Electrical Impedance Myography (EIM) is a non-invasive technique for assessing the health of the muscles, wherein the properties of individual muscles or muscle groups or even the composition of muscles and the microscopic structures of muscles can also be examined by means of electrical impedance measurements. Myomechanography (MMG) is a process for detecting elastic, viscous and plastic qualities of muscles. The parameter Pmus represents a variable derived from an EMG signal detected by measurement (electromyogram), from an sEMG signal (Surface Electro-Myogram), from an EIM signal (Electrical Impedance Myogram) or from an MMG signal (mechanomyogram). The parameter Pmus indicates here a pressure level, which has been elicited on the basis of a muscle breathing effort of a patient. The cause of the muscle breathing effort may be initiated by the patient themself in the form of a spontaneous breathing activity and/or it may have been elicited by means of an external, for example, electrical, magnetic or electromagnetic stimulation. The muscle breathing effort may be derived, on the one hand, indirectly from electrical, electromagnetic or magnetic signals. Muscle breathing efforts may also be detected directly by measurement as a pressure difference against a reference pressure, for example, by means of a pressure measurement at the thorax of a patient. The ambient pressure may be selected here as a reference pressure or it is also possible to select a pressure level, which is provided by a ventilator. Pressure levels typically provided by ventilators are, for example, an inspiratory pressure level, usually designated as an inspiratory pressure or inspiration pressure Pinsp, as well as, for example, an expiratory pressure level, usually called an expiratory pressure or expiration pressure Pexp; the so-called PEEP (positive end-expiratory pressure), which describes a pressure level which can be detected by measurement at the end of the exhalation in the airways of the patient as a pressure difference against the ambient pressure, represents a special case of an expiratory pressure level. Both the inspiratory pressure Pinsp and the expiratory pressure Pexp are detected as a pressure difference against the ambient pressure and are usually stated in the unit mbar. Such a parameter Pmus may also be called respiratory muscle pressure Pmus. The terms “muscle airway pressure,” “respiratory muscle pressure” are used in the description and/or in the patent claims within the framework of the present invention or inventions with wordings such as “parameter Pmus,” “pressure parameter Pmus,” “pressure parameter or parameter P, Pmus, which indicates a pressure which has been caused by a muscle breathing effort of a patient,” “a breathing pressure Pmus generated by the muscles of a patient” as terms having the same meaning and producing the identical effect, so that reference can mutually be made concerning the term. The parameter Flowmus represents a variable derived from the parameter Pmus. Higher frequencies in the signal curve of the parameter Pmus can provide an indicator of muscle-related components, flow direction and reversal of the flow direction of the airway flow. The high-pass filtering of the parameter Pmus can be used to determine the parameter Flowmus. Flow rates that flow into the patient as volumes during inhalation phases, i.e., which are inhaled, or which flow out of the patient during exhalation phases, i.e., which are exhaled, are designated by the term airway flow. The parameter Flowmus indicates here a flow rate with a flow direction, and the cause of the flow is based on a muscle breathing effort of a patient. The cause of the muscle breathing effort may be initiated by the patient themself in the form of a spontaneous breathing activity and/or elicited by means of an external, for example, electrical, magnetic or electromagnetic stimulation. Such a parameter Flowmus may also be called a muscle airway flow or even as a respiratory muscle flow Flowmus. The signal processing with high-pass signal filtering of the signal curve of the parameter Pmus makes it possible to determine and find muscle-related changes in the phases of breathing and in times in the parameter Flowmus, at which a reversal of the sign of the parameter Flowmus takes place. The reversal of the sign of the parameter Flowmus indicates here times at which a change of the breathing phases between inhalation phases (inhalation) and exhalation phases (exhalation), which are based on muscle breathing efforts of the patient, take place.

The terms “muscle airway flow,” “respiratory muscle flow” with wordings such as “parameter Flowmus,” “flow parameter Flowmus,” “flow parameter or parameter Flow, Flowmus, which indicates a pressure which has been caused by a muscle breathing effort of a patient,” and “a flow rate Flowmus brought about by the muscles of a patient” are used in an equivalent manner or in a manner producing the identical effect in the description and/or in the patent claims within the framework of the present invention or inventions, so that reference can mutually be made concerning the term. The following list is used to clarify some terms used within the framework of this application:
An airway pressure is defined in the sense of the present invention as a pressure or as a pressure level—mostly and usually above the ambient pressure—in the airways, in the lungs, in the trachea of a living being.
A muscle airway pressure or a respiratory muscle pressure is defined in the sense of the present invention as a breathing pressure brought about by muscles, as a component of the airway pressure, especially on the basis of—spontaneous or stimulated—muscle activity of the respiratory muscles and/or muscle activity of the auxiliary respiratory muscles and/or muscle activity of the auxiliary respiratory muscles of the living being.
An airway flow or a breathing gas flow is defined in the sense of the present invention as any movement of quantities of breathing gas as a quantity of inhaled gases to and into a living being, as well as out of and from a living being as a quantity of exhaled gases.
A muscle airway flow (or a respiratory muscle flow) is defined in the sense of the present invention as any movement of quantities of breathing gas out of and from a living being as a quantity of exhaled gases on the basis of—spontaneous or stimulated—muscle activity of the respiratory muscles and/or of muscle activity of the auxiliary respiratory muscles of the living being.

The terms muscle airway pressure and respiratory muscle pressure are used synonymously within the framework of the present invention. The terms muscle airway flow and respiratory muscle flow are used synonymously within the framework of the present invention.

The respiratory muscles comprise the main muscle acting during inhalation, the diaphragm, and the auxiliary muscles. These include, among other things, the external intercostal muscles (acting during inhalation) and the internal intercostal muscles (acting during exhalation) and the abdominal muscles acting during exhalation. It was thus determined that the diaphragm becomes atrophied due to long ventilation times and excessive assistance of the spontaneous breathing and this requires an extensive weaning. On the other hand, the respiratory muscles may be exhausted and damaged (fatigue) because of increased respiratory load (obstruction, restriction). Some patients tend to make excessive intrinsic breathing efforts in certain situations, which may in turn damage the lungs. How the respiratory muscles can be stimulated is described in the state of the art. All muscles can be stimulated directly by activation of the muscle fibers or of the supplying efferent nerves. For example, the muscle fibers of the diaphragm can be stimulated directly transcutaneously. As an alternative, the phrenic nerve, which is responsible for the contraction of the diaphragm, can be stimulated. Activation of the muscles and contraction take place in both cases. The goal of these processes is to improve the weaning, to promote the removal of secretion and possibly also to avoid ventilation or breathing assistance. Unlike during ventilation or assisted spontaneous breathing, administration of a breathing gas is not necessary in this case.

A flow and pressure sensor is used in US 20170252558A1 as well as in Cattapan, S. E. et al.: “Can Diaphragmatic Contractility Be Assessed by Airway Twitch Pressure in Mechanically Ventilated Patients?,” Thorax, 2003 to determine the work of breathing of the patient and to adapt the stimulation such that a time corridor is obtained. However, there is no information technological connection to a ventilator. Parameters of the mechanics of breathing must be taken from the graphic user interface of the ventilator and be entered manually into the separate stimulator. It is, furthermore, not possible to rely on the indicated values of the mechanics of breathing, which are calculated from pneumatic signals, in conventional ventilators as long as the patient is breathing spontaneously to a great degree, i.e., the determined indicator of the work of breathing is only a rough estimate.

Accordingly, there is no method so far that can adequately adjust and coordinate the ventilation and the stimulation especially in view to the work of breathing to be performed. It is self-evident and known, for example, from WO 2019154834, WO 2019154837, WO 19154839, WO 2020079266, and US 2017/0252558A1, that stimulation and ventilation must be coordinated in their basic mechanism. However, there is no method so far that can predefine the degree of assistance and of stimulation, e.g., depending on a therapy goal. The minute volume necessary for the patient as well as threshold values and limits of the mechanical pressure assistance (triggers, pressures, frequencies) are usually set. The component to be contributed by the patient (a patient's own efforts) can hardly be set, because there is no sufficiently precise possibility so far for splitting the work of breathing between machine and patient. A process in which mechanics of breathing parameters of the ventilator used are transferred manually and are then used in the stimulator is practically unaffordable: The parameters change, e.g., after repositioning of the patient. Consequently, they would have to be entered repeatedly. In addition, they are very inaccurate as long as only pneumatic signals are used in the ventilator for determining the parameters. Consequently, the actual contribution of the patient is hardly known, since it would be necessary for this to know the contribution of the patient to the driving pressure (Pmus) or breathing gas flow (FlowMus). DE 10 2019 006 480 describes the estimation of these work of breathing components by means of electromyography of the respiratory muscles.

The electrical activity of the diaphragm (EAdi) is recorded by means of a modified gastric probe equipped with electrodes in order to control the pressure assistance of the ventilator proportionally to this electrical activity in processes with “Neurally Adjusted Ventilatory Assist” (NAVA), as described, e.g., in: Sinderby et al.: “Is One Fixed Level of Assist Sufficient to Mechanically Ventilate Spontaneously Breathing Patients?,” Yearbook of Intensive Care and Emergency Medicine, 2007, as well as in Sinderby et al.: “Neural Control of Mechanical Ventilation in Respiratory Failure,” Nature Medicine, 1999.

An especially proportionally assisting NAVA process with the use of a signal for the electrical activity of the diaphragm, whose peculiar feature is that the electrical activity of the diaphragm, which is needed for a defined tidal volume (the so-called neuroventilatory efficiency) shall be maintained at a constant value by means of a “closed-loop” control, is known from U.S. Pat. No. 7,021,310 B1.

Variants of how, for example, the pressure parameter Pmus or the “muscle airway pressure” or respiratory muscle pressure Pmus, pₘᵤₛ(t) can be determined arise from US 2009 159 082 AA and DE 10 2007 062 214 B3, to the explanations of which reference shall also expressly made in the description of this application concerning the disclosure concerning terms such as “muscles,” “respiratory muscles,” “muscle airway pressure,” “respiratory muscle pressure,” “parameter Pmus,” “pressure parameter Pmus,” “pressure parameter or parameter Pmus, which indicates a pressure that has been caused by a muscle breathing effort of a patient:”
a) Calculation from measured values for airway pressure, volume flow Flow(t), from which the tidal volume Vol(t) is also obtained by integration, as well as from the lung mechanical parameters R (resistance) and E (elastance).
b) Determination by equating with the negative airway pressure −Pₒₖₖ(t) measured during an occlusion, wherein the lung mechanical parameters R and E are either also calculated or are predefined.
c) Determination by means of an esophageal catheter, which is equipped with pressure sensors for measuring the intrathoracic pressure Pₑₛ(t). The esophageal catheter may optionally be configured and used for measuring the abdominal pressure P_{abd}(t).
d) Determination by means of an array of surface electrodes or sensors, which, arranged on the thorax, provide electromyographically or mechanomyographically determined electrical signals, which can correspondingly also be related to the respiratory muscle pressure Pₘᵤₛ(t) by means of suitable assignment rules, tables, functions or conversion parameters, for example, the so-called “neuromechanical efficiency,” or “neuromuscular efficiency” (NME) and correspondingly also with a volume Vₘᵤₛ(t) by means of the so-called “neuroventilatory efficiency” (NVE).
e) Determination by means of a gastric probe equipped with electrodes, which yield electrical signals, which can be related to the respiratory muscle pressure Pₘᵤₛ(t) by means of suitable assignment rules, tables, functions or conversion parameters.

The variants of the determination of Pₘᵤₛ(t) and Pmus listed in a) through e) and the additional components, such as sensors, electrodes, surface electrodes, pressure sensors, flow sensors, gastric probe, esophageal catheter, which are necessary for the determination of these variables in a device, in a system or in a process, are obtained corresponding to the variant. The breathing activity signal u_{emg}(1) can be subjected to a transformation into a pressure signal p_{emg}(u_{emg}(t)) by means of a predefined transformation rule. The transformation rule can be determined by linear or nonlinear regression between u_{emg}(t) and pₘᵤₛ(t) or also with other procedures, e.g., with neuronal networks, machine learning, or simple scaling+. For example, the following linear regression equation can be used to determine the regression coefficients for the transformation rule being sought:

$${p}_{mus} ( t ) = {a}_{0} + {a}_{1} * {u}_{emg} ( t ) + {a}_{2} * {u}_{emg}^{2} ( t ) + a 3 * {u}_{emg}^{3} ( t ) + ( t )$$

with which a transformed p_{emg}(t) signal is ultimately obtained for the further use, for example, for the ventilation control and/or for the stimulation.

### SUMMARY

Objects arise based on this, and solutions and concepts (devices, systems and processes) are provided for this by the aspects of the present invention.

Based on this, one object of the present invention is to provide an improved concept (device, system and process) for a diagnostics in a therapy process.

Another object is to provide an improved concept (device, system and process) for assisting a patient during ventilation.

Another object is to provide an improved concept (device, system and process) for a breathing assist of a patient.

Another object is to provide an improved concept (device, system and process) for a stimulative ventilatory assist of a patient.

Advantageous embodiments of the present invention will be explained in more detail below partially with reference to the figures.

The following designations will be used below, and the following variables are variable over time, as is indicated in Table 1 below.

TABLE 1

| | |
|---|---|
| Vol_{ges}, | The entire volume or the entire volume flow, which flows |
| Vol′_{ges} | from the ventilator to the patient or vice versa |
| Volₘᵤₛ, | The volume or the volume flow, which the patient achieves |
| Vol′ₘᵤₛ | with the patient's respiratory muscles, usually not |
| | measurable directly |
| Volᵥₑₙₜ, | The volume or the volume flow, which the ventilator achieves |
| Vol′ᵥₑₙₜ | |
| P_{aw} | The pressure present at the ventilator |
| P_{drv} | The entire pressure acting on the lungs (driving pressure) |
| Pᵥₑₙₜ | The pressure generated by the ventilator |
| Pₘᵤₛ | The pressure generated by the patient with the patient's |
| | respiratory muscles |
| R, E | Resistance or elasticity of the respiratory system, comprising |
| | the lungs and the thorax |
| PEEP | End-expiratory pressure |
| iPEEP | Intrinsic end-expiratory pressure, which remains in the lungs |
| | after a breath |
| τ, tau | Respiratory time constant of the respiratory system, which is |
| | considered to be a linear system |
| S | Frequency (complex number) |

The present description uses the following terms and definitions:

### (Muscle) Breathing Effort:

The term is used in the generic sense. It designates the muscle effort of the patient to generate a respiratory muscle pressure in order to bring about a flow in the airway. If occlusion is performed during the breathing effort, i.e., if the airway flow is interrupted by blockage, no flow develops in the airway, even though a respiratory muscle pressure (which can be measured as “mouth pressure” (pressure in the mouth area) if the airways are open) is generated. There is an isometric contraction of the respiratory muscles in this case. A physiological work always corresponds to the breathing effort, but the physiological work cannot be measured directly. A physical work, which is, by contrast, measurable, is only performed when the contraction is not generated isometrically, i.e., when a flow is generated in the airway.

### Breathing Assistance:

The breathing assistance is a ventilatory counterpart to the muscle breathing effort. The ventilator assists the patient's breathing effort detected (by triggering) synchronously with an assistance stroke. The patient consequently sets the breathing rhythm. It may be a pressure-controlled assistance (the airway pressure is set) or—more rarely—a volume-controlled assistance (the breath volume is set). Physical work is performed by the ventilator in all cases, i.e., a part of the total work of breathing to be performed is taken from the patient.

### (Mandatory) Ventilation:

The total work of breathing is taken from the patient during mandatory ventilation. The breathing rhythm is determined by the machine. The patient is normally passive during the mandatory ventilation, so that there will be no conflict between the human/patient and the machine. The passivity of the patient is often brought about by sedatives and relaxants.

### WOB—Work of Breathing:

This is the physiological or physical work, which is performed for the breathing or/and ventilation. Even though no physical work is performed in case of isometric contraction in the sense of the kinetic equation, as it will be described below in the further course of the description, other definitions of work, e.g., the so-called pressure-time product (time integral), can be used.

### WOBtot—Total Work of Breathing:

This is the total physiological or physical work, which is performed for the breathing or/and ventilation.

### WOBvent—Machine/Ventilator-Side Work of Breathing:

This is the component of the work of breathing that is contributed by the ventilator.

### WOBmus—Patient-Side (Muscle) Work of Breathing:

This is the component of the work of breathing that is performed solely by the patient (the patient's own efforts), both with and without stimulation of the muscles.

### WOBspon—Spontaneous (Patient-Side) Work of Breathing:

This is the work of breathing performed by spontaneous intrinsic breathing of the patient. The muscles are not stimulated in the process.

### WOBstim—Stimulated (Patient-Side) Work of Breathing:

This is the work of breathing performed by stimulation of the respiratory muscles of the patient.

### Pdrv—Driving Pressure:

Pdrv is the sum of the pressures that are generated by the ventilator and by the patient.

### Pvent—Ventilation Pressure:

Pvent is the pressure that is generated by the ventilator.

### Pmus—Muscle Pressure:

Pmus is the pressure that is generated by the muscles of the patient alone, both with and without stimulation of the muscles.

### Pspon—Spontaneous Muscle Pressure:

Pspon is the component of the muscle pressure that is generated spontaneously, i.e., without stimulation, by the patient.

### Pstim—Stimulated Muscle Pressure:

Pstim is the component of the muscle pressure that is generated by the patient solely based on the stimulation of the muscles.

### WOBbase and PmusBase—Basic Breathing Load:

The basic breathing load can be equated with the work of breathing or with the driving pressure, which is necessary to overcome the resistive, elastic and optionally other resistances and to reach a sufficient volume (e.g., the minute volume set by the clinical staff) in case of a healthy breathing pattern. The breathing pattern is preferably assumed to be an energy-optimized pattern. The work of breathing or the driving pressure can be generated on the patient side or/and on the machine side. The latter would happen in case of a (mandatory) ventilation.

### Breathing Load:

The breathing load can be determined by detecting the work of breathing or the driving pressure, which work of breathing or driving pressure is actually generated. The breathing load is normally higher than the basic breathing load, since the breathing rhythm of the patient is not energy-optimized, e.g., the patient seeks to have a higher volume, e.g., based on shortness of breath than is needed or there is an asynchronism between the patient and the ventilator. The ventilator assumes a part of the breathing load in case of breathing assistance.

### Activation Signal:

This is a signal that detects the neuronal activation of the muscle (caused either by stimulation or by spontaneous breathing effort), e.g., the (s)EMG (surface electromyogram), EIM (electrical impedance myogram), MMG (mechanomyogram). As an alternative, signals that are detected by means of novel optical or acoustic (e.g., ultrasound) technology are also considered. The enveloping curve of the EMG will be used below as an activation signal (designated by “EMG” for simplicity's sake), without excluding other signals. It shall not be ruled out hereby that there are different activation signals of different muscle groups (e.g., diaphragm and intercostal muscles), which yield an activation of their own each. The diaphragm activation signal is in the foreground in the context of the stimulation of the diaphragm (for example, by magnetic stimulation of the phrenic nerve).

### Activatability:

This is the ability electrically to stimulate (activate) the muscles, for example, by electrical or magnetic stimulation. The activation is preferably elicited by a so-called magnetic twitch stimulation, by a transient stimulation pulse with high intensity, which leads to a maximum contraction of the stimulated muscle [E11]. Other stimulation patterns may be used as well. The activation can be detected by means of an activation signal, preferably with EMG.

### Efficiency

The efficiency is a pneumatic target variable (pressure or volume), which is reached by a muscle activation. The “neuromechanical efficiency” (or “neuromuscular efficiency”) NME relates the muscle pressure generated [E18, E21], the “neuroventilatory efficiency” NVE the volume generated to the EMG [E22]. The determination of the efficiency often requires maneuvers, e.g., occlusions or changes in the breathing assistance. As is described in [E22], the values have a diagnostic significance, e.g., in the assessment of the progression of weaning from the ventilator.

### Maximum Possible Breathing Effort

This corresponds to the work of breathing WOBmusMax, to the volume VolmusMax or to the muscle pressure PmusMax, which can be achieved by maximum effort of the respiratory muscles. PmusMax can be measured most readily in a standardized manner. Thus, the value PImax is often used in the literature as the indicator of the maximum attainable muscle pressure, i.e., the maximum pressure generated during an inhalation with the mouth closed. Since the voluntary contraction of the diaphragm leads to unreliable results, a so-called (usually magnetic) twitch stimulation, which can be carried out independently from the ability of the patient to cooperate, is often used recently to trigger the contraction. PmusMax is normally detected by means of an esophageal pressure catheter, but the mouth closing pressure, which can be measured in a more simple manner, has likewise proved to be meaningful [E23]. Triggering of the stimulation is advantageous in this case [E11].

### LI Load (Load Index):

This is an indicator that depends on the ratio of the work of breathing generated to the maximum work of breathing that can be generated, and the muscle pressure or another indicator may also be used for “work.” The load can be defined for the muscle pressure as:

$$LI = Pmus / PmusMax .$$

### LBC Load-Bearing Capacity:

This is the ability of the muscles to exert by contraction a defined force or pressure—in the case of the respiratory muscles—and thus to be able to perform work. To quantify the load-bearing capacity, the muscle pressure/work of breathing generated by the muscles is usually related to the maximum muscle pressure/work of breathing that can be generated. The load-bearing capacity can thus be determined quantitatively depending on the ratio of the basic breathing load to the maximum breathing effort that can be generated. When the basic breathing load exceeds a certain portion of the maximum breathing effort that can be generated, the load-bearing capacity is not given any longer for exclusive spontaneous breathing and ventilation is mandatory. The load-bearing capacity can be defined for the muscle pressure as:

$$LBC = 1 - PmusBase / PmusMax .$$

### Exhaustion:

This occurs after some time when the load-bearing capacity of the respiratory muscles is so low that the component of the breathing load generated by the patient exceeds a certain portion of the maximum breathing effort that can be generated, e.g., 50%.

### Degree of Exhaustion:

The degree of exhaustion is linked with the load-bearing capacity, with the breathing effort and with the duration thereof, but it cannot be calculated exclusively and directly from that. However, there are measured values, which can be calculated, for example, from the electromyogram of the muscles and be used as a surrogate for the degree of exhaustion [E19, E20].

The relationships of the relevant variables may also be described on the basis of formulas, some of which will be shown below. The work of breathing can be calculated as an integral of the corresponding pressure via the volume, e.g., for the entire work of breathing:

$$WOBtot = \int Pdrv ( t ) dV = \int Pdrv ( t ) \cdot Flow ( t ) dt .$$

As an alternative (especially for the case of isometric load), the pressure-time product can be used:

WOBtot˜=∫Pdrv(t)dt.

The driving pressure is divided, analogously to the work of breathing, into the different components:

$$Pdrv = Pvent + Pmus = Pvent + Pspon + Pstim \\ WOBtot = WOBvent + WOBmus = WOBvent + WOBspon + WOBstim .$$

The flow or the volume can likewise be divided analogously to the work of breathing into the different components:

$$Flow = FlowVent + FlowMus = FlowVent + FlowSpon + SlowStim ,$$

$$and$$

$$Vol = VolVent + VolMus = VolVent + VolSpon + VolStim . \\ Pdrv = Pvent + Pmus = R \cdot Flow + E \cdot Vol + const ,$$

wherein R and E=1/C are the breathing mechanical parameters resistance and elastance (the reciprocal value of compliance) of the patient, apply to the basic kinetic equation at the breathing circuit. The equation is valid under the assumption that the respiratory system of the patient can be described as a simple RC module. When the flow or the volume (as described above) are defined as the sum of the components of the ventilator and of the patient,

$$Pvent = R \cdot FlowVent + E \cdot VolVent + const$$

$$and$$

$$Pmus = R \cdot FlowMus + E \cdot VolMus + const$$

are obtained for Pvent and Pmus.

The work of breathing of the ventilator and that of the patient can thus be calculated:

$$WOBvent = \int Pvent \cdot Flow dt = \int Pdrv \cdot FlowVent dt ,$$

$$WOBmus = \int Pmus \cdot Flow dt = \int Pdrv \cdot FlowMus dt .$$

That there are two possibilities can be derived from the kinetic equation (see above) and can be proved by insertion into the integrals. In a simplified hypothesis, Pmus can be assumed to be proportional to the EMG signal:

$$Pmus = NME \cdot EMG$$

or, for example, as a linear combination of the EMG signals of two muscle groups:

$$Pmus = NME 1 \cdot EMG 1 + NME 2 \cdot EMG 2 ,$$

wherein NME, NME1 and NME2 represent the neuromuscular efficiency of the respective muscle group. The kinetic equation

$$Pvent + Pmus = R \cdot Flow + E \cdot Vol + const$$

thus changes to

$$Pvent = R \cdot Flow + E \cdot Vol + const - NME EMG .$$

How NME can be determined is described, for example, in DE 10 2007 062 214B3, WO 2018 143 844 A, DE 10 2019 006 480A1, DE 10 2019 006 480A1, DE 10 202000 0014 A1 [E17, E18, E14, E15, E16]. The muscle activation is the sum of the spontaneous activity of the activity triggered by stimulation:

$$EMG = EMGspon + EMGstim ,$$

and it is assumed that the amplitude of the activity triggered by stimulation is linked multiplicatively with the activatability k and with the amplitude of the stimulation intensity Istim\hat{ }:

$${EMGstim}^{∧} = k \cdot {Istim}^{∧} .$$

The scalar relationship is applicable if indicators of the stimulation intensity and activation apply to broader time ranges, e.g., whole breaths. However, in this case the stimulation takes place typically as a sequence of weighted pulses with an interval of 20 msec to 100 msec corresponding to 10 Hz to 50 Hz (preferably 40 msec to 50 msec corresponding to 20 Hz to 25 Hz). Each individual pulse (twitch) triggers an individual activation, but the shape of the activation signal, which is similar to a breath, is obtained only after the entire pulse sequence. This means that the time course (time curve) of the triggered activity EMGstim(t) differs markedly from the time course of the stimulation intensity Istim(t). The activatability can only be represented as a simple constant (or characteristic) in case of variables averaged over time. A kernel-based estimation is possible for the time characteristic, e.g., with the simple hypothesis:

$$EMGstim ( t ) = Istim ( t ) * k ( t ) ,$$

wherein * represents the convolution symbol and k(t) the kernel of the modeling, which is to be estimated. Constant components (offsets) of the activation in the sense of a tonic muscle tension are ignored here. Istim(t) is usually a sequence of transient stimulation pulses. k(t) corresponds then to the pulse response of the activation to a stimulation pulse of the intensity Istim(t). There are many methods for estimating the kernel k(t), e.g., methods of system identification, stimulus-dependent averaging (e.g., analogously to the peristimulus time histogram) or least squares estimation methods.

In the equation

$$EMG = EMGspon + Istim ( t ) * k ( t )$$

EMGspon is assumed to be an error signal, which is minimized by adapting the kernel. Finally, the spontaneous activity EMGspon can also be determined in this manner, so that all factors of the kinetic equation

$$Pvent ( t ) = R \cdot Flow ( t ) + E \cdot Vol ( t ) + const - NME \cdot [ EMGspon ( t ) + k ( t ) * Istim ( t ) ]$$

are known. The components of the entire work of breathing and of the driving pressure can thus be determined and used for controlling the breathing and the stimulation. Instead of estimating the sample values of the kernel, a parametric estimation may be carried out as well. The kernel could thus be considered to be a system pulse response and its parameters could be identified.

### Correspondingly,

$$\begin{matrix}EMG & = EMG 1 + EMG 2 \\ & = EMG 1 spon + EMG 1 stim + EMG 2 spon + EMG 2 stim\end{matrix}$$

$$and$$

$$\begin{matrix}Pvent ( t ) & = R \cdot Flow ( t ) + E \cdot Vol ( t ) + const \\ & NME 1 \cdot [ EMG 1 spon ( t ) + k 1 {( t )}^{*} Istim 1 ( t ) ] \\ & NME 2 \cdot [ EMG 2 spon ( t ) + k 2 {( t )}^{*} Istim 2 ( t ) ]\end{matrix}$$

apply to the activation of, e.g., two muscle groups, possibly by means of stimulation.

The components of the work of breathing, which are performed by different muscle groups, can thus be determined. The specific stimulation makes, in fact, stimulation maneuvers possible, which pertain specifically to defined muscle groups and lead to activation. As a result, an estimation of the neuromechanical efficiencies and of the kernels of the stimulation pulse responses is comparatively simple. Instead of using work of breathing (WOB) or muscle pressure (Pmus) as target variables for the stimulation, it would also be possible, as an alternative, to use the component of the flow, which is caused by the muscles, FlowMus. FlowMus or its integral over time, the volume VolMus, may also be advantageous under some circumstances (US 2017 0252558A1 [E13]). It would be advantageous for this for the clinical staff to be very familiar with the terms flow and volume contrary to muscle pressure or work of breathing. The splitting of the flow or volume into patient components and machine components is a basis in an exemplary embodiment. When FlowMus is available, VolMus (as a time course/time course), but also VTmus (tidal volume provided by muscles) or MVmus (muscle minute volume) can also be determined in a very simple manner by an integral. These variables may be important for respiratory diagnostics and therapy.

Exemplary embodiments are based on the discovery that a state of the respiratory muscles of a patient can be characterized on the basis of a stimulation and subsequent measurement-based detection of a response of the respiratory muscles to the stimulation. Information on the state of the respiratory muscles, for example, in the form of one or more state parameters, can then be obtained from the response of the respiratory muscles.

Further exemplary embodiments are based on the discovery that the degree of utilization of the muscles of the patient can be derived from the load-bearing capacity of the respiratory muscles of the patient and from the spontaneous breathing activity (patient-attributed component of the work of breathing). When the load-bearing capacity of the muscles is higher than what would correspond to the patient-contributed component, the muscles of the patient can be loaded to a greater extent, for example, by stimulation. When the patient-contributed component is greater than the load-bearing capacity of the muscles, the muscles of the patient can be relieved, for example, by increasing the degree of sedation. By setting the actual load of the muscles, the state of the muscles can be correspondingly spared, or the muscles can be trained or loaded.

Further exemplary embodiments are based on the discovery that a respiratory muscle activation of a patient can be determined and can also be predefined within the framework of a ventilation. An indicator of a breathing assistance of the patient can then be determined from an actual respiratory muscle activation and from a desired respiratory muscle activation.

Further exemplary embodiments are based on the discovery that a time course (time course/temporal course) of an intrinsic activity of the patient (patient's own activity/efforts) P can be derived from an activation signal of the respiratory muscles of a patient. Based on the time course, an additional stimulation of the respiratory muscles can then take place in a chronological alignment (temporally coordinated). The activity of the respiratory muscles is in this case based on the intrinsic activity and on the stimulation carried out for this purpose in a chronological alignment. A synchronous and proportional stimulative ventilatory assist can be carried out in this manner. Exemplary embodiments create a device for determining a state of the respiratory muscles of a patient. The device comprises an interface arrangement comprising one or more interfaces, which are configured to detect patient signals.

Exemplary embodiments create a device for ventilating a patient. The device comprises one or more interfaces, which are configured for an exchange of information with a ventilation unit, with a stimulation unit and/or with a sensor unit.

Exemplary embodiments therefore create a device for a component of a ventilation system for the breathing assistance of a patient. The device comprises one or more interfaces for communication with components of the ventilation system.

Exemplary embodiments create a stimulator or a stimulation device for the stimulative ventilatory assist of a patient. The device comprises one or more interfaces, which are configured for the exchange of information with a ventilation unit and with a sensor unit.

Exemplary embodiments create devices in embodiments of ventilation systems, ventilation devices, stimulation devices (stimulators) for assisting a patient during the ventilation on the basis of at least one of the devices, systems, processes, computer programs or computer program products described within the framework of this description.

Exemplary embodiments create processes for assisting a patient during the ventilation with at least one of the devices, systems, processes, computer programs or computer program products described within the framework of this description. Exemplary embodiments may be configured by computer programs or computer program products with a program code for carrying out one of the processes described when the program code is executed on a computer, on a processor or on a programmable hardware component. Computer programs or computer program products may be parts of a medical system. Further elements, such as measuring devices, ventilation devices, monitoring devices, stimulation devices (stimulators), sensors for pressure measurement, sensors for flow rate measurement, sensors for detecting EMG signals (electromyogram), sEMG signals (surface electromyogram), EIM signals (electrical impedance myogram) or MMG signals (mechanomyogram) may be provided in the medical system. The exemplary embodiments or the devices may comprise a control unit.

Exemplary embodiments may comprise the detection of an indicator of a component contributed by the patient themself to the ventilation and the determination of an indicator of a load-bearing capacity of the patient. The exemplary embodiments may comprise an influencing of the component contributed by the patient themself to the ventilation and an assistance of the patient during the ventilation based on the indicator of the component contributed by the patient themself to the ventilation and based on the indicator of the load-bearing capacity of the patient. Exemplary embodiments may comprise an assistance by means of a pressure-controlled or a volume-controlled ventilation. The assistance may also comprise a stimulation of the respiratory muscles of the patient. Exemplary embodiments may comprise a control of the ventilation of the patient concerning a ventilation parameter predefined as a primary goal. Moreover, exemplary embodiments may comprise a control of the component contributed by the patient themself based on a component of the ventilation, which is predefined as a secondary goal. Furthermore, monitoring and control of the entire tidal volume of the patient, generated by the patient themself, of the work of breathing contributed by the patient themself, and/or of an oxygenation of the patient may be carried out. The influencing and the assistance may depend on a breathing rhythm predefined by the patient or on a breathing rhythm predefined by the influencing. The influencing and the assistance may depend in exemplary embodiments on a breathing rhythm predefined by the patient providing that the patient's spontaneous activity is present and is harmless, and the assistance depends otherwise on a breathing rhythm predefined by a stimulation, if a spontaneous activity of the patient is not present or is harmful and wherein the assistance depends on a breathing rhythm predefined by a pneumatic ventilation if a stimulating effect is absent.

Further exemplary embodiments may comprise a determination of an efficiency of the respiratory muscles of the patient, and the assistance is further based on the efficiency. A determination of an activatability of the respiratory muscles of the patient may, moreover, be carried out, and the assistance is further based on the activatability. In addition or as an alternative, determination of an exhaustion of the respiratory muscles of the patient may also be carried out, and the assistance is further based on the exhaustion. The determination of the load-bearing capacity (load capacity) may comprise a determination of a breathing mechanical basic load (basic respiratory mechanical load) and a detection of a maximum possible breathing effort (maximum possible respiratory effort). The detection of the maximum possible breathing effort may comprise the carrying out of a twitch stimulation.

The control unit may be configured for the stimulation of the respiratory muscles of the patient with a stimulation signal and for detecting an activation signal as a response to the stimulation. The control unit is configured, moreover, for determining one or more state parameters for the respiratory muscles based on the stimulation signal and on the activation signal. Information on the state of the respiratory muscles of the patient can reliably be obtained by the direct detection of the activation signal.

The control unit may further be configured to generate the stimulation signal with one or more stimulation pulses. The use of stimulation pulses can offer the advantage that desired responses of the muscles can be elicited by variations of the pulse sequences and pulse intensities.

The control unit may be configured for detecting an indicator of a component contributed by the patient themself to the ventilation and for determining an indicator of a load-bearing capacity of the patient. The control unit is further configured for influencing the component contributed by the patient themself to the ventilation and for assisting the patient during the ventilation based on the indicator of the component contributed by the patient themself to the ventilation and based on the indicator of the load-bearing capacity of the patient. Exemplary embodiments can thus make possible a ventilatory assist adapted to the load-bearing capacity and to the component contributed by the patient themself. For example, the control unit may be configured to output a signal for the pressure-controlled or volume-controlled ventilation for the assistance. A pneumatic ventilatory assist is thus possible. In addition or as an alternative, the control unit may be configured to output a signal for stimulating the respiratory muscles of the patient for the assistance. The ventilatory assist can thus also be carried out stimulatively or pneumatically and stimulatively.

The indicator of the work contributed by the patient themself may comprise at least one element of the group comprising
a muscle pressure, Pmus, absolute or relative to a total breathing pressure,
Paw, or a total driving pressure, Pdrv,
a breathing gas flow caused by the muscles of the patient, Flowmus, absolute or relative to a total breathing gas flow, Flow,
a tidal volume caused by the muscles of the patient, Volmus, absolute or relative to a total tidal volume, Vol, and
a work of breathing performed by the patient themself, WOBmus, absolute or relative to a total work of breathing, WOBmus.

The control unit may be configured for the detection of information on a time course of an activation signal of the respiratory muscles of the patient and for the stimulation of the respiratory muscles in a chronological alignment with the activation signal for the muscular ventilatory assist of the patient. As a result, an effective stimulative ventilatory assist can be performed. The control unit may, moreover, be configured to carry out a detection of information on a time course of a component contributed by the patient themself to the work of breathing for detecting the information on the time course of the activation signal, and to carry out a determination of the activation signal for the respiratory muscles of the patient based on information on the time course of the component contributed by the patient themself to the work of breathing. The stimulative assistance can accordingly depend on the component contributed by the patient themself. The control unit may be additionally configured here to determine a lower activation threshold for the stimulation and to take it into consideration. Activation of the respiratory muscles can take place in this case during the stimulation of the respiratory muscles above the activation threshold and activation can at least be reduced or omitted in case of a stimulation of the respiratory muscles below the activation threshold. A better chronological alignment can be obtained between the intrinsic activity and the stimulated muscle activity by taking the activation threshold into consideration.

The stimulation can be carried out by the control unit in some exemplary embodiments in a positive feedback with the activation signal. The stimulation in this case intensifies the intrinsic activity, doing so, for example, proportionally. The control unit may also be configured in this case to determine a stimulating effect. In case of a known stimulating effect, the stimulative assistance proper can be set more finely. For example, the control unit may be configured to carry out a titration for determining the stimulating effect. As a result, a stimulating effect can be taken into consideration and checked individually and also at the correct time.

In some exemplary embodiments, the control unit may be configured to carry out the stimulation proportionally to the activation signal. For example, a relative stimulative assistance, which may also depend on the load-bearing capacity of the patient, can thus then be set. For example, information on at least one element from the group comprising
a muscle pressure, Pmus,
a spontaneous muscle pressure, Pspon,
a breathing gas flow caused by the muscles of the patient, Flowmus,
a breathing gas flow caused spontaneously by the muscles of the patient, Flowspon,
a work of breathing performed by the patient themself, WOB, and
a spontaneous work of breathing performed by the patient themself, WOBspon, can be determined as information on the time course (time curve) of the activation signal of the respiratory muscles of the patient. Exemplary embodiments may offer a plurality of possibilities for determining the activation signal. In addition or as an alternative, the control unit may be configured to simultaneously ventilate the patient pneumatically. Exemplary embodiments can thus make possible a pneumatic ventilatory assist combined with a stimulative assist. The control unit may be configured to carry out the simultaneous pneumatic ventilation as a proportional ventilation. A pneumatic assistance component may also in this case depend on the intrinsic activity of the patient. For example, the detection of the information on the time course of the activation signal of the respiratory muscles of the patient can be carried out on the basis of an electromyographic signal, of a signal from an electrical impedance myography, of a signal of a strain sensor, of a signal of an ultrasound sensor or of a mechanomyographic signal. At least some of these exemplary embodiments offer the possibility of a non-invasive detection of the activation signal.

In further exemplary embodiments, the control unit may be configured to set an intensity of the stimulation on the basis of a predefined degree. A simple and effective predefinition can thus be made possible for the intensity. For example, the predefined degree is predefined on the basis of a ratio of the stimulation intensity and activation signal or on the basis of a ratio of the stimulation intensity and the estimated breathing effort, Pmus. The degree can thus correspond to a simple ratio or to a simple relationship. The degree may also define a ratio of a stimulated breathing activity to a spontaneous breathing activity of the patient. It can thus be specified that the spontaneous breathing activity of the patient is assisted by half, by a factor of two, etc.

The control unit may be configured in some exemplary embodiments to determine the spontaneous breathing activity of the patient on the basis of the information on the time course of the activation signal of the respiratory muscles of the patient. This leads to an effective possibility for determining the spontaneous breathing activity. The control unit may further be configured to determine a stimulation signal on the basis of the ratio of the stimulated breathing activity to the spontaneous breathing activity of the patient and of an activation pulse response of the muscles of the patient. An actual muscle response of the patient can thus be taken into consideration as well. For example, the control unit may be configured to determine the stimulation signal by inverse convolution of a desired activation signal caused by the stimulation, EMGstim, with the activation pulse response. This offers a simple methodical way for determining the stimulation signal. The spontaneous breathing activity can be determined, for example, as a respiratory muscle pressure, Pspon, which is generated spontaneously by the patient, and the stimulated breathing activity can be determined as a respiratory muscle pressure, Pstim, generated by stimulation. In addition or as an alternative, the control unit may be configured to determine the spontaneous breathing activity as a breathing gas flow, Flowspon, generated spontaneously by the patient, and the stimulated breathing activity as a breathing gas flow, Flowstim, generated by stimulation. It is also possible that the control unit is configured to determine the spontaneous breathing activity as a work of breathing generated spontaneously by the patient, WOBspon, or as a time derivative thereof, dWOBspon/dt, and the stimulated breathing activity as a work of breathing generated by stimulation, WOBstim, or as a time derivative thereof, dWOBstim/dt. Exemplary embodiments may use different variants to determine the spontaneous breathing activity.

The control unit may be configured in some exemplary embodiments to adjust the predefined degree on a time plane, which is greater than or equal to a breathing cycle of the patient. An adaptation of the degree of assistance is thus also possible in exemplary embodiments. The control unit may be configured to control the stimulation for reaching a target value, which comprises a patient-side component of a driving pressure ΔPmus, a patient-side component of a volume, ΔVolmus, or a patient-side component of a work of breathing, ΔWOBmus. Adaptation of the ventilatory assist which is adequate for the patient can thus take place continuously.

Exemplary embodiments also create a ventilation system with a stimulation device being described here.

Another exemplary embodiment is formed by a process for the stimulative ventilatory assist of a patient. The process comprises a detection of information on a time course of an activation signal of the respiratory muscles of the patient and a stimulation of the respiratory muscles in a chronological alignment (in temporal alignment) with the activation signal for the muscle ventilatory assist of the patient.

In further exemplary embodiments, the detection of the information on the time course of the activation signal may comprise a detection of information on a time course of a component contributed by the patient themself to the work of breathing and a determination of the activation signal for the respiratory muscles of the patient based on information on the time course of the component contributed by the patient themself to the work of breathing. The process may further comprise a determination and taking into consideration of a lower activation threshold for the stimulation, wherein activation of the respiratory muscles takes place in case of a stimulation of the respiratory muscles above the activation threshold and activation is at least reduced or is absent in case of a stimulation of the respiratory muscles below the activation threshold. The stimulation may take place in a positive feedback with the activation signal. Further, a determination of a stimulating effect can be carried out. For example, the process may also comprise a titration for determining the stimulating effect. The stimulation may take place proportionally to the activation signal.

In some exemplary embodiments, the information on the time course of the activation signal of the respiratory muscles of the patient may comprise information on at least one element from the group comprising a muscle pressure, Pmus, a spontaneous muscle pressure, Pspon, a breathing gas flow caused by the muscles of the patient, Flowmus, a breathing gas flow caused spontaneously by the muscles of the patient, Flowspon, a work of breathing performed by the patient themself, WOB, and a spontaneous work of breathing performed by the patient themself, WOBspon.

The process may comprise a simultaneous pneumatic ventilation of the patient. The simultaneous pneumatic ventilation may preferably comprise a proportional ventilation. The detection of the information on the time course of the activation signal of the respiratory muscles of the patient may be carried out, for example, on the basis of an electromyographic signal, of a signal from an electrical impedance myography, of a signal of a strain sensor, of a signal of an ultrasound sensor or of a mechanomyographic signal. The process may comprise, moreover, a setting of an intensity of the stimulation based on a predefined degree. The predefined degree may comprise a ratio of stimulation intensity and activation signal or of a stimulation intensity and an estimated breathing effort, Pmus. The degree can set, for example, a ratio between a stimulated breathing activity and a spontaneous breathing activity of the patient. Determination of the spontaneous breathing activity of the patient can be carried out in at least some exemplary embodiments on the basis of the information on the time course of the activation signal of the respiratory muscles of the patient.

In addition or as an alternative, the process may comprise a determination of a stimulation signal on the basis of the ratio between the stimulated breathing activity and the spontaneous breathing activity of the patient and an activation pulse response of the muscles of the patient. Determination of the stimulation signal may be carried out, e.g., by an inverse convolution of a desired activation signal caused by stimulation, EMGstim, with the activation pulse response. As an alternative, processing may also be carried out in the frequency range where the convolution presents itself as a multiplication. A block-by-block transformation, which may lead to delays, may be necessary for this. The spontaneous breathing activity may have a respiratory muscle pressure generated spontaneously by the patient, Pspon, and the stimulated breathing activity may have a respiratory muscle pressure generated by stimulation, Pstim. In addition or as an alternative, the spontaneous breathing activity may comprise a breathing gas flow generated by the patient spontaneously, Flowspon, and the stimulated breathing activity may comprise a breathing gas flow generated by stimulation, Flowstim. The spontaneous breathing activity may comprise a work of breathing generated spontaneously by the patient, WOBspon, or a time derivative thereof, dWOBspon/dt, and the stimulated breathing activity may comprise a work of breathing generated by stimulation, WOBstim, or a time derivative thereof, dWOBstim/dt.

In other exemplary embodiments, an adjustment of the predefined degree may take place on a time plane (temporal plane), which is greater than or equal to a breathing cycle of the patient. The process may further contain a control of the stimulation to reach a target value, which contains a patient-side component of a driving pressure, ΔPmus, a patient-side component of a volume, ΔVolmus, or a patient-side component of a work of breathing, ΔWOBmus. The control unit may be configured to determine a first piece of information on a desired respiratory muscle activation of a patient and to determine a second piece of information on an actual respiratory muscle activation of the patient. The control unit is further configured to determine an indicator of a breathing assistance of the patient based on the first information and based on the second information. Exemplary embodiments can make possible an effective breathing assistance of a patient. For example, the device may comprise a device for airway flow measurement and for airway pressure measurement at the patient. The control unit may be configured to determine the first information and the second information on the basis of an airway flow measurement and of an airway pressure measurement. Pneumatic variables can thus be used in exemplary embodiments for determining a muscle activation. The device may additionally also comprise a device for pneumatic ventilatory assistance and the indicator of the ventilatory assistance may comprise an indicator of the pneumatic ventilatory assistance. A pneumatic ventilatory assistance can thus take place in exemplary embodiments. Moreover, the device may further comprise in at least some exemplary embodiments a device for sedating the patient on the basis of the indicator of the ventilatory assistance. A basis for an automated or partially automated sedation can thus be created in exemplary embodiments. For example, measurement information on an airway flow measurement or an airway pressure measurement at the patient can be obtained via the one or more interfaces and the control unit may be configured to determine the indicator of the ventilatory assistance based on the measurement information. It may thus be possible to obtain the measurement information from other components of a ventilation system as well.

In other exemplary embodiments, the device may further comprise a device for the sensor-based detection of a signal, which depends on the actual respiratory muscle activation. The control unit may then be configured to determine the indicator of the breathing assistance on the basis of the signal detected in a sensor-based manner. It is thus also possible to take into consideration sensor-detected and especially also non-invasively detected signals in the determination of the breathing assistance. For example, the device may be configured for the sensor-based detection in order to detect an electromyogram, a mechanomyogram or an electrical impedance myogram. At least some of these signals can offer the advantage that these can be detected non-invasively. The device for the sensor-based detection may comprise, for example, a strain sensor, an ultrasound sensor or an esophageal pressure sensor.

The device may further comprise in some exemplary embodiments a device for stimulating the respiratory muscles of the patient on the basis of the indicator of the breathing assistance. In addition or as an alternative, the breathing assistance may accordingly also be carried out stimulatively.

Some exemplary embodiments can make possible a breathing control. Thus, the control unit may further be configured to reduce a difference between the first information and the second information via the indicator of the breathing assistance by means of a control. The control unit can generate, for example, a stimulation signal for the patient as an indicator of the breathing assistance. The first information and the second information can then comprise a respective indicator of a patient-side, stimulated or total respiratory muscle activation, a patient-side, stimulated or total respiratory muscle flow or a patient-side, stimulated or total respiratory muscle pressure. A spontaneous intrinsic activity of the patient can thus be taken into consideration in exemplary embodiments.

The control unit may further be configured in some exemplary embodiments to determine and to make available information on a respiratory muscle activation elicited by spontaneous breathing activity of the patient, a respiratory muscle flow elicited by spontaneous breathing activity of the patient or a respiratory muscle pressure elicited by spontaneous breathing activity of the patient. It can thus be made possible to take further into account or to process the spontaneous activity of the patient. The control unit may also be configured to determine the indicator of the breathing assistance on the basis of the information on the spontaneous breathing activity of the patient and thus to also take it into account in a possible control. The indicator of the breathing assistance may also indicate in some exemplary embodiments an indicator of a more intense sedation of the patient when the information on the spontaneous breathing activity of the patient indicates a respiratory muscle activity by means of the desired respiratory muscle activation. The muscles and the breathing apparatus of the patient can thus be protected from overstressing. The control unit may further be configured to carry out an estimation for a stimulation pulse response of the patient based on the second information in response to the indicator of the breathing assistance. Corresponding stimulation stimuli can then be determined from the stimulation pulse response and desired activations. The control unit can determine the estimation, for example, on the basis of a stimulation maneuver. Such a maneuver can make possible a precise estimation. The control unit may be configured to repeat the estimation at regular intervals or continuously. Moreover, the control unit may be configured in some exemplary embodiments to determine and to display a respective reliability for the first information and the second information when the reliability drops below a predefined threshold. This can lead to a more effective breathing assistance, because a corresponding response can be given to unreliable information.

Exemplary embodiments also create a ventilator with an exemplary embodiment of the device being described here. The functionality described for the device can then be integrated in such a ventilator.

The control unit may be configured in further exemplary embodiments to communicate information on the indicator of the breathing assistance to a stimulator via the one or more interfaces. Exemplary embodiments thus make possible a corresponding interaction of the ventilator and the stimulator. A device that makes possible a stimulation for breathing assistance is called a stimulator.

The indicator of the breathing assistance may comprise, for example, at least one parameter from the group comprising
an amplitude,
a ramp slope,
a stimulation duration,
a start time,
an end time,
an actual respiratory muscle activation, and
a desired respiratory muscle activation.
   The indicator may thus be suitable for actuating a stimulator.

The one or more interfaces may be configured for real-time communication with a stimulator and/or with a sensor unit. Rapid response of the components involved can thus be guaranteed. For example, the one or more interfaces are configured for the sample-by-sample real-time communication with a stimulator and/or with a sensor unit. In addition or as an alternative, the one or more interfaces may be configured for communication of a time course of the indicator of the breathing assistance with a stimulator and/or with a sensor unit. A sufficient chronological synchronization can be guaranteed between the components involved in the ventilatory assist. The time course describes the shape of the signal, e.g., of the stimulation intensity for the stimulator, which triggers at the start time the form of the stimulation intensity, which form was known in advance. A sample-by-sample real-time communication is thus unnecessary in this case.

Different implementations are conceivable in exemplary embodiments for the chronological coordination of the components. For example, the control unit may be configured to predefine a cycle for a stimulator via the one or more interfaces. The ventilator may also have an integrated stimulator in some exemplary embodiments. The control unit may be configured to predefine the first information and the second information for a stimulator via the one or more interfaces. In addition or as an alternative, the control unit may be configured to coordinate ventilation maneuvers with a stimulator via the one or more interfaces. Exemplary embodiments thus make possible a ventilation in a coordinated operation of a ventilator and a stimulator and possibly also a sensor unit.

Exemplary embodiments also create a stimulator with an exemplary embodiment of the device being described here. The control unit may then be configured to receive the first information and the second information from a sensor unit or from a ventilator via the one or more interfaces. A stimulator with corresponding functionality may thus also be integrated in a ventilation system.

The control unit may be configured in the stimulator to receive at least information from the group comprising an amplitude, a ramp slope, a stimulation duration, a start time, an end time, a time course of the desired respiratory muscle activation, an airway flow, an airway pressure, a respiratory muscle pressure, a respiratory muscle action and a respiratory muscle flow from a sensor unit via the one or more interfaces. A systematic interaction can thus be guaranteed with a sensor unit. The start time or the end time does not need to be an absolute time here in the sense of a time, but it is defined as an event, which triggers or ends, for example, a stimulation.

The control unit may be configured to receive a cycle from a sensor unit and/or from a ventilator via the one or more interfaces. Sufficient chronological synchronization of the components involved can thus be achieved. The stimulator can accordingly depend on a received cycle. The control unit may be configured to predefine a cycle for a sensor unit and/or for a ventilator via the one or more interfaces. The stimulator may also provide or predefine a cycle for other components in some exemplary embodiments. The control unit may also be configured here to communicate in real time via the one or more interfaces. A sufficient chronological synchronization of the components in the system can thus be achieved. The control unit may correspondingly be configured to coordinate ventilation maneuvers with a ventilator via the one or more interfaces. A ventilation maneuver can be correspondingly coordinated as a result.

Exemplary embodiments also create a sensor unit with an exemplary embodiment of the device being described here. The control unit may then be configured to receive information on at least one pneumatic signal from a ventilator via the one or more interfaces and further to determine the indicator of the breathing assistance on the basis of the information on the at least one pneumatic signal. A pneumatic signal can thus also be taken into consideration by the sensor unit during the determination of the indicator of the breathing assistance. The control unit may be configured, for example, to determine the second information on the basis of sensor signals. The sensor signals can then be used to determine the actual respiratory muscle activation. The control unit may be configured here as well to communicate in real time via the one or more interfaces and thus to establish a sufficient chronological synchronization between the components of the ventilation system. The control unit may be configured here as well to provide at least one piece of information from the group comprising an amplitude, a ramp slope, a stimulation duration, a start time, an end time, an actual respiratory muscle activation, an airway flow, an airway pressure, a respiratory muscle pressure, a respiratory muscle action and a respiratory muscle flow as an indicator of the breathing assistance for a ventilator or for a stimulator via the one or more interfaces. This can make possible a systemic interaction with the other components. The control unit may correspondingly be configured to obtain information on a maneuver from a ventilator or from a stimulator via the one or more interfaces. A coordination of maneuvers between the components can thus be made possible.

The control unit may further be configured in some exemplary embodiments to receive from a ventilator or from a stimulator as the first information or with the first information a piece of information on a gating, measurement times to be blanked out, a filtering, or a suppression of stimulation artifacts via the one or more interfaces. Disturbances in sensor signals can thus be effectively blanked out.

Exemplary embodiments also create a ventilation system with an exemplary embodiment of the device being described here. The ventilation system may comprise a ventilator as is being described here. The ventilation system may also comprise, in addition or as an alternative, a stimulator, as it is being described here, or a sensor unit, as it is being described here.

Exemplary embodiments also create a process for a component of a ventilation system for the breathing assistance of a patient. The process comprises the determination of a first piece of information on a desired respiratory muscle activation of the patient, the determination of a second piece of information on an actual respiratory muscle activation of the patient and the determination of an indicator of a breathing assistance of the patient based on the first information and based on the second information.

The process may further comprise in exemplary embodiments the determination of the first information and of the second information on the basis of an airway flow measurement and of an airway pressure measurement. The indicator of the breathing assistance may have an indicator of the pneumatic breathing assistance. The process may comprise sedation of the patient on the basis of the indicator of the breathing assistance. Moreover, the process may contain the obtaining of measurement information on an airway flow measurement or an airway pressure measurement at the patient as well as determination of the indicator of the breathing assistance, also based on the measurement information. Sensor-based detection of a signal, which depends on the actual respiratory muscle activation, and determination of the indicator of the breathing assistance based on the sensor-detected signal can be carried out. The sensor-detected signal may be, for example, an electromyogram, a mechanomyogram or an electrical impedance myogram. The detection of the sensor signal may be carried out, e.g., with a strain sensor, with an ultrasound sensor or with an esophageal pressure sensor. Stimulation of the respiratory muscles of the patient based on the indicator of the breathing assistance may also be carried out in the process in other exemplary embodiments. A difference between the first information and the second information can be reduced by control by means of the indicator of the breathing assistance. In addition or as an alternative, a stimulation signal can be generated for the patient as an indicator of the breathing assistance. The first information and the second information may comprise each an indicator of a patient-side, stimulated or total respiratory muscle activation, a patient-side, stimulated or total respiratory muscle flow or a patient-side, stimulated or total respiratory muscle pressure. The process may include a determination and a provision of information on a respiratory muscle activation caused by spontaneous breathing activity of the patient, a respiratory muscle flow caused by spontaneous breathing activity of the patient or a respiratory muscle pressure caused by spontaneous breathing activity of the patient. The process may comprise the determination of the indicator of the breathing assistance on the basis of the information on the spontaneous breathing activity of the patient. The indicator of the breathing assistance can also indicate an indicator of a more intensive sedation of the patient when the information on the spontaneous breathing activity of the patient indicates a respiratory muscle activity above the desired respiratory muscle activation. Moreover, an estimation of a stimulation pulse response of the patient can be estimated from the second information in response to the indicator of the breathing assistance. The estimation may be carried out, for example, on the basis of a stimulation maneuver. The estimation may be carried out at regular intervals or continuously. The process may also comprise a determination of a respective reliability of the first information and of the second information and indicate if the reliability drops below a predefined threshold.

Exemplary embodiments also create a process for a ventilator, which comprises one of the processes being described here. The process may comprise communication of information on the indicator of the breathing assistance to a stimulator. The indicator of the breathing assistance may have at least one parameter of the group comprising an amplitude, a ramp slope, a stimulation duration, a start time, an actual respiratory muscle activation, an end time and the desired respiratory muscle activation. The communication may also take place in the process in real time with a stimulator and/or with a sensor unit. The communication may take place, for example, in real time sample by sample with a stimulator and/or with a sensor unit. The communication may comprise a communication of a time course of the indicator of the breathing assistance with a stimulator and/or with a sensor unit. A cycle can be preset for a stimulator. The process may comprise predefining of the first information and of the second information for a stimulator and/or a coordination of a ventilation maneuver with a stimulator.

Exemplary embodiments also create a process for a stimulator, which comprises one of the processes being described here. The first information and the second information can be received in this case from a sensor unit or from a ventilator. At least one piece of information from the group comprising an amplitude, a ramp slope, a stimulation duration, a start time, an end time, an actual respiratory muscle activation, the desired respiratory muscle activation, an airway flow, an airway pressure, as respiratory muscle pressure, a respiratory muscle action, and a respiratory muscle flow can in this case be received from a sensor unit. Moreover, a cycle can be received from a sensor unit and/or from a ventilator. A cycle may also be predefined for a sensor unit and/or for a ventilator. A communication can take place in real time. This may be important especially in case of a “gating” (blanking out of artifacts/disturbances). By predefining the cycle, the sensor unit can receive information on when stimulation is carried out, i.e., on when artifacts/disturbances are to be expected. The process may comprise a coordination of a ventilation maneuver with a ventilator.

Exemplary embodiments also create a process for a sensor unit with a process being described here. The process may comprise the receipt of information on at least one pneumatic signal from a ventilator and the determination of the indicator of the breathing assistance, also based on the information on the at least one pneumatic signal. The second information can be determined based on sensor signals. The process may have communication in real time. Moreover, at least one piece of information from the group comprising an amplitude, a ramp slope, a stimulation duration, a start time, an end time, an actual respiratory muscle activation, an airway flow, an airway pressure, a respiratory muscle pressure, a respiratory muscle action and a respiratory muscle flow can be provided as an indicator of the breathing assistance for a ventilator or for a stimulator. Information on a maneuver can be obtained. For example, information on gating, measurement times to be blanked out, a filtering, or on a suppression of stimulation artifacts can be received from a ventilator or from a stimulator as the first information or with the first information.

Moreover, exemplary embodiments create a process for a ventilation system with a process being described here. The process may comprise a process being described here for a ventilator, a process being described here for a stimulator and/or a process being described here for a sensor unit. The control unit may be configured to detect the activation signal as a pulse response. The response to defined stimulation pulse sequences or else a defined stimulation pulse sequence for a desired response can be determined by means of the pulse response. The control unit may further be configured to determine an activatability of the respiratory muscles of the patient with the determination of the state parameter or of a plurality of state parameters. The activatability may be helpful for taking into consideration, for example, a degree of exhaustion (also “fatigue”) of the respiratory muscles. For example, the muscle fibers may not partially be recruited/activated. The degree of exhaustion considerably correlates with the insufficient contraction (force/pressure generation) as a consequence of an activation, i.e., even though the muscle fibers are activated, they perform poorly.

The control unit may be configured in some exemplary embodiments to take into consideration a lower activation threshold for the stimulation signal during the determination of the activatability, wherein an activation of the respiratory muscles is reduced or is not carried out during a stimulation of the respiratory muscles below the activation threshold. A stimulation can then be carried out above the activation threshold, for example, with a marked response of the respiratory muscles. When the activation threshold is known, the stimulation can be synchronized better, e.g., with a ventilation or with spontaneous breathing. For example, ramp-like curves are used for the stimulation in order to make the stimulation as pleasant as possible for the patient. However, if the ramp begins at 0, there may be a marked time delay until the response of the respiratory muscles proper, since it may take a while until the activation threshold is exceeded because of the finite ramp slope.

The control unit may further be configured in other exemplary embodiments to determine a respiratory muscle pressure, Pstim, which can be generated by stimulation, a tidal volume, Volstim, which can be generated by stimulation, and/or a work of breathing of the patient, which can be generated by stimulation. A stimulating effect and a stimulation intensity can thus be determined based on a desired degree of assistance for the stimulation. In addition or as an alternative, the control unit may be further configured to carry out a pneumatic diagnostic maneuver for determining a pneumatic ventilation parameter and to determine, furthermore, the one or more state parameters based on the pneumatic ventilation parameter. Taking into consideration the pneumatic ventilation parameter may contribute to a more reliable determination of the one or more state parameters. The pneumatic diagnostic maneuver may comprise, for example, an occlusion, a breath flow limitation, an omission of assistance of individual breaths or a variability in the breathing assistance of the patient. Many different diagnostic manoeuvres are generally conceivable in exemplary embodiments for the further determination of pneumatic parameters.

In some exemplary embodiments, the detection of the indicator of the work contributed by the patient themself can be carried out on the basis of an electromyographic signal, of a signal from an electrical impedance myography, of a mechanomyographic signal, of an ultrasound signal, of a signal of a strain sensor, or of a signal of an esophageal pressure sensor. At least some of these options offer the possibility of detecting the information or signals noninvasively.

The control unit may be configured in other exemplary embodiments to output a signal for the stimulation of the respiratory muscles of the patient for influencing the component contributed by the patient themself. The muscles of the patient can thus be activated and trained to a certain extent if this can be reconciled with the load-bearing capacity of the muscles. The control unit may also be configured to output a signal for influencing the administration of a drug for influencing the component contributed by the patient themself. Overload of the muscles and also of the pulmonary tissue can thus be prevented, and especially if the degree of the spontaneous activity of the patient exceeds the load-bearing capacity, damage to the muscles can thus be prevented. When the driving pressure (to which the muscle pressure makes a direct contribution) and hence also the tidal volume are too high, the lungs may be damaged by barotrauma or volutrauma and repeated opening and collapse of lung areas. A lung-protective ventilation avoids this. Exemplary embodiments can thus make possible a diaphragm-protective as well as lung-protective ventilation.

Different control mechanisms are conceivable in exemplary embodiments. The control unit may be configured, for example, to control the ventilation of the patient concerning a ventilation parameter predefined as a primary goal. In addition or as an alternative, the control unit may be configured to control the component contributed by the patient themself on the basis of a component of the ventilation that is predefined as a secondary goal. An efficient control can thus take place in exemplary embodiments. The entire tidal volume or the tidal volume generated by the patient themself can be monitored and controlled in this case by the control unit. Moreover, the control unit may be configured to control and to monitor the work of breathing generated by the patient themself. At the same time, an oxygenation of the patient can also be monitored and/or controlled by the control unit. A comprehensive monitoring and ventilation control can thus be carried out in exemplary embodiments. The control unit may be configured to perform the influencing and the assistance in conformity with the breathing rhythm predefined by the patient. A synchronous ventilation assist, which is thus more pleasant for the patient, can thus take place. It may also be advantageous in other exemplary embodiments to make the assist conform with a breathing rhythm predefined by the influencing, for example, when the patient has no or very slight intrinsic activity. The control unit may consequently be configured to make the influencing and the assistance conform with a breathing rhythm predefined by the patient if the patient's spontaneous activity is present and is not damaged, and otherwise to make the assistance conform with a breathing rhythm predefined by a stimulation if a spontaneous activity of the patient is not present or is harmful and in order to make the assistance conform with a breathing rhythm predefined by a pneumatic ventilation in the absence of a stimulating effect. The ventilatory assist can then be adapted and synchronized in exemplary embodiments on the basis of the state of the patient.

The control unit may be configured in some other exemplary embodiments to determine the efficiency of the respiratory muscles of the patient, wherein the assistance is based, furthermore, on the efficiency. The assistance can thus further be made conform with the efficiency of the respiratory muscles. The control unit may, moreover, be configured to determine an activatability of the respiratory muscles of the patient, and the assistance is further based on the activatability. Accordingly, the activatability can then also be taken into consideration during the ventilatory assist. In addition, or as an alternative, the control unit may be configured to determine an exhaustion of the respiratory muscles of the patient, wherein the assistance is further based on the exhaustion, and this can thus be taken into consideration as well. The control unit may be configured in some exemplary embodiments to determine a breathing-mechanical basic load to determine the load-bearing capacity and to determine a maximum possible breathing effort of the patient. The basic load can thus also be taken into consideration, for example, in case of a reasonable breathing effort. The control unit may be configured to determine the maximum possible breathing effort, e.g., by performing a twitch stimulation. An effective determination of the maximum breathing effort can thus be made possible.

In some other exemplary embodiments, the control unit may also be configured to determine, furthermore, an indicator of a load-bearing capacity of the respiratory muscles of the patient. The indicator of the load-bearing capacity can be used to prevent an overload of the respiratory muscles. The indicator of the load-bearing capacity may be based, e.g., on a relationship between a basic load, PmusBase, and a breathing effort that can be maximally generated by the patient, PmusMax, which can make possible an efficient determination of the indicator. The control unit may be configured to determine, furthermore, an indicator of an efficiency of the respiratory muscles of the patient. The efficiency can contribute, for example, to find during the ventilation a ratio of the pneumatic breathing assistance to the stimulative breathing assistance, wherein the pneumatic assistance requires work of breathing on the part of the patient and the stimulative assistance demands that the patient performs work. The indicator of the efficiency may comprise, for example, a ratio of a tidal volume that can be generated by stimulation to the activation signal. In addition or as an alternative, the indicator of the efficiency may also comprise, for example, a ratio of a respiratory muscle pressure that can be generated by stimulation to the activation signal. The control unit may, moreover, be configured to output information on the one or more state parameters via the one or more interfaces.

A detection of the progress of the therapy can be carried out in some exemplary embodiments in non-invasive therapy processes. For example, the efficiency of the respiratory muscles, the load-bearing capacity of the respiratory muscles, possibly a degree of exhaustion, etc., are monitored. Moreover, the breathing effort to be made by the patient can be predefined. For example, a volume/flow is determined from an EMG signal and WOB/Pmus/FlowMus/VolMus is then predefined. It is also possible to set a “clock unit” depending on the patient's situation and the ventilation situation. The detection/setting of the component of the breathing effort can also be carried out for the patient in exemplary embodiments spontaneously and in a stimulated manner, for example, by means of a ventilator. Moreover, automation can also be carried out with the goal of increasing the load-bearing capacity and finally weaning the patient. The breathing effort to be made can be predefined relative to the effort that can be maximally made. A rule-based system, which predefines, for example, the corresponding trajectories or a therapy plan, can thus be made available for the ventilation.

The above-mentioned explanations have been made mainly in reference to the device, but they also apply to the respective process steps of the process. Thus, the assistance may also comprise a pressure-controlled ventilation or a volume-controlled ventilation. The assistance may also comprise stimulation of the respiratory muscles of the patient. The detection of the indicator of the component contributed by the patient themself can be carried out on the basis of an electromyographic signal, of a signal from an electrical impedance myography, of a mechanomyographic signal, of an ultrasound signal, of a signal of a strain sensor, or of a signal of an esophageal pressure sensor. The influencing of the component contributed by the patient themself may comprise stimulation of the respiratory muscles of the patient and/or influencing of a drug administration. The process may further comprise a control of the ventilation of the patient concerning a ventilation parameter predefined as a primary goal. The process may also comprise a control of the component contributed by the patient themself based on a component contributed to the ventilation, which is predefined as a secondary goal. In addition or as an alternative, monitoring and control of the entire tidal volume and of the tidal volume contributed by the patient themself, of the work of breathing contributed by the patient themself and/or of an oxygenation of the patient can be carried out. In some exemplary embodiments, the influencing and the assistance may depend on a breathing rhythm predefined by the patient or on a breathing rhythm predefined by the influencing. The influencing and the assistance may depend on a breathing rhythm predefined by the patient if the spontaneous activity of the patient is present and is harmless, and the assistance depends otherwise on a breathing rhythm, predefined by a stimulation if a spontaneous activity of the patient is not present or is harmless and wherein the assistance depends on a breathing rhythm predefined by a pneumatic ventilation in the absence of a stimulating effect.

Moreover, determination of an efficiency, of an activatability and/or of an exhaustion of the respiratory muscles of the patient can take place in exemplary embodiments, wherein the assistance is further based on the efficiency, on the activatability and/or on the exhaustion. The determination of the load-bearing capacity may comprise the determination of a breathing mechanical basic load and the detection of a maximum possible breathing effort of the patient. The detection of the maximum possible breathing effort may include carrying out a twitch stimulation. Exemplary embodiments can make possible a combination of ventilation and muscle stimulation for the diagnostics. For example, exemplary embodiments can provide a mechanism for an automatic therapy process or therapy system, which uses a combination of respiratory muscle stimulation and ventilation. The mechanisms translates the target signal necessary for the therapy into a corresponding control signal (e.g., the desired muscle pressure into the stimulation intensity), so that both muscle stimulation and ventilation can be controlled in relation to their effect. Exemplary embodiments can support a system that makes possible an effective and largely automated therapy for patients. Patients may need a breathing assistance or mechanical ventilation, for example, because of an insufficient gas exchange and/or of a limited respiratory muscle pumping function. Exemplary embodiments can further create an automatic therapy process and therapy system, which comprises a combination of respiratory muscle stimulation and ventilation. An effective and largely automated therapy can thus be made possible for patients who require breathing assistance or mechanical ventilation because of an insufficient gas exchange and/or of a limited respiratory muscle pumping function. Unlike in case of conventional ventilation, the system shall adequately monitor and control both the activity of the respiratory muscles—preferably the inspiratory respiratory muscles, primarily the diaphragm—and the gas exchange in the lungs. Conventional ventilation is frequently associated with damage to the lungs (“ventilator induced lung injury,” VILI) and/or to the diaphragm (“ventilator induced diaphragm dysfunction,” VIDD). The driving pressure (sum of the ventilation pressure and the muscle pressure) may lead to an excessively high tidal volume and damage the lungs as a result. The respiratory muscles may become exhausted because of overload or become atrophied because of too little activity. In addition, lung injury frequently develops in the latter case because the necessary comprehensive positive-pressure ventilation damages the lung tissue more intensely than when the respiratory muscle follows suit with negative pressure during the inhalation.

An indicator of the work contributed by the patient themself, as well as an indicator of the respiratory muscle activation, preferably the muscle pressure, can be detected by means of sEMG or by means of another suitable technology in some exemplary embodiments. For example, the detection of the indicator of the component contributed by the patient themself and of the indicator of the muscle activation can be carried out on the basis of an electromyographic signal, of a signal from an electrical impedance myography, of a mechanomyographic signal, of an ultrasound signal, of a signal of a strain sensor, or of a signal of an esophageal pressure sensor. It would also be possible to detect the indicator of the flow, which is caused by the muscles, FlowMus, instead of the muscle pressure Pmus [E29].

In some exemplary embodiments, FlowMus can be given preference over Pmus. Another alternative would be the work of breathing (WOB), which can be calculated by integration from the muscle pressure Pmus or FlowMus as follows:

$$\begin{matrix}WOBmus & = \int Pmus ( t ) \cdot Flow ( t ) dt \\ & = \int P ( t ) \cdot FlowMus ( t ) dt .\end{matrix}$$

For reasons of simplicity, only “muscle pressure” will be dealt with below, and the other indicators will be explicitly included.

In exemplary embodiments, the indicator of the work performed by the patient themself may comprise at least one element from the group comprising:
a muscle pressure, Pmus, absolute or relative to a total breathing pressure, Paw, or a total driving pressure, Pdrv;
a breathing gas flow caused by the muscles of the patient, Flowmus, absolute or relative to a total breathing gas flow, Flow;
a tidal volume caused by the muscles of the patient, Volmus, absolute or relative to a total tidal volume, Vol; and
a work of breathing performed by the patient themself, WOBmus, absolute or relative to a total work of breathing, WOB.
   The relationship between Pmus and the driving pressure or the airway pressure is as follows:

$$\begin{matrix}When & Pmus = fac * Pdrv , \\ then & Pmus / fac = Pdrv = Paw + Pmus \\ or & Pmus = fac / ( 1 - fac ) * Paw .\end{matrix}$$

Moreover, a secondary therapy goal can be predefined (preferably in the sense of a corridor) relative to this indicator. For example, the control unit 14 is configured to control the ventilation of the patient concerning a ventilation parameter predefined as a primary goal. The control unit 14 may then be configured, at least in some exemplary embodiments, to control the component contributed by the patient themself based on a component of the ventilation predefined as a secondary goal, as it will be explained in more detail below. For example, respiratory muscle pressure beyond the spontaneous activity can be generated by means of magnetic or electrical (or other) stimulation of the respiratory muscles. The stimulation may be carried out directly by activation of the muscle fibers or indirectly by stimulating the supplying efferent nerves. The control unit is in this case configured to output a signal for stimulating the respiratory muscles of the patient for the assistance. The control unit may also be configured to output a signal for stimulating the respiratory muscles of the patient for influencing the component contributed by the patient themself.

In other exemplary embodiments, the muscle pressure generated by spontaneous breathing can possibly be lowered by the automatic administration of drugs (e.g., sedatives or relaxants). The control unit is in this case configured to output a signal for influencing the administration of a drug to influence the component contributed by the patient themself. It can be ensured by means of a connected ventilator that the patient has basically sufficient minute volume as the primary therapy goal and the oxygenation is guaranteed by an FiO₂ setting. The control unit is in this case configured to output a signal for the pressure-controlled or volume-controlled ventilation for the assistance. It can thus also be ensured that a pressure assist is ensured in case of a lack of load-bearing capacity of the respiratory muscle and a mechanical ventilation is carried out in the case of inadequate spontaneous activity and insufficient stimulating effect. Accordingly, the control unit may be configured to monitor and to control the entire tidal volume and the tidal volume generated by the patient themself, the work of breathing performed by the patient themself and/or an oxygenation of the patient. Unlike in the case of currently available therapy devices, at least some exemplary embodiments of the system being described here can offer at the same time the protection of the lungs as well as of the respiratory muscles, especially of the diaphragm, or have this protection in the focus. It can be expected that the use of this system will lead to a reduction of the number of patients affected by VILI or VIDD. An important physiological reason for the improvement of the therapy is that the diaphragm shall possibly always follow suit actively during the inhalation, depending on the load-bearing capacity, without lung injury developing (due to excessively high driving pressure). The negative pressure caused by the diaphragm has, as far as the damage to the tissue is concerned, a great advantage over positive pressure ventilation [E25].

To embody a system and a process, which makes it possible to adequately adjust and coordinate ventilation and stimulation, e.g., with a view to the respiratory muscle pressure to be generated, different components are preferably combined with one another: A ventilator, a stimulator (actuator unit), and a sensor unit. The sensor unit (e.g., sEMG amplifier) shall detect here the activation signal and calculate the respiratory muscle pressure by means of pneumatic information. By contrast, the stimulator (actuator unit) shall generate from the actual value and the desired value of a target variable (e.g., muscle activation, airway flow, esophageal pressure or muscle pressure) a stimulation signal and possibly identify the kernel or parameter of the system pulse response. The components are connected to one another via the corresponding interfaces.

A ventilation system comprises in exemplary embodiments, for example, a ventilator with the possibility of mechanical pressure-controlled or volume-controlled ventilation, of a triggered pressure assistance and possibly proportional assistance of the patient's effort. The ventilator can have a possibility of measuring the airway flow and the airway pressure and comprise the devices being described here.

If no sensor unit is available, the muscle pressure signal can nevertheless be calculated. However, the calculation is less accurate if it is based only on the pneumatic signals of the sensors of the ventilator. The ventilator may have the possibility of changing the degree of sedation of the patient. A deeper sedation reduces, for example, the spontaneous breathing activity. An attempt is normally made to ventilate the patient with the smallest possibility quantity of sedatives or relaxants. If the respiratory muscle pressure is too high and the lungs and the diaphragm may therefore be damaged, it can be reduced by administering a suitable quantity of sedatives or relaxants.

The ventilation system may also comprise in some exemplary embodiments an actuator unit (stimulator) for stimulation (e.g., electrical stimulation, stimulation by ultrasound or preferably magnetically) of the respiratory muscles, which may potentially also comprise the devices being described. The application of the actuator preferably takes place non-invasively on the body surface, e.g., by means of stimulating electrodes or coils. The actuator may be actuated directly by predefining a time-dependent stimulation intensity Istim(t). However, it is desirable to control the effect of the stimulation in a fine-grained manner. For example, an indicator of the activation of the muscles, the airway flow caused or preferably the muscle pressure achieved by the contraction may be used as the target variable. A sensor-based feedback of the target variable is necessary for this (e.g., the EMG, EIM or MMG enveloping curve read as an indicator of the muscle activation, the flow signal or the calculated muscle pressure Pmus). After predefining this time-dependent target variable, the actuator then stimulates such that the desired value will be reached, i.e., the measured (read) actual value agrees with the desired value as accurately as possible, i.e., the stimulation takes place in such a way that it achieves a defined effect. The stimulation intensity is adapted correspondingly (possibly continuously).

The muscle activation/flow/muscle pressure caused by spontaneous breathing activity is interpreted as an error signal. This error signal may be able to be displayed on a GUI (“graphical user interface”), because it has a therapeutic or/and diagnostic value. For example, stimulation would only be performed when the predefined total patient-side muscle pressure is higher than the current patient-side muscle pressure. There would be no stimulation otherwise, and the sedation could possibly be increased in order to reduce the error signal (the so-called muscle pressure). As an alternative, a volume signal could be used as an indicator of the activation of the respiratory muscles, e.g., the indicator of the volume or flow that is caused by the muscle activation (FlowMus or VolMus) [E29].

The actuator unit takes into consideration, for example, the signal quality of the signals, which are necessary for the stimulating effect. In case of an insufficient signal quality (e.g., of sEMG of the respiratory muscles as a precondition for the calculation of the muscle pressure), the attending clinical staff can be alerted to this fact (alarm). The muscle pressure to be actually generated by the patient could then be assumed possibly automatically by the ventilator within the framework of a pressure assistance (fallback).

The ventilation system may also comprise in exemplary embodiments a sensor unit for detecting the muscle activation signal, preferably the electromyogram of the diaphragm, by means of surface electrodes. The sensor unit shall operate simultaneously with the stimulation. It is therefore generally necessary to avoid stimulation artifacts in the detected stimulation signal or to remove them quasi in real time. For example, reliability indicators can also be determined in order to minimize artifact effects (e.g., due to movement) on the estimation. Furthermore, the sensor unit detects the quality of the detected muscle activation signal or of the calculated muscle pressure signal in order to be forearmed against errors and artifact effects (e.g., movement of the body, unwanted signals). The clinical staff can be notified in case of poor quality or the system can be switched over into a fallback mode.

Exemplary embodiments can create a therapy process, which, for example, as a non-invasive therapy process in which the work of breathing (absolute or relative) to be performed by the patient is predefined depending on the progress of the therapy (e.g., efficiency of the respiratory muscles, load-bearing capacity or degree of exhaustion). The minute volume, the oxygenation and/or other basic parameters of the ventilation are predefined as the primary goal of the therapy by the physician/clinical staff. In addition, the component or the absolute value (preferably in the sense of a corridor, i.e., e.g., a trajectory with margin) of the work of breathing to be performed by the patient shall be predefined as a secondary goal. This patient-side work of breathing is divided, in turn, into a spontaneous intrinsic component and a part that is caused by the stimulation of the respiratory muscles—preferably that of the diaphragm. Furthermore, the stimulation is preferably carried out magnetically by activation of the phrenic nerve at the neck.

The remaining component of the work that is necessary to reach the primary goal of the therapy is performed by the ventilator. If the patient is not able to reach the patient's therapy goal within the framework of a breathing assistance thus set, the intrinsic breathing component could be increased by means of stimulation of the inspiratory muscles. On the other hand, the work of breathing of the patient can become greater because of spontaneous breathing activity than the predefined component. The stimulation intensity is reduced in this case or the degree of sedation or the dosage of the sedatives or relaxants is possibly increased. The long-term goal is, for example, that the patient shall reach or at least rapidly regain the patient's load-bearing capacity as much as possible in order to perform the complete work of breathing themself. Thus, weaning is either no longer necessary at all or it can be achieved in a short time. An indicator of the efficiency, activatability, load-bearing capacity and degree of exhaustion is practically determined repeatedly and the component of the work of breathing that is to be generated by the patient (i.e., the sum with and without stimulation) is correspondingly adjusted. The control unit is then correspondingly configured to determine an efficiency of the respiratory muscles of the patient, wherein the assistance is further based on the efficiency. In addition or as an alternative, the control unit may be configured to determine an activatability of the respiratory muscles of the patient, and the assistance is further based on the activatability. As a further option, the control unit may be configured to determine the exhaustion of the respiratory muscles, the assistance being further based on the exhaustion. The rest of the work of breathing is performed by the ventilator. For example, an exhaustion can thus be avoided. When the diaphragm is not able to perform the required work of breathing (e.g., because of fatigue, neuronal disturbance, obstruction, restriction or other pathological conditions), a specifically set combination of muscle stimulation and pneumatic breathing assistance can still be helpful. When the patient is not able due to the patient's muscular function, e.g., because of fatigue, to trigger breaths spontaneously and thus to make an assistance possible, the procedure is changed over as a fallback to mechanical ventilation. The breathing rhythm and the complete work of breathing are then provided by the ventilator. At the same time, a slight stimulation, which is synchronized with the ventilation, should take place to avoid muscular atrophy. Both the start and the end of the breaths are taken into consideration by the synchronization. The control unit can thus be configured to carry out the influencing and the assistance depending on a breathing rhythm predefined by the patient. In addition or as an alternative, the control unit may be configured to carry out the assistance according to the breathing rhythm predefined by the influencing.

At least in some exemplary embodiments, the control unit may be configured to carry out the influencing and the assistance according to the breathing rhythm predefined by the patient, providing that the patient's spontaneous activity is present and is harmless. Otherwise, the assistance depends on a breathing rhythm, predefined by a stimulation, if a spontaneous activity of the patient is not present or is harmful, and the assistance depends on a breathing rhythm predefined by a pneumatic ventilation if a stimulating effect is absent. This is no longer an assistance in the proper sense of the word but a mandatory ventilation. When, judging from the activation signal (or pneumatic signals), an intrinsic breathing begins, there shall again be a changeover from mechanical ventilation to breathing assistance. If the patient were able based on the patient's muscular function to trigger breaths or even to breathe spontaneously independently, but the patient does not generate any muscle activation, e.g., because of a neuronal damage, the lack of spontaneous breathing can be compensated by stimulation. The ventilator detects these stimulated breathing efforts and can assist them if the patient cannot perform the entire work of breathing themself. The primary goal of the therapy is ensured in exemplary embodiments. The following process specifies how a sufficient ventilation and possibly oxygenation is guaranteed and how both the lungs (with a view to the volume) and also the diaphragm (with a view to the work of breathing) are protected. The volume is basically monitored in order to ensure protection of the lungs (VT<VTmax) and sufficient ventilation (MW>MVmin).
When the tidal volume VT becomes too high, the stimulation intensity must be reduced and sedation must possibly be increased.
When the tidal volume VT becomes too low and the respiration rate becomes at the same time too high, this is an indication of fatigue/exhaustion (Rapid Shallow Breathing). The patient-side work of breathing must be reduced (possibly mainly by reducing the stimulation intensity) and the assistance must be increased.
When the minute volume MV becomes too low, the stimulation intensity or the assistance must be increased depending on the load-bearing capacity of the muscles.

The work of breathing is basically monitored in order to protect the diaphragm, i.e., to avoid atrophy (WOBmus<WOBmin) and fatigue (WOBmus>WOBmax).
When the patient-side work of breathing becomes too low, there is a risk of atrophy and an increase is desirable (by reducing the assistance by the ventilator or/and by increasing the stimulation intensity).
When the patient-side work of breathing becomes too high, there is a risk of exhaustion/fatigue and a reduction is desirable (by increasing the assistance by the ventilator or by reducing the stimulation intensity).

Monitoring of oxygenation is possibly carried out by means of an SpO₂ sensor system. The PEEP (positive end-expiratory pressure) and the FiO₂ value are adjusted depending on the saturation value. This is preferably carried out automatically.

A plurality of instances are considered for use as the clock unit for the synchronization in exemplary embodiments. The following process specifies, for example, who or which component the “clock unit” is, i.e., who or which component predefines the breathing rhythm. The patient is preferably the clock unit when (sufficient) spontaneous activity is present.
The muscle activation is detected by the EMG and by the ventilating machine.
The stimulation is used depending on the degree of load-bearing capacity of the muscles in order to intensify the muscle activation synchronously with the spontaneous activity and to increase the load-bearing capacity.
Assistance by the ventilation then takes place synchronously with the muscle activation depending on the work of breathing to be performed and the load-bearing capacity.

As an alternative, the stimulation unit is the clock unit, even if spontaneous activity is present, especially if the breathing pattern is harmful for the patient.
The muscle activation is detected by the ventilating machine.
The stimulation unit carries out a so-called “pacing,” i.e., it predefines the breathing pattern. This is preferably protective for the lungs and is energy-optimized.
It is possible (and also probable) that the patient is involved in the lung-protective and energy-optimized stimulation pattern and synchronizes the patient's spontaneous activity therewith. If this does not happen within a predefined time period, the spontaneous activity pattern of the patient is taken over as the basis for the stimulation.
Stimulation is used depending on the degree of the load-bearing capacity of the muscles to intensify the muscle activation synchronously with the spontaneous activity and to increase the load-bearing capacity.
Assistance is offered by the ventilation synchronously with the muscle activation depending on the work of breathing to be performed and the load-bearing capacity.
   The stimulation unit is preferably the clock unit when no (sufficient) spontaneous activity is present. This requires availability of the stimulation over an unlimited time.
Stimulation is started in this case depending on the degree of the load-bearing capacity of the muscles in order to achieve an adequate muscle activation.
The breathing pattern predefined by the stimulation unit is preferably lung-protective and energy-optimized.
The muscle activation is detected by the EMG and the ventilating machine.
Assistance is performed by the ventilation synchronously with the muscle activation depending on the work of breathing to be performed and the load-bearing capacity.
   As an alternative or in case the stimulation is not available or the stimulating effect cannot be achieved, the ventilator assumes the role of the clock unit.
A controlled mechanical ventilation (no assistance) is carried out in this case.
Depending on the current load-bearing capacity of the muscles, stimulation takes place synchronously with the ventilation in order to achieve a desired muscle activation, especially in order to avoid atrophy and to increase/maintain load-bearing capacity.

The automatic ventilation and the stimulation take place in one exemplary embodiment, for example, in the following steps:
The clinical staff initially enters a primary therapy goal preferably via the GUI of the ventilator.
   The minute volume (MV), the PEEP and the percentage of oxygen in the breathing air (FiO₂) are preferably predefined by the physician/clinical staff for the primary therapy goal (maintenance of the removal of CO₂ and maintenance of oxygenation). As an alternative to the MV, it is also possible to set the tidal volume (VT) in connection with the respiration rate or the breath duration. The mere predefining of the inspiration pressure is not sufficient, because the administered volume depends on the lung-mechanical properties of the patient.
   The primary therapy goal must definitely be observed. When the patient is not able themself to ensure sufficient ventilation (removal of CO₂) and oxygenation, the ventilator must assume this role.
The clinical staff initially enters a secondary therapy goal preferably via the GUI of the ventilator.
   Using preferably the GUI of the ventilator, the clinical staff sets as the secondary therapy goal the quantity of work needed from the patient for the breathing (in the sense of the ventilation to be carried out for removing the CO₂) and how much work is needed from the ventilator.
   The components arising in a ventilation situation can be determined by means of the calculation of the muscle pressure Pmus or of the FlowMus and they can be modified and controlled by adapting parameters (e.g., stimulation intensity, degree of sedation or assistance pressure).
The secondary therapy goal can be adapted manually depending on the state of the patient—depending especially on the muscular load-bearing capacity and the lung-mechanical properties, i.e., the basic breathing load. These parameters as well as the (spontaneous and stimulated) breathing effort currently made by the patient are numerically or/and graphically available to the clinical staff. It can easily be deduced from this what load is reasonable for the patient within the framework of the therapy course.
Automation can be achieved in a simple manner by determining the maximum possible work of breathing repeatedly and relating the work of breathing to be performed by the patient to this. When the maximum possible work of breathing increases, the patient's load-bearing capacity increases and the patient can be expected to gradually perform more and more work of breathing. The patient comes closer and closer to the patient's long-term goal of complete weaning from the ventilator due to the training effect until the patient can breathe completely without assistance and be extubated. The procedure would be as follows:
   The component of the work of breathing to be performed by the patient is set at a percentage X (e.g., 50%) of the maximum breathing effort that the patient can make (e.g., WOBmusMax, VolMusMax or PmusMax). In other words, the system seeks to require the corresponding work of breathing from the patient.
   When the patient markedly exceeds the required work of breathing, an adapted sedation should be carried out—especially if damage to the lungs or to the respiratory muscles could be expected in case of continued spontaneous activity.
   When the patient cannot perform the required work of breathing due to the condition of the patient's muscles because the patient's load-bearing capacity is lower than (100%−X) of the maximum possible breathing effort, the pressure is correspondingly reduced.
   If the patient could perform the required work of breathing based on the patient's load-bearing capacity, but the spontaneous activity is not sufficient, a (triggered) adapted stimulation synchronized in time with the spontaneous activity is carried out to activate the muscles. The stimulation amplitude is controlled in this case such that the breathing effort reaches the percentage X (on average). As an alternative, this can take place by the especially physiological proportional stimulation, in which not only the amplitude, but also the time course are predefined.
   When the patient could perform the required work of breathing (at least partially) in view to the condition of the patient's muscles but no spontaneous activity is present (e.g., because of neuronal disturbance), stimulation is carried out with a predefined time pattern. When the patient can perform the work only partially, a corresponding pressure assistance is carried out, which is synchronized with the stimulated muscle activity. As an alternative, the ventilator could predefine a mandatory ventilation pattern with adapted amplitude (for example, within the framework of a volume-controlled ventilation). The stimulation would be synchronized with this ventilation pattern and set such that the stimulated muscle activity corresponds to the percentage X.
An alternative to the automated pursuit of the secondary goal of the therapy would be the integration into a weaning process. Instead of adapting, as was done until in this case, only the pressure assistance, it is also possible to adapt the stimulation with a similar rule-based scheme. Due to the additional availability of the sensor unit (preferably sEMG), the weaning process acquires additional indicators, which can make the way to the weaning safer and more rapid (efficiency, activatability, load-bearing capacity and possibly degree of exhaustion).
A further alternative is the pursuit of a predefined training or therapy plan for pursuing the secondary therapy goal [E32]. The progress of the therapy and the adjustment of the secondary therapy goal can be described and represented here, for example, by trajectories or trends, so that the clinical staff can put the expectation of the progress into a relationship with the current situation in order to control the further course of the therapy.
Further aspects of the automation
   The lung-mechanical properties, especially the resistance and elastance, determine the basic breathing load and are considerably responsible for how high the total necessary work of breathing is in order to reach, e.g., a defined minute volume (primary therapy goal). The lung-mechanical properties are therefore preferably estimated repeatedly (e.g., continuously) [E16, E17].
   To set the patient-side component of the work of breathing, an indicator of the load-bearing capacity must be known. This load-bearing capacity is determined, for example, depending on the quotient of the basic breathing load to the maximum possible breathing effort.
   The maximum possible breathing effort can be determined once or preferably repeatedly (possibly at regular intervals) by twitch stimulation and simultaneous detection of the mouth closing pressure or a Pmus estimation. Because of its invasiveness or the abrupt and uncomfortable effect, it is desirable to perform this maneuver rather rarely. The control unit can accordingly be configured to determine a breathing-mechanical basic load to determine the load-bearing capacity and to determine a maximum possible breathing effort of the patient. The control unit may further be configured to determine the maximum possible breathing effort by carrying out a twitch stimulation. The performance of a twitch stimulation may be used, for example, to be able to determine the maximum possible breathing effort independently from the patient compliance.
   Furthermore, an indicator of the current load of the patient is determined, for example, depending on the quotient of the breathing effort generated to the maximum possible breathing effort.
   It is ensured for the automatic ventilation that especially patients with low load-bearing capacity are loaded only to the extent that the muscles will not be fatigued. This is possible by the maximum work of breathing to be performed being limited to a defined percentage of the maximum possible work of breathing (e.g., 50%). The respiratory assistance is to be adapted correspondingly.

Both muscle stimulation and ventilation can be controlled in exemplary embodiments in relation to their effect for an automatic therapy process, which comprises the combination of respiratory muscle stimulation and ventilation. A target signal, which is to be predefined, can be translated for this into a corresponding control signal (e.g., a desired muscle pressure into a stimulation intensity). Such an action control is known for the ventilation [E17], but none has been known for the combination with the stimulation so far. Something similar to an action control is described for the stimulation in [E13], but it is focused exclusively on “diaphragm work” and it does not use any characterization of the control system (efficiency indicators). The proportional pressure or the proportional flow or the volume can be preferred to an indicator of the work of breathing. For reasons of familiarity, work of breathing will be used below, and the other indicators will be explicitly included and are even to be preferred. The automatic therapy process can take place in the following steps in exemplary embodiments:
The clinical staff or an automat predefines in the first step the primary therapy goal, primarily the minute volume, the oxygenation and/or other basic parameters of the ventilation.
The clinical staff or an automat predefines in the second step the secondary therapy goal, i.e., the components of the work of breathing that are to be performed by the patient and by the ventilator. As an alternative, the total work of breathing may also be predefined and additionally the work of breathing of the patient or of the ventilator. Depending on the predefined work that the ventilator must perform, the ventilator is preferably set automatically.
The deviation from the work of breathing required from the patient is determined in the third step and a corresponding response is given:
   If this deviation is positive (the patient contributes too much), actions can be taken to lower the respiratory drive. For example, sedation or relaxation can be increased with drugs, the CO₂ partial pressure can be reduced (e.g., by an extracorporeal process), the oxygen partial pressure SpO₂ can be increased by increasing the FiO₂ concentration.
   If this deviation is negative (the patient performs too little but could perform more based on the condition of the patient's muscles), the respiratory muscles are stimulated with the goal of reaching the predefined work of breathing as accurately as possible.
   If the deviation is negative (the patient's performance is too low but the patient's performance cannot be higher due to the muscles), the pressure assistance is performed appropriately by the ventilator in order to ensure a sufficient ventilation and to avoid exhaustion. It will hopefully happen in the course of the progress of the therapy later that the assistance will not be needed any longer and the patient will be able to fully contribute the required work of breathing with the patient's muscles.

Exemplary embodiments also provide a process for determining a state of the respiratory muscles of a patient. The process comprises a stimulation of the respiratory muscles of the patient with a stimulation signal and a detection of an activation signal as a response to the stimulation. The process further comprises a determination of one or more state parameters for the respiratory muscles based on the stimulation signal and the activation signal. As was already explained above on the basis of the device, the stimulation signal may comprise one or more stimulation pulses. The activation signal can be considered to be a pulse response. The determination of the activatability may comprise the taking into consideration of a lower activation threshold for the stimulation signal, and activation of the respiratory muscles is reduced or fails to occur in case of a stimulation of the respiratory muscles below the activation threshold. The determination of the one or more state parameters may comprise the determination of an activatability of the respiratory muscles of the patient.

In other exemplary embodiments, the process may further comprise the determination of the respiratory muscles pressure, Pstim, which can be generated by stimulation, of a tidal volume, which can be generated by stimulation, Volstim, and/or of a work of breathing of the patient, which can be generated by stimulation. The process may further comprise the performance of a pneumatic diagnostic maneuver and a determination of a pneumatic ventilation parameter and a determination of the one or more state parameters, further based on the pneumatic ventilation parameter. The pneumatic diagnostic maneuver may comprise an occlusion, a breath flow limitation, an omission of the assistance of individual breaths or a variability in the breathing assistance of the patient. The process may include, moreover, a determination of an indicator of a maximum possible breathing effort of the patient. The indicator of the maximum possible breathing effort of the patient may comprise, for example, a mouth closing pressure during maximum activation of the respiratory muscles.

In some exemplary embodiments, the process may further comprise a determination of an indicator of the load-bearing capacity of the respiratory muscles of the patient. The indicator of the load-bearing capacity can be based on a relationship between a basic load, PmusBase, and a maximum possible breathing effort, PmusMax, of the patient. The process may contain, moreover, the determination of an indicator of the efficiency of the respiratory muscles of the patient. The indicator of the efficiency may comprise, for example, a ratio of a tidal volume that can be generated by stimulation to the activation signal or a ratio of a respiratory muscle pressure that can be generated by stimulation to the activation signal. The process may further comprise the output of information on the one or more state parameters.

Another exemplary embodiment is a computer program with a program code for carrying out one of the processes being described here when the program code is executed on a computer, on a processor or on a programmable hardware component.

Other exemplary embodiments include a sensor unit and/or a stimulation device (stimulator).

Other exemplary embodiments include a ventilation device.

Some of the embodiments and combinations of embodiments described within the framework of this document will be discussed below in a consolidated and clear form along with concepts of the determination of states of the patient in the setting of breathing and/or ventilation as inventive, preferred and especially preferred embodiments.

The embodiment according to the present invention is formed by a device for determining a state of the respiratory muscles of a patient. The device according to the present invention has one or more interfaces, which are configured to detect patient signals. The device has a control unit, which is configured
to stimulate the respiratory muscles of the patient with a stimulation signal,
to detect an activation signal as a response to the stimulation, and
to determine one or more state parameters for the respiratory muscles based on the stimulation signal and on the activation signal.

The control unit may be configured in a preferred embodiment to generate the stimulation signal with one or more stimulation pulses.

In a preferred embodiment, the control unit may be configured to detect the activation signal as a pulse response.

In a preferred embodiment, the control unit may be configured to determine an activatability of the respiratory muscles of the patient with the determination of the one or more state parameters.

The control unit may be configured in a preferred embodiment to take into consideration a lower activation threshold for the stimulation signal during the determination of the activatability, wherein activation of the respiratory muscles takes place during stimulation of the respiratory muscles above the activation threshold and activation is at least reduced or fails to occur during a stimulation of the respiratory muscles below the activation threshold.

In a preferred embodiment, the control unit may be configured to determine
a respiratory muscle pressure, Pstim, which can be generated by stimulation,
a tidal volume, Volstim, which can be generated by stimulation, and/or
a work of breathing of the patient, which can be generated by stimulation.

The control unit may be configured in a preferred embodiment to carry out a pneumatic diagnostic maneuver to determine a pneumatic ventilation parameter and to determine the one or more state parameters based on the pneumatic ventilation parameter.

In a preferred embodiment, the pneumatic diagnostic maneuver may comprise an occlusion, a breath flow limitation, an omission of an assistance of individual breaths or a variability in the breathing assistance of the patient.

The control unit may be configured in a preferred embodiment to determine an indicator of a maximum possible breathing effort of the patient.

The indicator of the maximum possible breathing effort of the patient may comprise in one embodiment a mouth closing pressure at maximum activation of the respiratory muscles.

The control unit may be configured in a preferred embodiment to determine an indicator of the load-bearing capacity of the respiratory muscles of the patient and/or an indicator of the efficiency of the respiratory muscles of the patient.

The indicator of the load-bearing capacity can be based in a preferred embodiment on a relationship between a basic load, PmusBase, and a maximum possible breathing effort, PmusMax, of the patient.

In a preferred embodiment, the indicator of the efficiency may comprise a ratio of a tidal volume that can be generated by stimulation or respiratory muscle pressure to the activation signal.

In a preferred embodiment, the control unit may be configured to output information on the one or more state parameters via the one or more interfaces.

An embodiment according to the present invention is configured by a device for ventilating a patient. The device according to the present invention has one or more interfaces for the exchange of information with a ventilation unit, with a stimulation unit or with a sensor unit and is configured with a control unit. The control unit is configured
to detect an indicator of a component contributed by the patient themself to the ventilation,
to determine an indicator of a load-bearing capacity of the patient,
to influence the component contributed by the patient themself to the ventilation, and
to assist the patient during the ventilation based on the indicator of the component contributed by the patient themself to the ventilation and based on the indicator of the load-bearing capacity of the patient.

In a preferred embodiment, the control unit may be configured to output for the assistance a signal for the pressure-controlled or volume-controlled ventilation, or a signal for the stimulation of the respiratory muscles of the patient.

In a preferred embodiment, the indicator of the work performed by the patient themself may comprise at least one element from the group comprising
a muscle pressure, Pmus, absolute or relative to a total breathing pressure, Paw, or to a total driving pressure, Pdrv,
a breathing gas flow, Flowmus, caused by the muscles of the patient, absolute or relative to a total breathing gas flow, Flow,
a tidal volume, Volmus, caused by the muscles of the patient, absolute or relative to a total breath volume, Vol; and
a work of breathing performed by the patient themself, WOBmus, absolute or relative to a total work of breathing, WOB.

In a preferred embodiment, the detection of the indicator of the component contributed by the patient themself can be carried out on the basis of an electromyographic signal, of a signal from an electrical impedance myography, of a mechanomyographic signal, of an ultrasound signal, of a signal of a strain sensor, or of a signal of an esophageal pressure sensor.

The control unit may be configured in a preferred embodiment to output a signal for stimulating the respiratory muscles of the patient or a signal for influencing the administration of a drug for influencing the component contributed by the patient themself.

The control unit may be configured in a preferred embodiment to control the ventilation of the patient concerning a ventilation parameter predefined as a primary goal and wherein the control unit is configured to control the component contributed by the patient themself on the basis of a component of the ventilation, which component is predefined as a secondary goal.

The control unit may be configured in a preferred embodiment to monitor and to control the entire tidal volume and the tidal volume provided by the patient themself in order to control and to monitor the work of breathing generated by the patient themself, and/or to monitor and to control the oxygenation of the patient.

The control unit may be configured in a preferred embodiment to carry out the influencing and the assistance according to a breathing rhythm predefined by the patient or according to a predefined breathing rhythm.

The control unit may be configured in a preferred embodiment to carry out the influencing and the assistance according to a breathing rhythm predefined by the patient if the patient's spontaneous activity is present and is harmless, and to carry out the assistance otherwise according to a breathing rhythm predefined by a stimulation if a spontaneous activity of the patient is not present or is harmful and to carry out the assistance according to a breathing rhythm predefined by a pneumatic ventilation in the absence of a stimulating effect.

The control unit may be configured in a preferred embodiment to determine the efficiency of the respiratory muscles of the patient, wherein the assistance is based on the efficiency, wherein the control unit is configured to determine an activatability of the respiratory muscles of the patient, wherein the assistance is based on the activatability, and/or wherein the control unit is configured to determine an exhaustion of the respiratory muscles of the patient, wherein the assistance is based on the exhaustion.

The control unit may be configured in a preferred embodiment to determine a breathing mechanical basic load to determine the load-bearing capacity and to determine a maximum possible breathing effort of the patient.

The control unit may be configured in a preferred embodiment to determine the maximum possible breathing effort by carrying out a twitch stimulation.

An embodiment according to the present invention is formed by a device according to the present invention for a component of a ventilation system for breathing assistance of a patient, with one or more interfaces for communication with components of the ventilation system and with a control unit. The control unit is configured
to determine a first piece of information on a desired respiratory muscle activation of the patient,
to determine a second piece of information on an actual respiratory muscle activation of the patient, and
to determine a first indicator of a breathing assistance of the patient based on the first information and based on the second information.

A preferred embodiment may comprise a device for the airway flow measurement and for the airway pressure measurement. The control unit may be configured here to determine the first information and the second information on the basis of an airway flow measurement and of an airway pressure measurement.

A preferred embodiment may comprise a device for the pneumatic breathing assistance. The indicator of the breathing assistance may comprise an indicator of the pneumatic breathing assistance.

A preferred embodiment may comprise a device for sedating the patient based on the indicator of the breathing assistance.

A preferred embodiment may comprise a device for stimulating the respiratory muscles of the patient based on the indicator of the breathing assistance.

The device may be configured in a preferred embodiment to obtain measurement information on an airway flow measurement or on an airway pressure measurement at the patient via the one or more interfaces. The control unit may be configured here to determine the indicator of the breathing assistance on the basis of the measurement information.

A preferred embodiment may comprise a device for the sensor-based detection of a signal, which depends on the actual respiratory muscle activation. The control unit may in this case be configured to determine the indicator of the breathing assistance on the basis of the sensor-detected signal.

The device for the sensor-based detection may be configured in a preferred embodiment to detect an electromyogram, a mechanomyogram, or an electrical impedance myogram.

The device for the sensor-based detection may comprise in a preferred embodiment a strain sensor, an ultrasound sensor or an esophageal pressure sensor.

The control unit may be configured in a preferred embodiment to reduce a difference between the first information and the second information on the basis of the indicator of the breathing assistance via a control.

In a preferred embodiment, the first information and the second information may comprise each an indicator of a patient-side, stimulated or total respiratory muscle activation, a patient-side, stimulated or total respiratory muscle flow or a patient-side, stimulated or total respiratory muscle flow.

The control unit may be configured in a preferred embodiment to determine and to provide information on a respiratory muscle activation elicited by spontaneous breathing activity of the patient, on a respiratory muscle flow elicited by spontaneous breathing activity of the patient or on a respiratory muscle pressure elicited by spontaneous breathing activity of the patient. The control unit may be configured here to determine the indicator of the breathing assistance on the basis of the information on the spontaneous breathing activity of the patient, wherein the indicator of the breathing assistance indicates an indicator of a more intensive sedation of the patient when the information on the spontaneous breathing activity of the patient indicates a respiratory muscle activity above the desired respiratory muscle activation.

The control unit may be configured in a preferred embodiment to carry out an estimation (provide a representation) for the stimulation pulse response of the patient based on the second information in response to the indicator of the ventilatory assistance.

The control unit may be configured in a preferred embodiment to determine the estimation on the basis of a stimulation maneuver.

The control unit may be configured in a preferred embodiment to determine and to display a reliability each for the first information and for the second information when the reliability drops below a predefined threshold.

An embodiment according to the present invention is configured by a device for the stimulative ventilatory assistance of a patient. The device according to the present invention has one or more interfaces, which are configured for the exchange of information with a ventilating unit and with a sensor unit. The device has a control unit, which is configured
to detect information on a time course of an activation signal of the respiratory muscles of the patient, and
to stimulate the respiratory muscles in a chronological alignment with the activation signal for the muscular ventilatory assistance of the patient.

The control unit may be configured in a preferred embodiment to carry out a detection of information on a time course of a component contributed by the patient themself to the work of breathing to detect the information on the time course of the activation signal and to carry out a determination of the activation signal for the respiratory muscles of the patient on the basis of the information on the time course of the component contributed by the patient themself to the work of breathing.

The control unit may be configured in a preferred embodiment to determine a lower activation threshold for the stimulation and to take it into consideration, wherein activation of the respiratory muscles takes place in case of a stimulation of the respiratory muscles above the activation threshold and activation is at least reduced or is not carried out in case of stimulation of the respiratory muscles below the activation threshold.

The control unit may be configured in a preferred embodiment to carry out the stimulation in a positive feedback with the activation signal and/or proportionally to the activation signal.

The control unit may be configured in a preferred embodiment to determine a stimulating effect.

The control unit may be configured in a preferred embodiment to determine information on at least one element from the group comprising
a muscle pressure, Pmus,
a spontaneous muscle pressure, Pspon,
a breathing gas flow caused by the muscles of the patient, Flowmus,
a breathing gas flow caused spontaneously by the patient, Flowspon,
a work of breathing performed by the patient themself, WOB,
and a spontaneous work of breathing performed by the patient themself, WOBspon as information on the time course of the activation signal of the respiratory muscles of the patient.

The control unit may be configured in a preferred embodiment to ventilate the patient simultaneously pneumatically as a proportional ventilation.

The control unit may be configured in a preferred embodiment to set the intensity of the stimulation on the basis of a predefined degree.

The control unit may be configured in a preferred embodiment to determine the predefined degree by means of a ratio of the stimulation intensity to the activation signal or of the stimulation intensity to an estimated breathing effort, Pmus, wherein the degree specifies a ratio of a stimulated breathing activity to a spontaneous breathing activity of the patient.

The control unit may be configured in a preferred embodiment to determine the spontaneous breathing activity of the patient on the basis of the information on the time course of the activation signal of the respiratory muscles of the patient.

The control unit may be configured in a preferred embodiment to determine a stimulation signal on the basis of the ratio of the stimulated breathing activity to the spontaneous breathing activity of the patient and an activation pulse response of the muscles of the patient.

The control unit may be configured in a preferred embodiment to determine the stimulation signal by inverse convolution of a desired activation signal caused by stimulation, EMGstim, with the activation pulse response.

The control unit may be configured in a preferred embodiment
to determine the spontaneous breathing activity as a respiratory muscle pressure generated spontaneously by the patient, Pspon,
and to determine the stimulated breathing activity as a respiratory muscle pressure generated by stimulation, Pstim,
to determine the spontaneous breathing activity as a breathing gas flow generated spontaneously by the patient, Flowspon,
and to determine the stimulated breathing activity as a breathing gas flow generated by stimulation, Flowstim, and/or
to determine the spontaneous breathing activity as a work of breathing generated spontaneously by the patient, WOBspon,
or to determine the time derivative thereof, dWOBspon/dt,
to determine the stimulated breathing activity as a work of breathing generated by stimulation, WOBstim,
or to determine the time derivative thereof, dWOBstim/dt.

An embodiment may be formed by a ventilation system for assisting a patient during ventilation with a device according to the above-mentioned embodiments.

An embodiment may be formed by a ventilator, by a stimulator, and/or by a sensor unit with a device according to one of the above-mentioned embodiments. A device that makes possible a stimulation for breathing assistance is called a stimulator.

An embodiment according to the present invention is configured by a process for determining a state of the respiratory muscles of a patient
with stimulation of the respiratory muscles of the patient with a stimulation signal;
with detection of an activation signal as a response to the stimulation;
with determination of one or more state parameters for the respiratory muscles based on the stimulation signal and the activation signal.

An embodiment according to the present invention is configured by a process for ventilating a patient, with
detection of an indicator of a component contributed by the patient themself to the ventilation;
determination of an indicator of a load-bearing capacity of the patient;
influencing of the component contributed by the patient themself and the ventilation, and
assisting the patient during the ventilation based on the indicator of the component contributed by the patient themself to the ventilation and on the basis of the indicator of the load-bearing capacity of the patient.

An embodiment according to the present invention is configured by a process for a component of a ventilation system for the breathing assistance of a patient, with
determination of a first piece of information on a desired respiratory muscle activation of the patient;
determination of a second piece of information on an actual respiratory muscle activation of the patient; and
determination of an indicator of a breathing assistance of the patient on the basis of the first information and on the basis of the second information.

An embodiment according to the present invention is configured by a process for the stimulative ventilatory assistance of a patient with
determination of information on a time course of an activation signal of the respiratory muscles of the patient and
stimulation of the respiratory muscles in a chronological alignment with the activation signal for the muscular ventilatory assistance of the patient.

Other exemplary embodiments may be configured as processes, wherein the individual steps of the process can be carried out by means of a control unit or of a control module. The control unit or the control module may be configured as elements of the above-described embodiments for carrying out the process.

One embodiment is configured by a computer program with a program code for carrying out the process for determining a state of the respiratory muscles of a patient, wherein the program code can be executed on a computer, on a processor or on a programmable hardware component.

One embodiment is configured by a computer program with a program code for carrying out the process for ventilating a patient, wherein the program code can be executed on a computer, on a processor or on a programmable hardware component.

These and other embodiments may be configured by a computer program or by a plurality of computer programs with a program code for carrying out the process according to the present invention, wherein the program code may be executed on a computer, on a processor or on a programmable hardware component.

Some examples of devices and/or processes will be explained in more detail below with reference to the attached figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1a is a block diagram of a device for determining a state of respiratory muscles of a patient;

FIG. 1b is a block diagram of a device for determining an indicator of a component of the ventilation;

FIG. 1c is a block diagram of a device for determining an indicator of a breathing assistance of a patient;

FIG. 1d is a block diagram of a device for the stimulative ventilatory assistance of a patient;

FIG. 2a is a flow chart for carrying out a process for determining a state of respiratory muscles of a patient;

FIG. 2b is a flow chart for carrying out a process for ventilation with determination of an indicator of a component of the ventilation;

FIG. 2c is a flow chart for carrying out a process for determining an indicator of a breathing assistance of a patient;

FIG. 2d is a flow chart for carrying out a process for the stimulative ventilatory assistance of a patient;

FIG. 3 is a schematic overview for the ventilation of a patient and for detecting an electromyographic signal;

FIG. 4 is a schematic view of a control circuit;

FIG. 5 is a schematic view of a control;

FIG. 6 is a view of EMG signal curves (signal courses);

FIG. 7 is an enlarged detail signal curve from FIG. 6;

FIG. 8 is another enlarged detail signal curve from FIG. 6;

FIG. 9 is graph of an averaged stimulation pulse response;

FIG. 10 is a schematic view of a curve (course) of a muscle pressure;

FIG. 11 is a graph of the activatability with activation threshold.

### DESCRIPTION OF PREFERRED EMBODIMENTS

Various examples will be described in this case with detailed reference to the attached figures. The thicknesses of lines, layers and/or areas may be exaggerated in the figures for illustration. Further examples may cover modifications, equivalents and alternatives, which fall within the scope of the disclosure. Identical or similar reference numbers pertain in the entire description of the figures to identical or similar elements, which may be implemented in a comparison with one another identically or in a modified form, while they provide the same function or a similar function. It is apparent that if an element is referred to as being “connected” or “coupled” with another element, the elements may be connected or coupled directly or via one or more intermediate elements. If two elements A and B are combined with the use of an “or,” this shall be understood to mean that all possible combinations are disclosed, i.e., only A, only B as well A and B, unless something else is explicitly or implicitly defined. An alternative wording for the same combinations is “at least one of A and B” or “A and/or B.” The same applies, mutatis mutandis, to combinations of more than two elements.

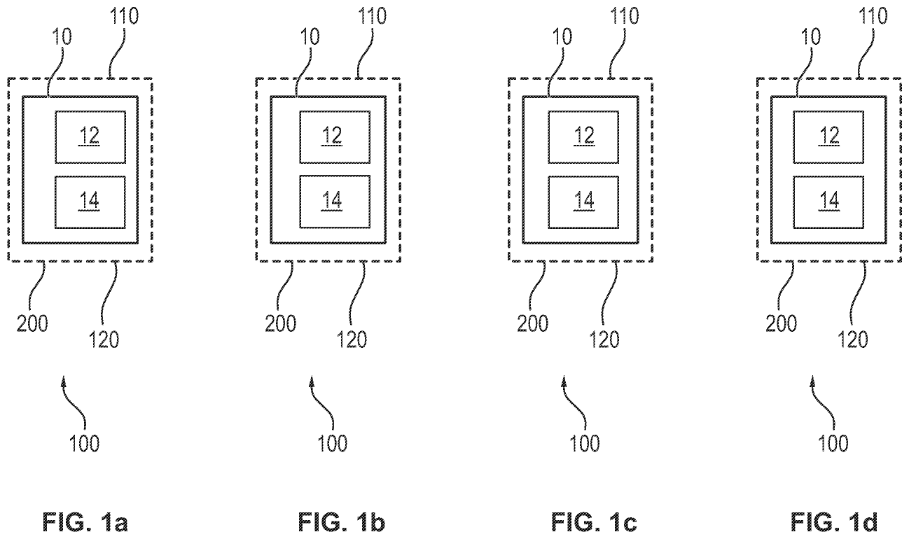

FIGS. 1a, 1b, 1c and 1d show schematic views of devices for embodiment of concepts in the context of breathing, ventilation, breathing assistance and stimulation. Identical elements in FIGS. 1a, 1b, 1c, 1d are designated by the same reference numbers in FIGS. 1a, 1b, 1c, 1d. FIG. 1a shows an exemplary embodiment of a device 100, 10. The device 10 is configured as a device for determining a state of respiratory muscles of a patient. The device 10 comprises one or more interfaces 12, which are configured for detecting patient signals. The one or more interfaces are coupled with a control unit 14. The control unit 14 is configured to stimulate the respiratory muscles of the patient with a stimulation signal and to detect an activation signal as a response to the stimulation. The control unit 14 is further configured to determine one or more state parameters for the respiratory muscles based on the stimulation signal and on the activation signal. FIG. 1a illustrates, moreover, an exemplary embodiment of a stimulation unit (stimulator) 110 or of a sensor unit 200 with a device 10. The device 10 can be integrated in a ventilation system 120 into one or more system components or it may also be implemented separately. For example, the activatability k or k(t) can be determined as a state parameter by means of an exemplary embodiment of the device 10.

FIG. 1b shows an exemplary embodiment of a device 100, 10. The device 10 is configured as a device 10 for ventilating a patient. The device 10 comprises one or more interfaces 12, which are configured for the exchange of information with a ventilation unit 120, with a stimulation unit (stimulator) 110 and/or with a sensor unit 200. The device 10 comprises, moreover, a control unit 14, which is coupled with the one or more interfaces 12. The control unit 14 is configured to detect an indicator of a component contributed by the patient themself to the ventilation. This component may be considered to be, for example, as a volume flow provided by the patient themself, Flowmus, as a muscle pressure provided by the patient themself, or as work of breathing performed by the patient themself, WOB. The control unit 14 is configured for determining an indicator of a load-bearing capacity of the patient and for influencing the component contributed by the patient themself to the ventilation. Moreover, the control unit 14 is configured to assist the patient during the ventilation on the basis of the indicator of the component contributed by the patient themself to the ventilation and based on the indicator of the load-bearing capacity of the patient.

FIG. 1c shows an exemplary embodiment of a device 100, 10. FIG. 1c shows a block diagram of an exemplary embodiment of a device 10 for a component 100 of a ventilation system 120 for breathing assistance of a patient and block diagrams of exemplary embodiments of a ventilation system 120, of a sensor unit 200 and of a stimulation unit (stimulator) 110 with such a device 10. This figure illustrates, moreover, a device 10 for a component 100 of a ventilation system 120 for breathing assistance of a patient. The device 10 comprises one or more interfaces 12 for communication with components 100 of the ventilation system 120. The device 10 comprises a control unit 14, which is coupled with the one or more interfaces 12 and which is configured to determine a first piece of information on a desired respiratory muscle activation of the patient, to determine a second piece of information on an actual respiratory muscle activation of the patient and to determine an indicator of a breathing assistance of the patient based on the first information and based on the second information.

FIG. 1d shows an exemplary embodiment of a device 100, 10. The device 10, 110 is configured as a device 110 for a stimulative ventilatory assistance of a patient. The device 10 comprises one or more interfaces 12, which are configured for the exchange of information with a ventilation unit 120 and with a sensor unit 200. The stimulation unit 110 comprises, moreover, a control unit 14, which is configured to detect information on a time course (time curve) of an activation signal of the respiratory muscles of the patient and to stimulate the respiratory muscles in a chronological alignment with the activation signal for the muscular ventilatory assistance of the patient. This figure illustrates, moreover, an exemplary embodiment of a ventilation system 120 for assisting a patient during the ventilation with a device 10. In a ventilation system 120, the device 10, 110 may be integrated in one or more system components 100 or it may also be implemented separately.

The devices 10, 100 or components 100 according to FIGS. 1a, 1b, 1c, 1d comprise one or more interfaces 12, which are coupled with the control unit 14. The one or more interfaces 12 may be configured in FIGS. 1a, 1b, 1c, 1d, for example, in the form of a machine interface or in the form of a software interface. The one or more interfaces 12 may be configured in exemplary embodiments as typical interface(s) for communication in networks or between network components or medical devices, e.g., ventilators, sensor or measuring units, stimulators, etc. For example, these may be configured in exemplary embodiments by corresponding contacts. They may also be configured in exemplary embodiments as separate hardware and may comprise a memory, which at least temporarily stores the signals to be sent or the signals received. The one or more interfaces 12 may be configured to receive electrical signals, for example, as a bus interface, as an optical interface, as an Ethernet interface, as a wireless interface, as a field bus interface, etc. It may, moreover, be configured in exemplary embodiments for wireless transmission or comprise a radio front end as well as corresponding antennas. Input and/or output devices, for example, display screen, keyboard, mouse, may also be connected via the one or more interfaces 12 in order to detect user inputs and/or outputs. The control unit 14 may comprise in exemplary embodiments one or more freely selectable controllers, microcontrollers, network processors, processor cores, such as digital signal processor cores (DSPs), programmable hardware components, etc. Exemplary embodiments are not limited to a certain type of processor core. Freely selectable processor cores or even a plurality of processor cores or microcontrollers may be provided for implementing a control unit 14. Implementations in integrated form with other devices are also conceivable, for example, in a control unit, which additionally also comprises one or more other functions. A control unit 14 may be embodied in exemplary embodiments by a processor core, a computer processor core (CPU=Central Processing Unit), a graphics processor core (GPU=Graphics Processing Unit), an application-specific integrated circuit (ASIC=Application-Specific Integrated Circuit), an integrated circuit (IC=Integrated Circuit), a one-chip system core (SOC=System on Chip), a programmable logic element or a field-programmable gate array with a microprocessor (FPGA=Field Programmable Gate Array) as a core of the component or components.

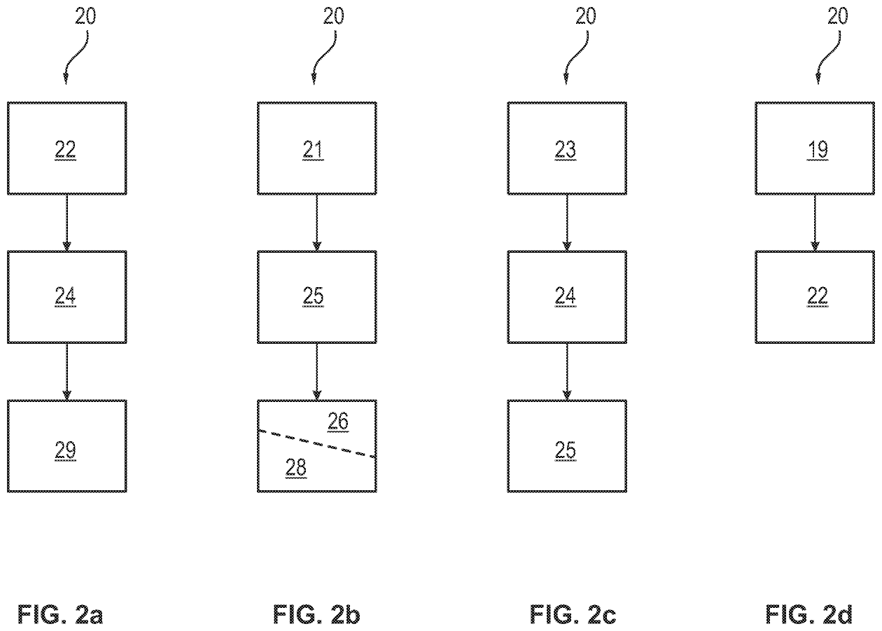

FIGS. 2a, 2b, 2c, 2d schematically show views of processes for embodying concepts in the context of breathing, ventilation, breathing assistance and stimulation. Identical elements in FIGS. 2a, 2b, 2c, 2d, 1a, 1b, 1c, 1d are designated by the same reference numbers in FIGS. 2a, 2b, 2c, 2d, 1a, 1b, 1c, 1d.

FIG. 2a shows an exemplary embodiment of a process 20 for determining a state of respiratory muscles of a patient. The process 20 comprises a stimulation 22 of the respiratory muscles of the patient with a stimulation signal, a detection 24 of an activation signal as a response to the stimulation 22 and a determination 29 of one or more state parameters for the respiratory muscles based on the stimulation signal and on the activation signal.

FIG. 2b shows an exemplary embodiment of a process 20 for ventilating a patient. The process comprises a detection 21 of an indicator of a component contributed by the patient themself to the ventilation and a determination 25 of an indicator of the load-bearing capacity of the patient. The process 20 further comprises an influencing 26 of the component contributed by the patient themself to the ventilation and an assistance 28 of the patient during the ventilation based on the indicator of the component contributed by the patient themself to the ventilation and based on the indicator of the load-bearing capacity of the patient.

FIG. 2c shows an exemplary embodiment of a process 20 for a component of a ventilation system for the breathing assistance of a patient. The process comprises a determination 23 of a first piece of information on a desired respiratory muscle activation of the patient and a determination 24 of a second piece of information on an actual respiratory muscle activation of the patient. The process 20 comprises, moreover, a determination 25 of an indicator of a breathing assistance of the patient based on the first information and based on the second information.

FIG. 2d shows an exemplary embodiment of a process 20 for the stimulative ventilatory assistance of a patient. The process 20 comprises a detection 19 of information on the time course of an activation signal of the respiratory muscles of the patient and a stimulation 22 of the respiratory muscles in a chronological alignment with the activation signal for the muscular ventilatory assistance of the patient. For example, the component contributed by the patient themself corresponds to the activation signal and contains the spontaneous and/or stimulated component.

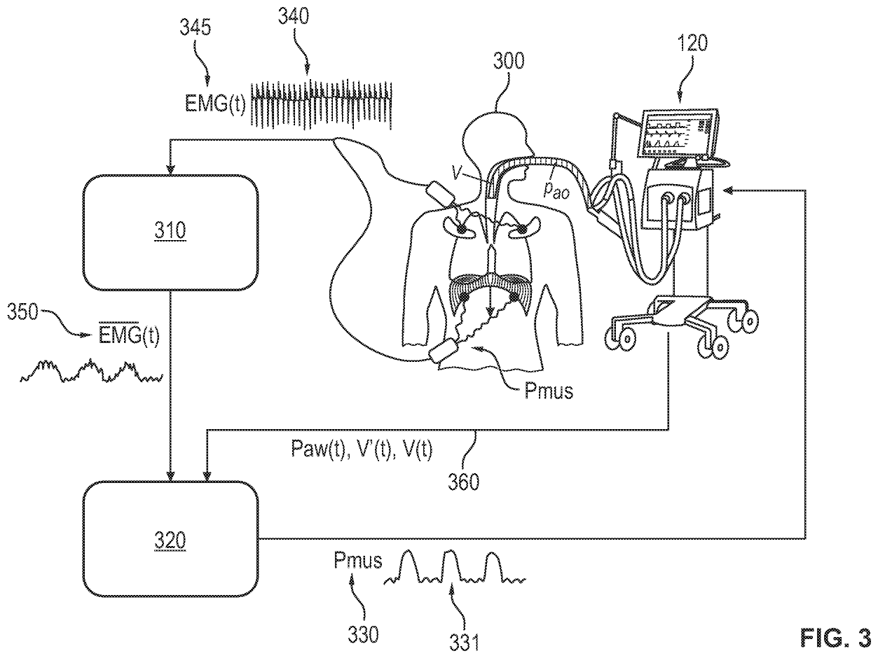

FIG. 3 shows a schematic overview figure of the ventilation of a patient and of the detection of an electromyographic signal. FIG. 3 shows a ventilation device 200, which ventilates a patient 300. An electromyographic signal 340 is detected at the respiratory muscles (diaphragm and auxiliary muscles) of the patient 300 via sensors and an original signal EMG(t) 345 is sent to a first signal processing unit 310, which determines an enveloping curve ⁻EMG(t) 350 from the original signal 345. Another signal processing unit 320 then determines a curve (course) 331 of the respiratory muscle pressure Pmus 330 of the patient 300 from the enveloping curve ⁻EMG(t) 350 and from the signals provided by the ventilating device 120 airway pressure, Paw(t), volume flow V′(t) and tidal volume V(t) 360.

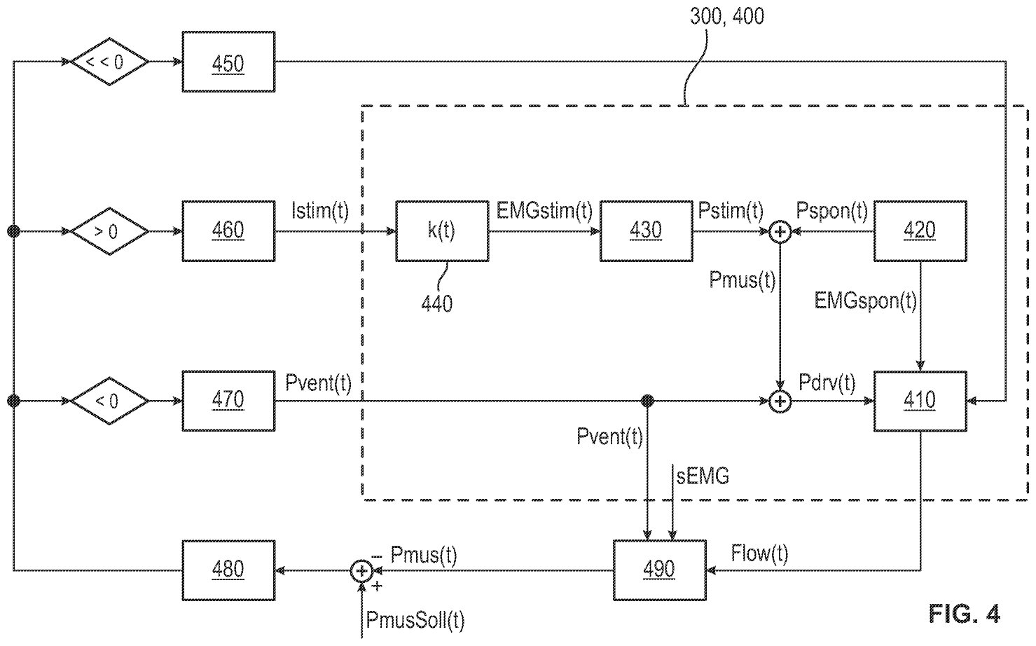

FIG. 4 shows a schematic view of a control system. The block drawn in broken lines shows the patient 300 or the patient model 400 with the respiratory system 410. A block 420 models the neuromechanical efficiency (NMEspon) of the spontaneous breathing based on an electromyographic activation signal for the spontaneous EMGspon(t), which signal is provided by the respiratory system 410. Block 420 then provides a signal Pspon(t), which characterizes the muscle pressure elicited spontaneously by the patient. The stimulated muscle pressure Pstim(t) is determined by a block 430, which models the neuromechanical efficiency (NMEstim) of the stimulation based on an activation signal EMGstim(t) generated by the stimulation. The activation signal EMGstim(t) generated by the stimulation is determined, in turn, by block 440 (k(t)) based on a stimulation intensity Istim(t) and on the activatability k(t). The entire muscle pressure Pmus(t) is then obtained from the sum of the stimulated muscle pressure Pstim(t) and of the spontaneous muscle pressure Pspon(t). Block 450 predefines a sedation, which influences the respiratory system 410. Higher sedation leads to a lower spontaneous breathing activity. Block 460 represents the stimulation and predefines the stimulation intensity Istim(t). Block 470 represents the pneumatic ventilation or breathing assistance, which ventilates the patient with the pressure Pvent(t). The pressure Pmus(t) generated by the muscles of the patient themself is then superimposed to this pressure to form the driving pressure Pdrv(t), which will finally act on the respiratory system 410. The controller 480 predefines for the components sedation 450, stimulation 460 and pneumatic breathing assistance 470 the respective manipulated variables and determines these from the control deviation at its input. The control deviation is obtained in this case from the difference of a predefined desired muscle pressure PmusSoll(t) and an estimated muscle pressure Pmus(t). This is determined by an estimator 490 based on the output pressure of the breathing assistance 470 Pvent(t), an electromyographic signal sEMG(t) detected at the patient and the measured tidal volume flow Flow(t). The device may be comprised in exemplary embodiments, for example, in the ventilator 470, in the stimulator 460 or in a sensor unit. The figure illustrates a ventilation system with these components.

A therapy system comprises a ventilator 470, a stimulator 460 and a possibility of setting the depth of sedation 450. The patient 300, 400 is assisted in the patient's breathing with the pressure Pvent. The respiratory muscles are stimulated with the intensity Istim. The flow generated, the assisting pressure and the sEMG are measured. The estimator 490 determines from these the muscle pressure Pmus. The deviation from the desired value is sent to the controller 480, which actuates the sedation 450, stimulation 460 or breathing assistance 470 depending on the polarity and the value of the result.

To make the therapy process described possible, various maneuvers can be carried out in exemplary embodiments to calculate efficiency indicators. These efficiency indicators will then make it possible to control the effect of the ventilation and stimulation. The control unit 14 may be configured to carry out a pneumatic diagnostic maneuver to determine a pneumatic ventilation parameter and to determine the one or more state parameters on the basis of the pneumatic ventilation parameter. The pneumatic diagnostic maneuver may comprise, for example, an occlusion, a breath flow limitation, an omission of an assistance of individual breaths or a variability in the breathing assistance of the patient.

The following maneuvers, as they are known, for example, from DE 10 2007 062 214 B3, WO 2018 143 844 A, DE 10 2019 006 480 A1, DE 10 2019 006 480 A1, DE 10 202000 0014 A1, may be carried out in exemplary embodiments once, repeatedly or at regular intervals.

### Maneuvers for Determining the Neuromechanical Efficiency

Neuromechanical efficiency, NME during spontaneous breathing [E18, E21, E26]: The control unit 14 may be configured to determine an indicator of an efficiency of the respiratory muscles of the patient. The indicator of the efficiency may comprise, for example, a ratio of a tidal volume that can be generated by stimulation to the activation signal. The indicator of the efficiency may also comprise, for example, a ratio of a respiratory muscle pressure that can be generated by stimulation to the activation signal.

### End-Expiratory Occlusion Maneuver:

The airway flow is blocked here at the end of the exhalation, so that the following breathing effort of the patient can be configured to be “mouth pressure” (mouth closing pressure). The maximum amplitude, the area or other parameters of the time course of the mouth pressure may be used as the indicator. This indicator related to a corresponding indicator of the activation signal (EMG) for the calculation of the NME. Repeated measurements, removal of outliers and averaging of the result may be necessary, because high variability can sometimes be expected [E21, E26]. The control unit may be configured in exemplary embodiments to determine an indicator of a maximum possible breathing effort of the patient. For example, the indicator of the maximum possible breathing effort of the patient may comprise a mouth closing pressure at maximum activation of the respiratory muscles. This (rather invasive) maneuver may be eliminated if there is sufficient variability during the spontaneous breathing and the breathing assistance. The variability may be generated as needed, e.g., by an accidental change in the pressure assistance amplitude or stimulation amplitude. It is then possible by means of estimation methods to calculate the NME [E15].

### Maneuvers for Determining the Neuroventilatory Efficiency NVE During Spontaneous Breathing [E27, E22]

The assistance is omitted here for one breath or for a plurality of breath and the tidal volume VolSpon generated (possibly averaged over several breaths) is detected and related to an indicator (mean value, area or the like) of the activation signal (EMGspon).
The quotient of the volume to the activation is determined in [E28] both during assistance and without assistance and the result related. An indicator is thus obtained for the contribution of the patient to the entire tidal volume. The idea behind this is reminiscent of the splitting of the components of the volume within the total volume, as described in [E29], wherein a true time signal is calculated in [E29] on the basis of Pmus and the omission of the assistance is not necessary.
A surrogate for the NVE can therefore also be calculated without omission of the assistance as a quotient of VolSpon to EMGspon. Since the surrogate is determined dynamically (i.e., from the time course of FlowSpon), it differs from the static NVE possibly by an offset or/and factor. The maneuver, which is not carried out so frequently, is then used to calibrate the surrogate, which yields current values continuously.
   Maneuvers for Determining the Activatability k(t):
A so-called twitch stimulation [E21], i.e., a transient stimulation pulse with high (e.g., 100%) intensity is carried out here for a short-term maximum activation of the respiratory muscles. This shall not be considered to be a restriction. Other stimulation patterns are also conceivable. Any desired stimulation pattern can ultimately be considered to be a sequence of possibly differently weighted twitches. Accordingly, the control unit 14 is then configured to generate the stimulation signal with one or more stimulation pulses. The stimulation signal may comprise, for example, one or more stimulation pulses. The control unit is in this case configured to detect the activation signal as a pulse response. The activatability of the respiratory muscles of the patient is determined with the determination of the one or more state parameters.
An activation threshold can be taken into consideration here in some exemplary embodiments. The control unit 14 is in this case configured to take into consideration a lower activation threshold for the stimulation signal during the determination of the activatability. Activation of the respiratory muscles takes place during a stimulation of the respiratory muscles above the activation threshold and an activation is at least reduced or it is completely absent during stimulation of the respiratory muscles below the activation threshold. When the activation threshold is known, the stimulation can be synchronized better, e.g., with the ventilation or with the spontaneous breathing. For example, a ramp-like curve is used for the stimulation. When such a ramp begins at 0, there may be a marked time delay, since it takes some time because of the finite ramp slope until the activation threshold is exceeded.

Exemplary embodiments can create a system and interfaces for the combination of ventilation and muscle stimulation. Exemplary embodiments can in this case provide an architecture of a system, which comprises components communicating with one another via interfaces and is configured with computing capacity. This system can make possible an effective and largely automated therapy for patients, who require breathing assistance or mechanical ventilation because of an insufficient gas exchange and/or of a limited respiratory muscle function. Unlike in conventional ventilation, the system can adequately monitor and control both the activity of the respiratory muscles—preferably the inspiratory respiratory muscles, primarily the diaphragm, and the gas exchange in the lungs. The lungs and/or the respiratory muscles are frequently damaged during conventional ventilation (“ventilator induced lung injury,” VILI as well as English “ventilator induced diaphragm dysfunction,” VIDD). The driving pressure (sum of ventilation pressure and muscle pressure) can lead, on the one hand, to high tidal volumes and thereby damage the lungs. The respiratory muscles can become exhausted because of overload or atrophied because of too little activity. Lung damage frequently develops additionally in the latter case because the necessary comprehensive positive-pressure ventilation of the pulmonary tissue is damaged to a greater extent than when the respiratory muscle follows suit during the inhalation with a negative pressure [E25].

For example, exemplary embodiments
can detect by means of sEMG (or another suitable technology) an indicator of the respiratory muscle activation, preferably the muscle pressure (the flow component that is caused by the muscles, FlowMus, could also be mentioned instead of the muscle pressure Pmus [E29]. FlowMus can be given preference over Pmus in some exemplary embodiments. Another alternative would be the work of breathing (WOB), which can be calculated from the muscle pressure Pmus or the FlowMus by integration as WOBmus=Integral Pmus(t)·Flow(t) dt=Integral P(t) FlowMus(t) dt. Only “muscle pressure” will be used below for simplicity's sake, and the other indicators will be explicitly included),
can predefine a secondary therapy goal (preferably in the sense of a corridor) relative to this indicator,
respiratory muscle pressure beyond the spontaneous activity is generated by means of magnetic or electrical (or other) stimulation of the respiratory muscles (the stimulation can take place directly by activation of the muscle fibers or indirectly by stimulation of the supplying efferent nerves),
possibly lower the muscle pressure generated by spontaneous breathing by means of the automatic administration of drugs (e.g., sedatives or relaxants),
ensure by means of a connected ventilator that
   as the primary therapy goal, the patient basically receives a sufficient minute volume and the oxygenation is guaranteed by setting the FiO₂,
   a pressure assistance is provided in case of insufficient load-bearing capacity of the respiratory muscles, and
   mechanical ventilation is carried out in case of insufficient spontaneous activity and insufficient stimulating effect.

Unlike available therapy devices, exemplary embodiments have at the same time protection of the lungs as well as of the respiratory muscles, especially of the diaphragm, in the focus. It is to be expected that their use leads to a reduction of the number of patients affected by VILI or VIDD. An important physiological reason for the improvement of the therapy is that the diaphragm shall follow suit possibly always actively—depending on the load-bearing capacity—during the inhalation, without lung injury (due to an excessively high driving pressure) developing. The negative pressure caused by the diaphragm, has, as far as the damage to the tissue is concerned, a great advantage over the positive-pressure ventilation [E25].

Different components, a ventilator, a stimulator (actuator unit) and a sensor unit, can be combined with one another in exemplary embodiments to embody a system and a process that makes it possible to adequately adjust and coordinate ventilation and stimulation, e.g., with a view to the respiratory muscle pressure to be generated. For reasons of an efficient and clear hardware/software architecture, the “intelligence,” i.e., the CPU power (computation capacity) and the algorithmics, is preferably distributed among the components. Thus, specific computation/estimation tasks shall be performed by the components that are most likely to be able for this with their available signals. The sensor unit (e.g., sEMG amplifier) shall detect the activation signal and calculate the respiratory muscle pressure by means of pneumatic information accessible via an interface. The stimulator (actuator unit) shall, by contrast, generate a stimulation signal from the actual value and the desired value of a target variable (e.g., muscle activation, airway flow or muscle pressure) and possibly identify the kernel or the parameter of the system pulse response. Consequently, the interfaces of the different components are of a special significance. The interfaces do not necessarily have to be configured for a real-time requirement (response time <50 msec), but only if this is necessary within the framework of the synchronization of activities of the communicating components. The above-described device 10 can thus be implemented in a ventilator, in a stimulator and/or in a sensor unit. Distributed implementations are also conceivable, which will then have corresponding effects on the interfaces and on the signals/information to be exchanged between the components. Thus, the device 10 may comprise, in at least some exemplary embodiments, a device for the airway flow measurement and for the airway pressure measurement at the patient, and the control unit 14 may be configured to determine the first information and the second information on the basis of an airway flow measurement and of an airway pressure measurement. The device 10 may further comprise a device for the pneumatic breathing assistance and the indicator of the breathing assistance may comprise an indicator of the pneumatic breathing assistance. For example, the ventilator has in one exemplary embodiment the possibility of a mechanical pressure-controlled ventilation, of a triggered pressure assistance and possibly proportional assistance of the patient's effort. The ventilator may have a possibility for airway flow and airway pressure measurement. The flow and pressure signals thus detected are preferably sent to the sensor unit, which is connected in terms of information technology, and which calculates a muscle pressure signal or something similar in connection with the detected activation signal. The device 10 is then configured to obtain (sensor unit) or provide (ventilator) measurement information on an airway flow measurement via the one or more interfaces 12. The control unit may be configured to determine the indicator of the breathing assistance, also based on the measurement information. As an alternative, when no separate sensor unit is available, these signals may be calculated by the ventilator itself. However, the calculation is less accurate when it is based only on the pneumatic signals of the sensors of the ventilator. The device 10 can thus be implemented in a sensor unit. The device 10 may comprise a device for the sensor-based detection of a signal, which depends on the actual respiratory muscle activation. The control unit 14 may be configured to determine the indicator of the breathing assistance on the basis of the sensor-detected signal. For example, the device for the sensor-based detection is configured to detect an electromyogram, a mechanomyogram or an electrical impedance myogram. For example, the device for the sensor-based detection comprises a strain sensor, an ultrasound sensor or an esophageal pressure sensor. Furthermore, the flow (or volume) signal and possibly the calculated muscle pressure signal or the muscle pressure signal received from the sensor unit are sent as target variables to the connected stimulator. The ventilator may have the possibility of changing the degree of sedation of the patient. A deeper sedation reduces, for example, the spontaneous breathing activity. An attempt is normally made to ventilate the patient with the smallest possible quantity of sedatives or relaxants. The device 10 further comprises in such an exemplary embodiment a device for sedating the patient based on the indicator of the breathing assistance. The indicator of the breathing assistance can indicate in exemplary embodiments an indicator of a more intensive sedation of the patient when the information on the spontaneous breathing activity of the patient indicates a respiratory muscle activity above the desired respiratory muscle activation. If the respiratory muscle pressure is too high and the lungs and the diaphragm may therefore be damaged, it can be reduced by the administration of a suitable quantity of sedatives or relaxants. The device 10 can generally be integrated into the control circuit, i.e., it may also be implemented as a controller. The control unit 14 may further be configured to reduce a difference between the first information and the second information by means of the indicator of the breathing assistance by means of a control.

Accordingly, the device 10 can be implemented in exemplary embodiments with or in a ventilator. The control unit 14 can then be configured to communicate information on the indicator of the breathing assistance to a stimulator via the one or more interfaces 12. The indicator of the breathing assistance may comprise at least one parameter of the group comprising an amplitude, a ramp slope, a stimulation duration, a start time, an end time, an actual respiratory muscle activation and the desired respiratory muscle activation. The one or more interfaces may in this case be configured for the real-time communication with a stimulator and/or with a sensor unit. Real-time communication is defined as a communication with a response time <50 msec. For example, the one or more interfaces are configured for the sample-by-sample real-time communication with a stimulator and/or with a sensor unit. The one or more interfaces may also be configured for the communication of a time course of the indicator of the breathing assistance with a stimulator and/or with a sensor unit. The control unit 14 may be configured for the chronological synchronization or coordination to predefine a cycle for a stimulator via the one or more interfaces or to receive such a cycle. Exemplary embodiments in which a stimulator is integrated in a ventilator are also conceivable. The control unit 14 may be configured to predefine the first information and the second information for a stimulator via the one or more interfaces and/or to coordinate ventilation maneuvers with a stimulator via the one or more interfaces.

The device 10 is implemented in some exemplary embodiments in or with an actuator unit (stimulator) to stimulate the respiratory muscles (e.g., electrically, by ultrasound or preferably magnetically). The control unit 14 of the device 10 can then be configured to generate a stimulation signal for the patient as an indicator of the breathing assistance and/or to comprise a device for stimulating the respiratory muscles of the patient based on the indicator of the breathing assistance. The actuator is preferably used non-invasively on the body surface, e.g., by means of stimulating electrodes or coils. The actuator may operate either independently, its stimulation can be set by predefining (non-time-critical) parameters (e.g., maximum stimulation intensity) or be actuated directly by the time-critical predefinition of a time-dependent stimulation intensity Istim(t) or of a synchronization event (e.g., stimulation start/stop). The effect of the stimulation shall preferably be controlled. For example, an indicator of the activation of the muscles, the airway flow caused or preferably the muscle pressure brought about by the contraction may be considered for use as the target variable. A sensor-based feedback of the target variable is necessary for this (e.g., the read EMG, EIM or MMG enveloping curve as the indicator of the muscle activation, the flow signal, the esophageal, gastric or differential pressure or the calculated muscle pressure Pmus). Following predefinition of this time-dependent target variable, the actuator then stimulates such that the desired value is reached, i.e., that the measured (read) actual value agrees with the desired value as accurately as possible. It is possible in this case to use a controller, which is preferably integrated in the actuator unit. The actuator unit consequently has the possibility of sending a stimulation signal to the patient as well as to read in the predefined and current target variable signal via an interface. The stimulated or the total (patient-side) muscle activation, flow or muscle pressure can in this case be selected for the target variable of the controller, and the actuator has an influence in its direct action only on the stimulated muscle activation/flow/muscle pressure. The first information and the second information (desired respiratory muscle activation, actual respiratory muscle activation) may comprise each an indicator of a patient-side, stimulated or total respiratory muscle activation, a patient-side, stimulated or total respiratory muscle flow or a patient-side, stimulated or total respiratory muscle pressure. The muscle activation/flow/muscle pressure caused by spontaneous breathing activity is then interpreted as an error signal. The control unit 14 is then configured to determine and to provide information on a respiratory muscle activation elicited by spontaneous breathing activity of the patient, a respiratory muscle flow elicited by spontaneous breathing activity or a respiratory muscle pressure elicited by spontaneous breathing activity of the patient. This error signal is preferably determined by the actuator unit and is possibly also passed on to the connected ventilator for visualization, because it has a therapeutic or/and diagnostic value. The control unit 14 is then configured to determine the indicator of the breathing assistance on the basis of the information on the spontaneous breathing activity of the patient. For example, stimulation is performed only when the predefined total patient-side muscle pressure is higher than the current patient-side muscle pressure. No stimulation would be performed otherwise. The sedation could, instead, be increased via the ventilator in order to reduce the error signal (the spontaneous muscle pressure). As an alternative, it would be possible to use a volume signal as an indicator of the activation of the respiratory muscles, e.g., the component of the volume or flow that is caused by the muscle activation (FlowMus or VolMus) [E29]. The actuator unit has to solve in all these cases an estimation task in connection with a control, because a defined effect, which is not yet known initially at the time of the delivery of the stimulation intensity, is to be achieved with the stimulation. A stimulation pulse response or a linear/nonlinear kernel k(t) in the sense of a system identification, which translates the time course of the stimulation intensity into the target variable signal, can be determined by variation of the stimulation intensity and “correlation” with the target variable signal (for example, with the detected muscle activation or with the stimulated patient-side muscle pressure signal). The control unit 14 is then configured, for example, to carry out an estimation for a stimulation pulse response of the patient based on the second information in response to the indicator of the breathing assistance. The control unit 14 is then configured to determine the estimation on the basis of a stimulation maneuver. While the pulse response can usually be parameterized (e.g., similarly to the predefinition of the P, I, D component of a PID (proportional-integral-derivative) controller or by setting the time constants), the sample values are predefined in the kernel. The variation of the stimulation intensity can be carried out by means of maneuvers, by random changes (similarly to Noisy PSV, “pressure support ventilation,” pressure-supported ventilation mode, in which the support is slightly varied randomly) or by other forms of the variability. The estimation task preferably takes place repeatedly, possibly at regular intervals or even continuously (sample value by sample value). For example, the estimation task and the control will take place only with high real-time requirement (response time <50 msec) for the embodiment of a stimulation that is proportional to the breathing effort. The control unit 14 is then configured to repeat the estimation at regular intervals or continuously. The requirements are less critical in terms of time for the adaptation of the stimulation amplitude (response time <5 sec, preferably within one breath). The actuator unit shall take into consideration the signal quality of the signals needed for the estimation during the estimation of the stimulation effect. If the signal quality (e.g., of sEMG of the respiratory muscles) is used as a precondition for the calculation of the muscle pressure, the attending clinical staff can be alerted to this (via a message or alarm). The muscle pressure actually to be generated by the patient could then possibly be taken over automatically from the ventilator within the framework of a pressure assistance (fallback). The control unit 14 is thus configured in this case to determine a respective reliability for the first information and for the second information and to indicate when the reliability drops below a predefined threshold. Corresponding to the above description, the control unit 14 may be configured to receive the first information and the second information from a sensor unit or from a ventilator via the one or more interfaces 12. The control unit 14 may further be configured in this case to receive at least one piece of information from the group comprising an amplitude, a ramp slope, a stimulation duration, a start time, an end time, a time course of the desired respiratory muscle activation, an airway flow, an airway pressure, a respiratory muscle pressure, a respiratory muscle action and a respiratory muscle flow from a sensor unit via the one or more interfaces. A cycle can be received from a sensor unit and/or from a ventilator via the one or more interfaces 12 or it can be communicated to these in at least some exemplary embodiments in real time via the one or more interfaces 12. A ventilation maneuver can also be coordinated with a ventilator in some exemplary embodiments via the one or more interfaces 12.

Exemplary embodiments also create a sensor unit with a device 10. The sensor unit is configured to detect the muscle activation signal, preferably the electromyogram of the diaphragm by means of surface electrodes. As an alternative, the EIM (electrical impedance myogram), MMG (mechanomyogram) may be considered, or even signals that are detected by means of novel optical or acoustic (e.g., ultrasound) technology. The envelope (also called enveloping curve) of the respective original signal is preferably used as an indicator of the muscle activation. For example, the control unit 14 may be configured to provide at least one piece of information from the group comprising an amplitude, a ramp slope, a stimulation duration, a start time, an end time, an actual respiratory muscle activation, an airway flow, an airway pressure, a respiratory muscle pressure, a respiratory muscle action and a respiratory muscle flow as an indicator of the breathing assistance to a ventilator or to a stimulator via the one or more interfaces 12. Following the above description, the control unit 14 may also be configured on the side of the sensor unit in order to obtain information on a maneuver from a ventilator or from a stimulator via the one or more interfaces 12. Moreover, the control unit 14 may be configured to receive information on at least one pneumatic signal from a ventilator via the one or more interfaces 12 and to determine, furthermore, the indicator of the breathing assistance based on the information on the at least one pneumatic signal. The control unit can determine, for example, the second information (actual respiratory muscle activation) on the basis of the sensor signals. The sensor unit shall operate simultaneously with the stimulation. It is therefore generally necessary to avoid stimulation artifacts in the detected stimulation signal (e.g., due to a type of “gating” or filtering) or to remove it quasi in real time. The sensor unit, just like the actuator unit, is preferably equipped with a computing unit of its own corresponding to the desired architecture to process the detected muscle activation signals, i.e., to remove, for example, artifacts, to calculate enveloping curves or to determine trigger/cycling off times. If pneumatic signals read in simultaneously are available, it is possible to calculate additional indicators, e.g., work of breathing, muscle pressure, asynchronism and lung mechanics as well as other diagnostic values in a model-based manner. The sensor unit therefore preferably has a powerful computing unit, which is necessary for the signal processing and especially for the estimation tasks, as well as a bidirectional interface [E31]. As was already described above, the control unit 14 may be configured to communicate in real time via the one or more interfaces 12. Furthermore, the sensor unit detects the quality of the detected muscle activation signal in order to be forearmed against errors and artifact effects (e.g., movement of the body, unwanted signals). The control unit 14 may be configured to receive additionally information on a gating, measurement times to be blanked out, a filtering, or a suppression of stimulation artifacts from a ventilator or from a stimulator via the one or more interfaces 12. The quality of the pneumatic signals read in can additionally be taken into consideration. This makes possible a robust estimation. The clinical staff can be notified in case of poor quality or the system can be switched over into a fallback mode.

Exemplary embodiments also create a ventilation system with a device 10. Different implementations and combinations of the components of the ventilation system are conceivable here, some of which will be explained in more detail below. The ventilation system may generally comprise in exemplary embodiments a ventilator, a stimulator and/or a sensor unit according to the present description.

At least two of the three components (ventilator, stimulator, sensor unit) together form a system in some exemplary embodiments. Three possible combinations will be described in more detail within the framework of this description: For example, a ventilator and a stimulator are combined. The stimulator may be integrated in the ventilator or (especially in the case of magnetic stimulation) it may be an external device. The GUI of the ventilator is used to enter and follow up the primary goal of the therapy (e.g., ensuring a minimal minute volume and the oxygenation). Furthermore, a secondary therapy goal is entered there (e.g., the work of breathing to be performed by the patient or the muscle pressure). The stimulator does not absolutely require a GUI.

However, if it is available, the GUI can visualize the stimulation intensity and the follow-up of the secondary therapy goal. The ventilator provides the stimulator with the amplitude (or other parameters such as ramp slope, stimulation duration, etc.), the times (events) for the start and stop of the stimulation or even the time course of the target variable, which the stimulator shall generate by adaptation of the time-dependent stimulation intensity. When the ventilator is a clock unit (clock unit for the synchronization of the components involved in the ventilation system), it may be necessary for the communication from the ventilator to the stimulator to take place in real time with a guaranteed response time of <50 msec. This is necessary, for example, when the ventilator administers strokes triggered by the spontaneous breathing of the patient or mandatory strokes and the stimulator shall be active during the inhalation phases. A stimulation proportional to the spontaneous breathing effort likewise requires real-time capability. This is not necessary (or it is necessary with a long guaranteed response time of <5 sec only) when the stimulator is a clock unit and only the stimulation amplitude (or other parameters) must be adjusted. When the stimulator is not a clock unit, it is preferably signaled to the stimulator for each breath to be assisted by stimulation in real time when the breath begins, when it ends and what the amplitude of the target variable is. Furthermore, additional parameters, which describe the form of the stimulation stroke (e.g., ramp slope), can be transmitted. The amplitude may be a scalar value or else a time series, which describes the curve of the target variable, as it would be necessary in case of proportional stimulation in real time. The stimulator is suitable in this case for responding in real time within <50 msec to the signals and to carry out the stimulation correspondingly. The airway flow (or, which is more or less the same, the volume) or the muscle pressure estimated by the ventilator, Pmus (or FlowMus, WOB) can be used as a target variable. The stimulator consequently receives simultaneously an actual value and a desired value for the target variable from the ventilator and seeks to change the actual value by the stimulation within the framework of a control, which takes place within the stimulator, such that it will be as close to the desired value as possible. If the target variable is not a scalar value but a time course, the control can detect the mean value or other statistical indicators of the actual value and control the stimulation such that the predefined desired value will be reached as closely as possible. Maneuvers, e.g., the omission of assistance strokes or mandatory strokes of the ventilator as well as a variation of the stimulation may be necessary for the identification of the control system (i.e., of the system between the stimulation intensity and the activation signal). The ventilation maneuvers are requested, for example, by the stimulator from the ventilator, which then performs the maneuvers, depending on the therapy situation. The actual value and the desired value are provided, for example, by the ventilator (e.g., flow or volume, possibly Pmus).

The stimulator and the sensor unit are combined in another exemplary embodiment. The stimulator may also be used without a ventilator. It is assumed in this case that ventilation is not obligatory and the primary therapy goal is consequently absent. The secondary therapy goal can be set via the GUI of the stimulator. The sensor unit preferably supplies the stimulator with the activation signal, either as an amplitude, as times (events) for start and end of the stimulation or even the time course, which the stimulator shall generate by adapting the time-dependent stimulation intensity, as the target variable. The sensor unit may provide the signals airway flow and airway pressure and possibly the calculated muscle pressure Pmus (or WOB, FlowMus). This would possibly require an additional sensor system. When the sensor unit is a clock unit, it is necessary for the communication of the sensor unit with the stimulator to take place in real time with a guaranteed response time of <50 msec. This is necessary, for example, when the stimulation shall be synchronized with the time course of the activation signal or with the start-stop events, i.e., in case the stimulation being proportional to the spontaneous breathing effort. Real time is not necessary (or only with a long guaranteed response time of <5 sec) when the stimulator is a clock unit and only the stimulation amplitude (or other parameters) must be adjusted. When the stimulator is not a clock unit, the start of the breath, the end of the breath and the amplitude of the target variable are signalized in real time to the stimulator preferably for each breath to be assisted by stimulation and the time course is possibly transmitted (details as in the above exemplary embodiment with the combination of ventilator and sensor unit). The stimulator consequently receives an actual value for the target variable from the sensor unit. The desired value is set by the stimulator as a consequence of the secondary therapy goal. The stimulator seeks to change within the framework of a control, which takes place within the stimulator, the actual value by the stimulation such that it will be as close to the desired value as possible. No ventilation maneuvers can be used for the control of the stimulation intensity in this configuration. Stimulation maneuvers are possible, e.g., the stimulation can take place simultaneously with or with a time shift from the spontaneous breathing activity. The analysis of the activation signal will then make it possible to determine the component of the activation that has been due to the stimulation. This is to be taken into consideration in the control, since the spontaneous component of the activation can be interpreted as an error signal. The actual value is predefined, for example, by the sensor unit and the desired value by the stimulator (activation signal).

The ventilator, the stimulator and the sensor unit are combined in another exemplary embodiment. The configuration corresponds to the above description. However, primarily the muscle pressure (Pmus) or a similar indicator (e.g., WOB, FlowMus), which is calculated from the pneumatic signals and the signals provided by the sensor unit, is used as the target variable. The calculation necessary for this is carried out preferably in the sensor unit, which receives the necessary pneumatic signals from the ventilator. The target variable can be represented in its actual value by parameters (amplitude, slope, etc.) or as a time course. The stimulator receives as above (exemplary embodiment with the combination of ventilator and stimulator) simultaneously an actual value (scalar or as a time course/time course) and a desired value for the target variable from the ventilator and seeks to change the actual value within the framework of the control, which takes place within the stimulator, by the stimulation such that it will be as close to the desired value as possible. The actual value may, as an alternative, also be supplied directly by the sensor unit.

When the ventilator or the sensor unit is a clock unit, it is necessary for the communication of the ventilator or of the sensor unit with the stimulator to take place in real time with a guaranteed response time of <50 msec. This is necessary, for example, when the ventilator administers strokes triggered by the spontaneous breathing of the patient or mandatory strokes and the stimulator shall be active during the inhalation phase. A stimulation proportional to the spontaneous breathing effort likewise requires real-time capability. This is not necessary (or is necessary only with a long guaranteed response time of <5 sec) when the stimulator is a clock unit and only the stimulation amplitude (or other parameters) must be adjusted. For further details, see the exemplary embodiment above with the combination of ventilator and stimulator. Preferably the muscle pressure Pmus (or FlowMus, WOB) estimated by the sensor unit is used as the target variable. The stimulator consequently receives simultaneously an actual value and a desired value for the target variable from the ventilator and from the sensor unit and seeks within the framework of a control, which takes place within the stimulator, to change the actual value by stimulation such that it will be as close to the desired value as possible. When the target variable is not a scalar value but a time course (time curve), the control can detect the mean value or other statistical indicators of the actual value and control the stimulation such that the predefined desired value will be reached as closely as possible. Maneuvers, e.g., the omission of assistance strokes or mandatory strokes of the ventilator as well as a variation of the stimulation, may be necessary for the identification of the control system (i.e., of the system between stimulation intensity and activation signal). The ventilation maneuvers are requested by the stimulator from the ventilator, which then performs the maneuvers, doing so depending on the therapy situation. Stimulation maneuvers are requested from the stimulator in the reverse direction by the ventilator (or by the sensor unit). The actual value is predefined by the ventilator (possibly by the sensor unit) and the desired value by the ventilator (Pmus).

Exemplary embodiments can thus make possible a therapy process. The above-described system comprising a combination of at least two devices (ventilator, stimulator, sensor unit), which combination is connected by interfaces, is suitable for the automation of a therapy process. The interfaces can—but do not always have to—meet real-time requirements. The therapy process preferably takes place non-invasively. The work of breathing to be performed by the patient (absolute or relative) is predefined depending on the progress of the therapy (e.g., efficiency of the respiratory muscles, load-bearing capacity or degree of exhaustion). The minute volume, the oxygenation and/or another basic parameter of the ventilation is predefined by the physician as the primary therapy goal. In addition, the component or the absolute value (preferably in the sense of a corridor, i.e., e.g., a trajectory with margin) of the work of breathing to be performed by the patient may additionally also be predefined as a secondary goal. This patient-side work of breathing is, in turn, divided into a spontaneous intrinsic component and a part that is caused by the stimulation of the respiratory muscles—preferably the diaphragm. Furthermore, the stimulation is preferably carried out magnetically by activation of the phrenic nerve at the neck. The remaining component of the work, which is necessary to reach the primary therapy goal, is provided by the ventilator. When the patient is in this case not able to reach the patient's therapy goal within the framework of a breathing assistance thus set, the intrinsic breathing component could be increased by means of stimulation of the respiratory muscles. On the other hand, the work of breathing of the patient can become higher based on spontaneous breathing activity than the predefined component. The intensity of stimulation is reduced in this case or possibly the degree of sedation or the rate of sedative or relaxant administration is possibly increased. The long-term goal is that the patient preserve the patient's load-bearing capacity as much as possible or at least regain it rapidly in order to perform the complete work of breathing themself. Thus, weaning is either no longer necessary or it can be achieved in a short time. An indicator of the efficiency, activatability, load-bearing capacity and the degree of exhaustion is practically determined repeatedly and the component of the work of breathing to be performed by the patient (i.e., the sum with and without stimulation) is correspondingly adapted. The rest of the work of breathing is performed by the ventilator. This can be automated, e.g., a therapy system can be used for this. For example, exhaustion can thus be avoided. When the diaphragm is not able to perform the required work of breathing (e.g., because of fatigue, neuronal disturbance, obstruction, restriction or other pathological conditions), a specifically set combination of muscle stimulation and pneumatic breathing assistance can still be helpful. When the patient is not able due to the condition of the patient's muscles, e.g., because of fatigue, to trigger breaths spontaneously and thus make an assistance possible, the procedure is switched over to mechanical ventilation as a fallback. The breathing rhythm and the complete work of breathing are then performed by the ventilator. At the same time, a mild stimulation, which is synchronized with the ventilation, should be performed to avoid muscular atrophy. The synchronization of the stimulation takes into account both the start and the end of the breaths. When, judging from the activation signal (or pneumatic signals), intrinsic breathing begins, it will again be necessary to change over from mechanical ventilation to breathing assistance. Even if the patient were able to trigger strokes or even breathe independently based on the condition of the patient's muscles, but the patient does not generate muscle activation, e.g., due to a neuronal injury, the lack of spontaneous breathing can be compensated by stimulation. The ventilator detects these stimulated breathing efforts and can assist them if the patient cannot perform the total work of breathing themself.

Exemplary embodiments provide a ventilation system and components thereof, for example, a ventilator, a stimulator and/or a sensor unit. Stimulation can then take place in exemplary embodiments with the aim of activating the muscles (estimator/controller). Corresponding indicators can in this case be determined for the muscle activation: EMG, Flow/Volume, Pmus/FlowMus, WOB, etc. Exemplary embodiments can define and provide here the corresponding interfaces as well as the information and signals to be transmitted for this.

Different combinations of components of a ventilation system may occur in exemplary embodiments, for example, combinations of ventilator and stimulator, wherein the actual value and the desired value are predefined by the ventilator (Flow/Volume, possibly Pmus/FlowMus). Another possibility of combination is a stimulator and a sensor unit. The actual value is predefined here by the sensor unit and the desired value by the stimulator (activation signal). In a combination of a ventilator, stimulator and sensor unit, the actual value is predefined by the ventilator (possibly sensor unit) and the desired value by the ventilator (Pmus/FlowMus).

The different configurations of the exemplary embodiments were described in the above description mainly on the basis of the device. The process may be configured corresponding to the device. The process may thus comprise a determination of the first information and of the second information based on an airway flow measurement and on an airway pressure measurement. The indicator of the breathing assistance may comprise an indicator of the pneumatic breathing assistance. Sedation of the patient based on the indicator of the breathing assistance may also take place at least in some exemplary embodiments. Measurement information on an airway flow measurement or an airway pressure measurement may be obtained at the patient, and the indicator of the breathing assistance can further be determined on the basis of the measurement information.

The process may comprise, furthermore, a sensor-based detection of a signal, which depends on the actual respiratory muscle activation, and a determination of the indicator of the breathing assistance on the basis of the sensor-detected signal. The sensor-detected signal may comprise an electromyogram, a mechanomyogram or an electrical impedance myogram. Moreover, detection of the sensor signal may be carried out with a strain sensor, an ultrasound sensor or an esophageal pressure sensor. In addition or as an alternative, the respiratory muscles of the patient can be stimulated on the basis of the indicator of the breathing assistance. A reduction of a difference between the first information and the second information can thus also take place by control by means of the indicator of the breathing assistance.

The process may comprise in some exemplary embodiments the generation of a stimulation signal for the patient as an indicator of the breathing assistance. The first information and the second information may comprise each an indicator of a patient-side, stimulated or total respiratory muscle activation, a patient-side, stimulated or total respiratory muscle flow or a patient-side, stimulated or total respiratory muscle pressure. Moreover, information can be determined and provided on a respiratory muscle activation caused by spontaneous breathing activity of the patient, on a respiratory muscle flow caused by spontaneous breathing activity of the patient or on a respiratory muscle pressure caused by spontaneous breathing activity of the patient. The indicator of the breathing assistance can be determined on the basis of the information on the spontaneous breathing activity of the patient. The indicator of the breathing assistance can indicate an indicator of a more intensive sedation of the patient when the information on the spontaneous breathing activity of the patient indicates a respiratory muscle activity above the desired respiratory muscle activation. Further, an estimation of a stimulation pulse response of the patient can be carried out from the second information in response to the indicator of the breathing assistance. The estimation may be carried out, for example, on the basis of a stimulation maneuver, possibly also at regular intervals or continuously. Moreover, a respective reliability can be determined in some exemplary embodiments for the first information and for the second information and it can be indicated when the reliability drops below a predefined threshold.

Exemplary embodiments also create a process for a ventilator, which comprises one of the processes. A communication of information on the indicator of the breathing assistance can then take place to a stimulator. The indicator of the breathing assistance may comprise at least a parameter from the group comprising an amplitude, a ramp slope, a stimulation duration, a start time, an actual respiratory muscle activation, an end time and the desired respiratory muscle activation. The communication may take place in real time with a stimulator and/or with a sensor unit. For example, the communication takes place sample by sample in real time with a stimulator and/or with a sensor unit. The communication may comprise a communication of a time course of the indicator of the breathing assistance with a stimulator and/or with a sensor unit. The process may also comprise the predefinition of a cycle for a stimulator at least in some exemplary embodiments. Sample by sample means that the communication takes place such that it is oriented on the sequence of sample values, i.e., sample value for sample value.

The first information and the second information may be predefined for a stimulator and/or a coordination of a ventilation maneuver with a stimulator may be carried out.

Exemplary embodiments also create a process for a stimulator, which comprises one of the processes. The first information or the second information can be received from a sensor unit or from a ventilator. Moreover, at least one piece of information from the group comprising an amplitude, a ramp slope, a stimulation duration, a start time, an end time, an actual respiratory muscle activation, the desired respiratory muscle activation, an airway flow, an airway pressure, a respiratory muscle pressure, a respiratory muscle action and a respiratory muscle flow can be received from a sensor unit. A cycle can also be received in some exemplary embodiments from a sensor unit and/or from a ventilator. In addition or as an alternative, the cycle may be predefined for a sensor unit and/or for a ventilator. Communication may take place in real time. Ventilation maneuvers can be coordinated with a ventilator.

Exemplary embodiments also create a process for a sensor unit with a process. The process may comprise a receipt of information on at least one pneumatic signal from a ventilator and a determination of the indicator of the breathing assistance, also based on the information on the at least one pneumatic signal. The second information may be determined, for example, on the basis of sensor signals. Communication may also take place in real time in at least some exemplary embodiments here as well. Moreover, information can be provided to a ventilator or to a stimulator, for example, at least one piece of information from the group comprising an amplitude, a ramp slope, a stimulation duration, a start time, an end time, an actual respiratory muscle activation, an airway flow, an airway pressure, a respiratory muscle pressure, a respiratory muscle action and a respiratory muscle flow, as an indicator of the breathing assistance. Information on a maneuver can be received as well. The process may further comprise a receipt of information on a gating, measurement times to be blanked out, a filtering or a suppression of stimulation artifacts from a ventilator or from a stimulator as or with the first information.

Exemplary embodiments also create a process for a ventilation system with a process as described here. A process for a ventilator may comprise a process for a stimulator and/or a process for a sensor unit.

Exemplary embodiments can allow muscle stimulation proportionally to the breathing effort. In analogy to a proportional ventilation, a stimulation of the respiratory muscles may also take place proportionally to the spontaneous breathing of the patient. According to exemplary embodiments, information on a time course of an activation signal of the respiratory muscles of the patient is detected. At least in some exemplary embodiments, stimulation can be carried out with this information and with a factor alpha or k\hat{ }−1. The component contributed by the patient is important, for example, even if an overarching control of the degree of the positive feedback alpha takes place. In at least some exemplary embodiments, alpha can also be selected in a simple manner, e.g., 0.5. Half of the stimulation would then be added once again to the spontaneous patient activity. To detect the information on the time course of the activation signal, the control unit 14 can detect information on a time course of a component of the work of breathing that is contributed by the patient themself. Based on the information on the time course of the component of the work of breathing contributed by the patient themself, it is possible to carry out the determination of the activation signal for the respiratory muscles of the patient. The control unit 14 may be configured in this case to determine and to take into consideration a lower activation threshold (Istimoffs) for the stimulation, and an activation of the respiratory muscles takes place in case of a stimulation of the respiratory muscles above the activation threshold and an activation is at least reduced or omitted in case of a stimulation of the respiratory muscles below the activation threshold. This will still be explained below in more detail in additional figures. For example, the control unit is further configured to carry out the stimulation 24 in a positive feedback with the activation signal. A positive feedback is used, i.e., the spontaneously performed breathing effort is assisted synchronously by stimulation in case of this especially physiological type of stimulation. The assistance does not take place by the machine eliminating the work of breathing performed by the patient (as it happens during the breathing assistance by a ventilator), but by an additional muscle activation being brought about, which leads to an intensified contraction of the respiratory muscles of the patient. Even though the term “work of breathing” is used, this does not imply exclusively the physical work, but—depending on the context—the pressure applied or the volume achieved. Whenever a pressure is applied and a volume is achieved thereby, work of breathing is performed as well. No physical work is performed only in case of isometric contraction (i.e., when the volume does not change). This case occurs during occlusions, which are not being considered here. The following explanations are possible in exemplary embodiments:
The setting of the degree of positive feedback is carried out absolutely by a setter, which links the intensity of the stimulation with the amplitude of the activation or with an indicator of the breathing effort. The stimulating effect is not predictable, and titration is therefore necessary during the setting. The control unit 14 is in this case configured to determine the stimulating effect. The control unit 14 may further be configured to carry out a titration to determine the stimulating effect.
The degree of the positive feedback is set relatively, e.g., by stating a percentage. This means that the stimulating effect is related to a reference (activation signal or breathing effort) and is predictable as a result. The control unit 14 is then configured to carry out the stimulation proportionally to the activation signal.
The muscle stimulation is carried out simultaneously with the breathing assistance, which is preferably a proportional ventilation. While the proportional ventilation performs a mechanical assistance during the work of breathing, the muscle stimulation leads to an intensification of the activation. As a result, the patient-side component of the work of breathing, which is due to the muscles, increases. The control unit 14 is then configured to ventilate the patient simultaneously pneumatically. The simultaneous pneumatic proportional ventilation is carried out.
The degree of the positive feedback is set such that an overarching goal is taken into consideration. This is preferably the setting of the patient-side and machine-side components of the work of breathing. The control unit 14 is then configured, for example, to set the intensity of the stimulation on the basis of a predefined degree.

The stimulation of the respiratory muscles can be carried out proportionally to an indicator of the breathing effort. A plurality of variants are conceivable. The control unit 14 may be configured to determine information on at least one element from the group comprising
a muscle pressure, Pmus,
a spontaneous muscle pressure, Pspon,
a breathing gas flow caused by the muscles of the patient, Flowmus,
a breathing gas flow caused spontaneously by the muscles of the patient, Flowspon,
a work of breathing performed by the patient themself, WOB, and a spontaneous work of breathing performed by the patient themself, WOBspon
   as information on the time course of the activation signal of the respiratory muscles of the patient. These variables are equivalent insofar as information on the intrinsic activity of the patient can be obtained from all these. The electrical stimulation of the skeletal muscles, which depends on an activation signal, is known within the framework of the neurological rehabilitation, and so is the stimulation, which is proportional to it [E35, E36]. It is more difficult to control a magnetic stimulation than an electrical stimulation, because very high currents (>1,000 A), which must be supplied with a high frequency (interpulse period 20 msec to 100 msec, preferably 40 msec to 50 msec), flow in the stimulating coils. Both the detection of the activation of the respiratory muscles and the stimulation are more difficult because of the anatomy, the blind control and the central function of breathing than in case of the skeletal muscles. The control unit 14 is configured in some exemplary embodiments to carry out the detection of the information on the time course of the activation signal of the respiratory muscles of the patient on the basis of an electromyographic signal, of a signal from an electrical impedance myography, of a signal of a strain sensor, of a signal of an ultrasound sensor or of a mechanomyographic signal.

The relationship between the pneumatic ventilation signals, the muscle activation (EMG) and the muscle stimulation can be described by the following kinetic equation:

$$Pvent ( t ) = R \cdot Flow ( t ) + E \cdot Vol ( t ) + const - NME \cdot ( EMGspon ( t ) + EMGstim ( t ) ) ,$$

in which

$$Pmus ( t ) = NME \cdot ( EMGspon ( t ) + EMGstim ( t ) )$$

is assumed, and

$$EMGstim ( t ) = k ( t ) * Istim ( t )$$

is assumed for the activation signal caused by the stimulation (the symbol “*” is a convolution symbol).

### Variant A

The degree of the positive feedback is set in some exemplary embodiments absolutely by a setter (via a parameter a or b), which links the intensity of the stimulation with the amplitude of the activation

$$Istim ( t ) = a \cdot EMG ( t )$$

or with an indicator of the breathing effort

$$Istim ( t ) = b \cdot Pmus ( t ) .$$

The control unit 14 is then configured to determine the predefined degree by means of a ratio of the stimulation intensity to the activation signal or of the stimulation intensity to the estimated breathing effort, Pmus. The stimulating effect, i.e., the activation or the muscle pressure that it generates, is thus not predictable. Titration for the fitting setting of the factor is therefore necessary. This means that “testing” is carried out initially with low stimulation intensities and then with adapted, rather small-increment changes, to determine the stimulation intensity with which the desired activation or muscle pressure can be reached. The activation signal EMG(t) is measured, and the muscle pressure Pmus(t) is estimated [E16].

### Variant B

A relative setting is performed in some exemplary embodiments of the stimulation proportional to the breathing effort since it pertains to the activation signal. The positive feedback is:

$$EMGstim ( t ) = α \cdot EMGspon ( t ) .$$

This requirement means that the activation elicited by stimulation shall be higher by a factor α than the activation by spontaneous breathing. The degree then corresponds to a ratio between a stimulated work of breathing and a spontaneous breathing activity of the patient. Because

$$EMG ( t ) = EMGspon ( t ) + EMPstim ( t ) , \\ EMGstim ( t ) = α / ( 1 - α ) \cdot EMG ( t )$$

is obtained,
wherein α/(1−α)=β can be written for simplicity's sake.

$$EMGstim ( t ) = β \cdot EMG ( t ) .$$

The positive feed can consequently be set based on the factor α relative to the spontaneous activity or based on the factor β relative to the total activity. The latter can be achieved more easily because it is not necessary to determine first the spontaneous component of the activation. To obtain the curve of the stimulation intensity, a convolution is necessary, which can be solved, e.g., in a matrix notation by inversion of the Toeplitz matrix K [E29]. The representation of the time course of the stimulation intensity as a vector “I”^{→}_“stim” is tantamount to the sampling of the time signal Istim(t) at discrete times: t=tₖ=k·Δt.

$${\vec{I}}_{stim} = {K}^{- 1} \cdot {\vec{EMG}}_{stim} = {K}^{- 1} \cdot β \cdot \vec{EMG} .$$

As a result, the EMG signal is weighted and is used for the stimulation filtered with K⁻¹. Consequently, the activation signal EMG(t) and the (inverted) activatability k(t) are necessary for the stimulation. The latter describes in the sense of a weighting function the activation response of the muscle to a stimulation pulse. The control unit 14 can accordingly be configured to determine the spontaneous breathing activity of the patient on the basis of the information on the time course of the activation signal of the respiratory muscles of the patient. The stimulation signal can be determined in this case on the basis of the ratio of the stimulated breathing activity of the patient to an activation pulse response of the muscles of the patient. The control unit 14 can further be configured to determine the stimulation signal by inverse convolution of a desired activation signal elicited by stimulation, EMGstim, with the activation pulse response.

### Variant C

This third variant in exemplary embodiments is similar to variant B, but it pertains to the muscle pressure signal. The positive feedback is

$$Pstim ( t ) = α \cdot Pspon ( t ) .$$

This requirement means that the muscle pressure elicited by stimulation shall be higher by the factor α than the muscle pressure due to spontaneous breathing. The control unit 14 is configured here to determine the spontaneous breathing activity as a respiratory muscle pressure generated by the patient, Pspon, and the stimulated breathing activity as a respiratory muscle pressure generated by stimulation, Pstim. Because

$$Pmus ( t ) = Pspon ( t ) + Pstim ( t ) , \\ Pstim ( t ) = β \cdot Pmus ( t ) is obtained ,$$

and, taking the efficiency indicator into consideration,

$$NMEstim ( t ) \cdot EMGstim ( t ) = β \cdot NME \cdot EMG ( t ) .$$

When the efficiency indicator during stimulation does not differ from that obtained with spontaneous breathing, the result is identical to that in variant B. Otherwise,

$${\vec{I}}_{stim} = {K}^{- 1} \cdot β \cdot \frac{\vec{EMG} NME}{{NME}_{stim}} .$$

In addition to the activation signal EMG(t), the (inverted) activatability k(t) is necessary for the stimulation, along with the efficiency indicators NME and NMEstim.

### Variant D

This variant pertains to the volume generated by the muscle effort. The control unit 14 is configured here to determine the spontaneous breathing activity as a breathing gas flow generated spontaneously by the patient, Flowspon, and the stimulated breathing activity as a breathing gas flow generated by stimulation, Flowstim. The positive feedback is:

$$FlowStim ( t ) = α \cdot FlowSpon ( t ) .$$

No integral variables such as volume are used here, but the derivative thereof (Flow), because the proportional assistance is carried out in real time, rather than breath by breath. The flow caused by the muscles can in this case be calculated from the muscle pressure:

$$FlowStim ( t ) = Pstim ( t ) * DT 1 ( t )$$

$$and$$

$$FlowSpon ( t ) = Pspon ( t ) * DT 1 ( t ) ,$$

wherein DT1(t) represents the DT1 transmission element (first-order time element), which is characterized by the lung-mechanical properties of the patient. in this case,

$$Pstim ( t ) = α \cdot Pspon ( t ) is obtained ,$$

so that this variant does not differ as a result from variant C.

### Variant E

This variant pertains to the muscle work of breathing. The control unit 14 is configured here to determine the spontaneous breathing activity as a work of breathing generated spontaneously by the patient, WOBspon, or as the time derivative thereof, dWOBspon/dt, and the stimulated breathing activity as a work of breathing generated by stimulation, WOBstim, or as a time derivative thereof, dWOBstim/dt. The positive feedback is:

$$dWOBStim ( t ) dt = α \cdot dWOBSpon ( t ) dt .$$

As in variant D, the derivative is used here because of the real-time requirement. This is synonymous with:

$${P}_{stim} ( t ) Flow ( t ) = α \cdot {P}_{spon} ( t ) Flow ( t )$$

and since the flow (t) occurs on both sides of the equation, the result does not differ from variant C.

### Variant F

In this variant, which is based on one of the above variants (A through E), the degree of the positive feedback is controlled on a coarser time plane such that an overarching goal is obtained (for example, not on the sample level, but rather from breath to breath or even over a minute or longer). The control unit 14 is in this case configured to adjust the predefined degree on a time plane, which is larger than or equal to a breathing cycle of the patient.

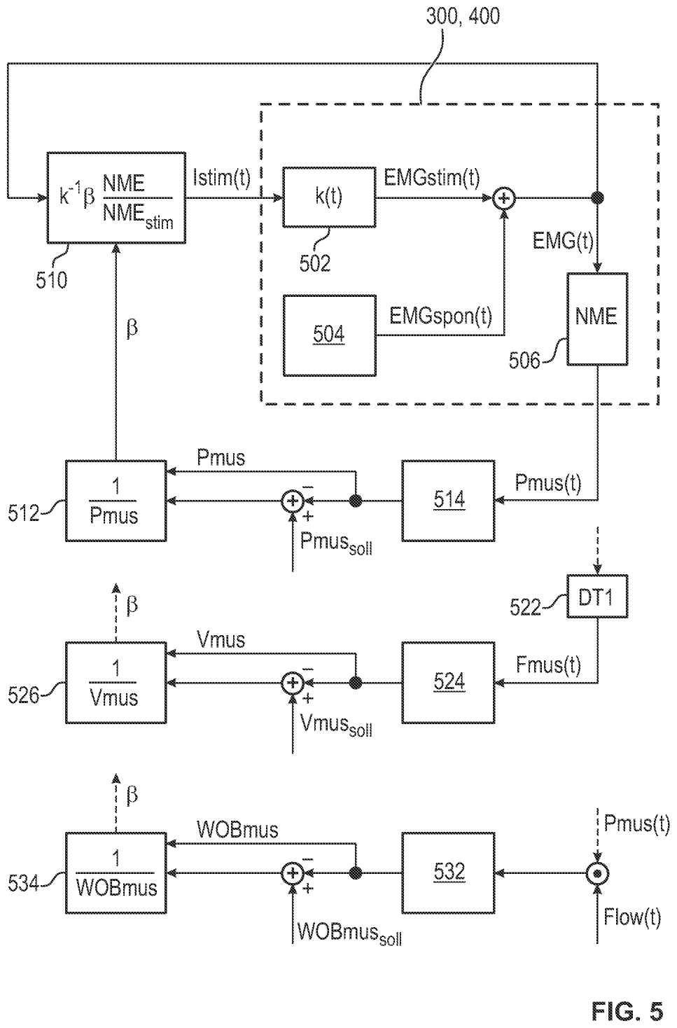

FIG. 5 shows a control in an exemplary embodiment based on a schematic diagram showing a variant. The variant shown is described based on variant F (FIG. 4). Identical elements in FIGS. 4, 5 and 11 are designated by the same reference numbers in FIGS. 4, 5 and 11. A stimulation signal Istim(t) is sent to the patient 300 or to the patient model 400, which leads to an EMG signal EMGstim(t) based on the stimulation. An EMG signal EMGspon(t) generated spontaneously by the respiratory system 504 is superimposed to this signal and leads to the total EMG signal EMG(t). This is, in turn, transformed via the NME in block 506 into a muscle pressure Pmus(t) and/or into a muscle-volume flow Fmus(t). There are two feedback loops in this case. The EMG time signal EMG(t) is returned as positive feedback, sample by sample, in order to generate proportionally to it the stimulation signal Istim(t) with block 510. It should be borne in mind in this connection that this is an amplitude-modulated pulse frequency with fixed interpulse period. The degree of positive feedback is determined essentially by β. This is determined by a second feedback depending on the deviation of the muscle pressure Pmus from the desired value Pmusₛₒₗₗ on a larger time plane in block 512 (multiplication of the control deviation by 1/Pmus). Averaging of Pmus(t) is carried out for this purpose in block 514.

Two other exemplary embodiments are shown as an alternative, the feedback being determined as a function of the deviation of the volume generated by the muscles Vmus(t) and the work of breathing WOBmus(t). The volume Vmus(t) generated by the muscles can also be calculated directly from the EMG multiplied by NVE. As is shown in this figure, the signal Fmus(t)/Flow(t), from which the volume Vmus can be determined by integration in block 524, can be generated from Pmus(t) by means of the DT1 element 522 (first-order delay element). Then, β can be determined from the difference between Vmus and Vmusₛₒₗₗ by integration in block 526 (multiplication of the control deviation by 1/Vmus). As an alternative, the product of Pmus(t) and Flow(t) can also be integrated into WOBmus(t) by means of block 532. In block 534 (multiplication of the control deviation by 1/WOBmus), β can, in turn, be determined from the difference between WOBmus and WOBmusₛₒₗₗ. The degree of the positive feedback is controlled in variant F on a rougher time plane such that an overarching goal is reached. Aside from the amplitude (which is determined directly by the degree of the positive feedback), the time course of the stimulation “I”^{→}_“stim” does not, however, change, and the proportionality to K\hat{ }(−1)·(“EMG”)^{→} continues to be present. The goal to be pursued is preferably to set the patient-side component of the driving pressure (1), at the volume (2) reached and/or at the work of breathing (3), but on a rougher time plane. The control unit 14 is configured, for example, to control the stimulation to reach a target value, which comprises a patient-side component of a driving pressure, ΔPmus, a patient-side component of a volume, ΔVolmus, or a patient-side component of a work of breathing, ΔWOBmus. The process could be embodied in a step sequence (1) through (3) similarly to the automatic setting of the proportionality factor during the proportional ventilation [E37].
(1) To control the positive feedback, a feedback of the deviation of the target value from the desired value is necessary, as it is described in [E29]. When considering the muscle pressure,

$$Δ {P}_{mus} = {Pmus}_{soll} - NME \cdot EMG$$

applies to the control deviation.

The control deviation is to be considered as an integral variable (i.e., not on the sample level), which is determined over one breath or a plurality of breaths, e.g., by averaging, determining a median or area calculation. As an alternative, the controller supplied by the control deviation carries out this calculation. The controller translates the control deviation into the manipulated variable, namely, the degree of the positive feedback, which degree is to be set. Since the control deviation shall be brought to zero by the stimulation,

$$Δ {P}_{mus} = {P}_{stim}$$

applies to the consideration and therefore

$$Pstim = β \cdot Pmus Δ {P}_{mus}$$

with the equation from variant C (which is also true integrally, i.e., on a rougher time plane).

### Resolved for β,

$$β = \frac{Δ {P}_{mus}}{{P}_{mus}} = \frac{Δ {P}_{mus}}{{P}_{{mus}_{soll}} - Δ {P}_{mus}}$$

is obtained.
(2) In a consideration of the volume generated by the muscles, the control deviation is

$$Δ {Vol}_{mus} = {Vol}_{{mus}_{soll}} - {Vol}_{mus} .$$

This can be equated with the stimulating effect:

$$Δ {Vol}_{mus} = {I}_{stim} \cdot K \cdot NVE = {EMG}_{stim} \cdot NVE .$$

Because of the proportionality:

$$EMGstim = β \cdot EMG , \\ β \frac{Δ {Vol}_{mus}}{{Vol}_{mus}} = \frac{Δ {Vol}_{mus}}{{Vol}_{{mus}_{soll}} - Δ {Vol}_{mus}}$$

is obtained.
(3) Correspondingly,

$$Δ {WOB}_{mus} = {WOB}_{{mus}_{soll}} - {WOB}_{mus}$$

applies to the control deviation.
It can be equated with the stimulating effect:

$$Δ {WOB}_{mus} = {I}_{stim} \cdot K \cdot NME \cdot Vol = {P}_{stim} \cdot Vol$$

Because of the proportionality:

$$Pstim = β \cdot Pmus$$

and because of

$${WOB}_{mus} = {P}_{mus} \cdot Vol ,$$

$$β \frac{Δ {WQOB}_{mus}}{{WOB}_{mus}} = \frac{Δ {WOB}_{mus}}{{WOB}_{{mus}_{soll}} - Δ {WOB}_{mus}}$$

is obtained.

The positive feedback can be set in all three cases on the basis of the control deviation and the desired value. A comparison of the equations from variants A and B yields the correspondences:

α=K⁻¹·β and b=a/“NME”

because of Pmus(t)=NME EMG(t), so that it is possible to set the positive feedback for all variants A through E, F (FIG. 4).

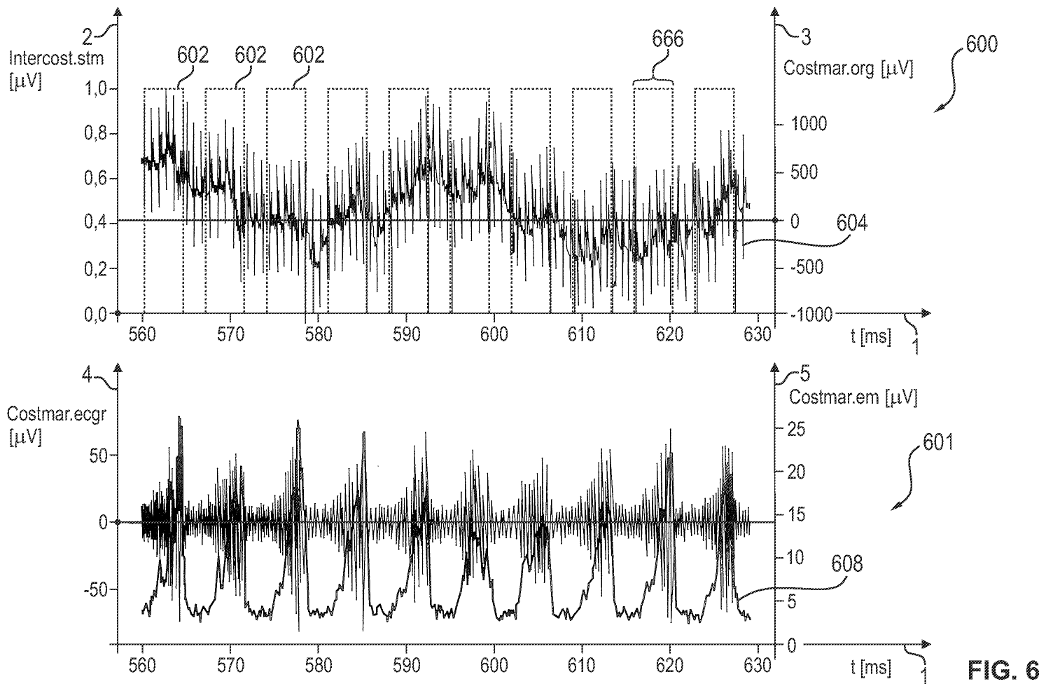

FIG. 6 shows EMG signal curves on the basis of test subject records. Abscissas (x axes) 1 are plotted as time axes with a scaling in [msec] in an upper view 600 and in a lower view 601. The signal “intercost.stm” is shown in the upper view 600 on the left side as the ordinate (y axis) 2 and the “Costmar.org” signal is shown as the ordinate (y axis) 3 on the right side. The “Costmar.ecgr” signal is shown in the lower view 601 on the left side as the ordinate (y axis) 4 and the signal “Costmar.em” is shown on the right side as the ordinate (y axis) 5. The rectangular areas 602 (“intercost.stim”) in the upper view 600 designate the time areas in which stimulation was performed (about 4.5 sec each, but not in proportion to the breathing effort). A signal “costmar.org” 604 in μV is shown in the lower view 601. The signal “costmar.org” 604 is an original EMG signal derived at the costal margin by means of two electrodes. The signal “costmar.ecgr” 606 differs from the original EMG signal 604 in that the ECG and stimulation artifacts as well as the offset were removed. The signal “costmar.emv” 608 is the envelope of the signal “costmar.ecgr” 606, formed as a mean value (root-mean-square) over a time interval of 250 msec and it is an indicator of the diaphragm activation.

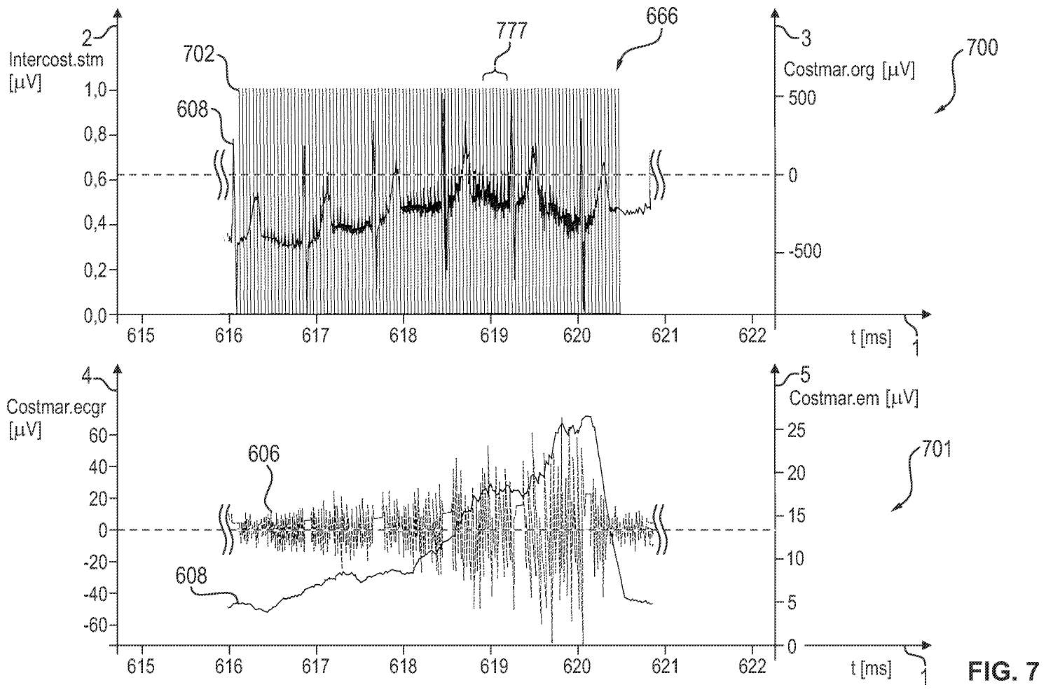

FIG. 7 shows an enlarged detail 666 from the record from FIG. 6, wherein an individual stimulated breath is shown by means of an upper view 700 and a lower view 701. Abscissas (x axes) 1 are plotted as time axes with a scaling in [msec] in an upper view 700 and in a lower view 701. The signal “intercost.stm” is shown as the ordinate (y axis) 2 in the upper view 700 on the left side and the signal “Costmar.org” is shown as the ordinate (y axis) 3 on the right side. The signal “Costmar.ecgr” is shown as the ordinate (y axis) 4 on the left side in the lower view 701 and the signal “Costmar.em” is shown as the ordinate (y axis) 5 on the right side in the lower view 701. Identical elements in FIGS. 6 and 7 are designated by the same reference numbers in FIGS. 6 and 7. The vertical gray lines 702 in the upper view 700 are the times of the stimulation pulses. As can be seen from the shape of the enveloping curve signal 608 (costmar.env), stimulation was carried out with a ramp-like intensity curve. The signal 606 shows again the curve with artifacts and offset removed (costmar.ecgr) and the signal 608 shows the corresponding envelope (costmar.env).

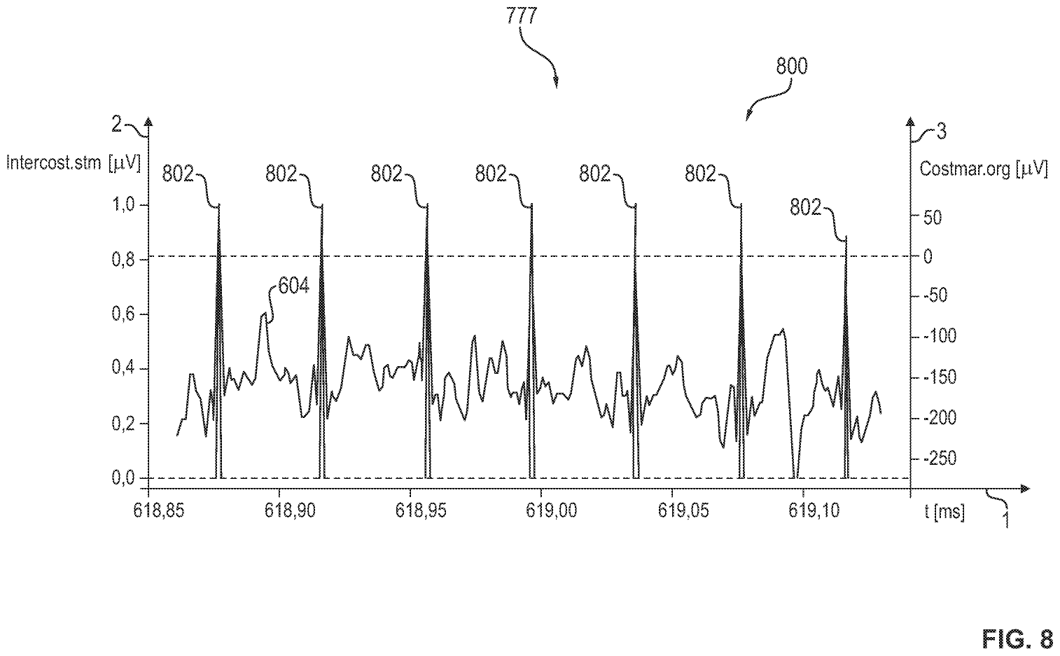

FIG. 8 shows an even more enlarged detail 777 from FIGS. 6 and 7, which comprises a number of stimulation pulses 802. The abscissa (x axis) 1 is represented as a time axis with a scaling in [msec] in a diagram 800. Identical elements in FIGS. 6, 7 and 8 are designated by the same reference numbers in FIGS. 6, 7 and 8. The signal “intercost.stm” is shown as the ordinate (y axis) 2 on the left side in the diagram 800 and the signal “Costmar.org” is shown as the ordinate (y axis) 3 on the right side. The original EMG signal 604 and the times of the stimulation pulses 802 are shown in diagram 800 analogously to the upper view 700 according to FIG. 7. About 20-30 msec after each stimulation pulse 802 the original signal 604 costmar.org has an activity pattern (stimulation responses).

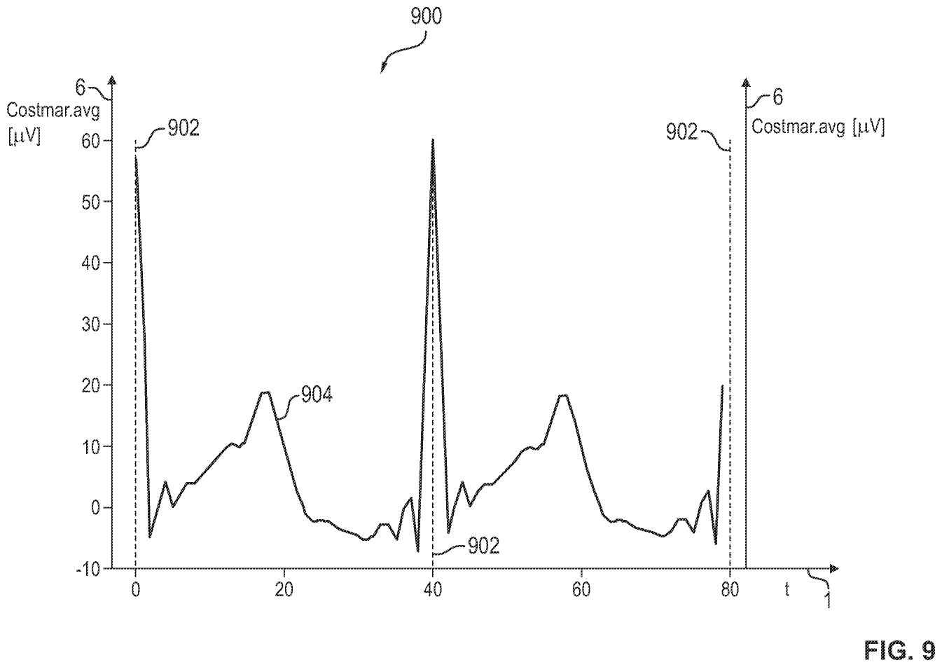

FIG. 9 shows an averaged stimulation pulse response in the time course. The abscissa (x axis) 1 is shown as a time axis. Identical elements in FIGS. 6, 7, 8 and 9 are designated by the same reference numbers in FIGS. 6, 7, 8 and 9. Stimulation responses “costmar.avg” 904 (mean original signal costmar in μV shown after the stimulation) of the original EMG signal 604 “costmar.org” (FIGS. 6, 7, 8), which stimulation responses are averaged over a broad time range, are shown here on the ordinate (y axis) 6. The averaging was carried out relative to the stimulation time and it contains two stimulation interpulse periods. The times of the stimulation pulses are indicated by the vertical broken lines 902. These lines are coincident with corresponding stimulation artifacts. Some efficiency indicators and maneuvers, which are used in exemplary embodiments, will be explained below. Efficiency indicators, which can be determined, as described in [E29] by means of maneuvers, are needed for the positive feedback and its control. These are primarily a twitch stimulation and an end-expiratory occlusion, which is possibly to be carried out simultaneously, for determining the activatability k(t) and the neuromechanical efficiency NME. An offset determination can be carried out in this case as follows. The maximum stimulation intensity Iₛₜᵢₘ_{_{offs}}, which does not lead to a muscle activation just yet, is to be subtracted from the original stimulation signal:

$${I}_{stim} ( t ) = {I}_{{stim}_{raw}} ( t ) - {I}_{{stim}_{offs}} .$$

The original stimulation signal can also be called “raw stimulation signal.” It is only thereby that a stimulation proportional to the breathing effort is possible when an intense non-linear distortion is avoided. The offset determination can be carried out automatically. A stimulation is carried out for this with varied stimulation intensities, e.g., a stimulation sequence in the form of a ramp or with randomly distributed amplitudes. The generated activation is then plotted, e.g., as a peak of the stimulation response against the corresponding stimulation intensity. A removal/suppression of artifacts may take place in some exemplary embodiments. The removal or at least the reduction of the stimulation artifacts in real time may be necessary, because the positive feedback could otherwise cause so-called runaways (outliers, uncontrolled resonant buildup of the signals because of the positive feedback of the stimulation artifacts). Further details of the artifact removal can be found in [E36, E38]. A type of gating is frequently carried out, during which the stimulation artifacts are cut off before the following filter stages in order to avoid interfering pulse responses and to lose as little signal performance as possible. Artifact reduction is also possible at the signal processing level, e.g., by ICA (independent component analysis) or wavelet transformation. Exemplary embodiments can create a stimulation intensity proportional to the breathing effort of the patient. An automatic titration of the offset (maximum stimulation intensity without activation) and/or a performance of maneuvers can take place, for example, to determine efficiency indicators and the activatability. It is possible to use a positive feedback here. A simultaneous stimulation and detection of the muscle activation can take place, and removal of the stimulation artifacts can be carried out in real time. An absolute setting of the stimulation intensity can take place in some exemplary embodiments. A relative setting can also be carried out in some exemplary embodiments, so that the clinical staff have only to state the degree of the positive feedback. An overarching goal (patient-side component of the driving pressure, volume or work of breathing) can be predefined in exemplary embodiments and an automatic setting of the positive feedback can thus be achieved. The phrenic nerve is normally stimulated with the goal of activating the diaphragm. The time course k(t) of the activation signal (by EMG) is then detected for the determination of the activatability. To obtain a reliable weighting function (kernel, pulse response), repetition of the maneuver and a corresponding averaging of the time course/time course (peristimulus-time histogram, PSTH) is advisable. The time response of the muscle activation (EMG) to a sequence of stimulation pulses can only be predicted by means of convolution with k(t). The possibly varied intensity of the stimulation pulses is possibly to be taken into consideration in this case. The component (offset) that does not lead to any activation must be subtracted basically from the stimulation intensity. Activation takes place only when the minimal activation threshold is exceeded. Linearity and the validity of the superimposition principle can (but does not obligatorily have to) be assumed thereafter. The control unit may generally be configured to determine a respiratory muscle pressure that can be generated by stimulation, Pstim, a tidal volume that can be generated by stimulation, Volstim, and/or a work of breathing of the patient, which can be generated by stimulation.

### Maneuver for Stimulating the Neuromechanical Efficiency NME During Stimulation

This maneuver can be carried out analogously to the determination of the neuromechanical efficiency (NME) during spontaneous breathing, stimulating complete breaths instead of the spontaneous breathing. It is consequently a combination of maneuvers: A breath stimulation and at the same time an end-expiratory occlusion.
Breath stimulation
A sequence of twitch maneuvers weighted in terms of their intensity is performed, so that a sequence of stimulation pulses is formed, which has a similar effect as a spontaneous breath. In other words, the duration of the sequence and possibly the shape are adapted to a spontaneous breath [E18]. The intensity of the individual pulses is normally markedly lower than the 100% intensity of a twitch pulse.
End-expiratory occlusion maneuver, determination of the neuromechanical efficiency (NME) during spontaneous breathing.
This (rather invasive) maneuver may be eliminated when the breath stimulation generates a marked activation, which can be separated from the spontaneous breathing. Just like in case of spontaneous breathing, it will then be possible by means of estimation processes to calculate NMEstim [E15].

### Maneuver for Determining the Neuroventilatory Efficiency NVE During Stimulation:

The assistance is omitted here for one or more stimulated breaths and the tidal volume generated (possibly weighted over a plurality of breaths), VolStim, is detected and related to an indicator (mean value, area, etc.) of the activation signal (EMGstim).
A combination of two maneuvers is necessary for this: The breath stimulation (see above) and the omission of the assistance in a scenario in which breathing assistance is normally performed.
Just like in the case of the determination of the neuroventilatory efficiency, a surrogate can be calculated and used for the NVE even without omission of the assistance as a quotient of VolStim and EMGstim.

### Maneuver for Determining the Stimulative Mechanical Efficiency SME(t)

A twitch stimulation is performed in this case, just like for the determination of k(t) or SVE(t), and an occlusion is performed at the same time [E21]. The time course (time curve) of the mouth closing pressure (mouth pressure) is detected and possibly averaged over a plurality of repetitions of the double maneuver.
The time response of the mouth closing pressure (which corresponds to Pmus in case of patent airways) to a sequence of stimulation pulses can be predicted in this case by means of convolution with SME(t).
As an alternative, k(t) and NMEstim can be determined one after another, instead of with a double maneuver, and the stimulative mechanical efficiency is calculated as SME(t)=k(t) NMEstim.
The amplitude of the mouth closing pressure corresponds at maximum stimulation intensity to PmusMax, which is an indicator of the maximum possible breathing effort [E11, E23].

### Maneuver for Determining the Stimulative Ventilatory Efficiency SVE(t)

A twitch stimulation is performed in this case, as for the determination of k(t) or SME(t), and the volume generated is detected at the same time without assistance by a ventilator. Normally only the pressure is detected in the literature, but the volume is not detected. The volume is possibly averaged over several repetitions of the maneuver.
The time response of the volume generated to a sequence of stimulation pulses can be predicted in this case by means of convolution with SVE(t).
As an alternative, k(t) and NVEstim can be determined one after another and the stimulative ventilator efficiency can be calculated as

SVE(t)=k(t)·NVEstim.

### Maneuver for Determining the Load-Bearing Capacity LBC:

The control unit can be configured to determine, furthermore, an indicator of a load-bearing capacity of the respiratory muscles of the patient. For example, the indicator of the load-bearing capacity can be based on a relationship between a basic load, PmusBase, and a maximum possible breathing effort, PmusMax, of the patient.
The load-bearing capacity sets the basic load (i.e., the muscular effort necessary on the basis of the possibly limited breathing mechanics) with the maximum possible breathing effort. It is calculated within the framework of this description as

LBC=1−PmusBase/PmusMax,
wherein PmusMax corresponds to an indicator (mean value, amplitude, area, or the like) of the muscle pressure, which is necessary to achieve sufficient volume with optimal breathing pattern (see primary therapy goal). This value can be calculated if the parameters of the breathing mechanics are known [E30].
An indicator of the maximum possible breathing effort is the mouth closing pressure at maximum activation, which corresponds to the maneuver for determining SME(t). PmusMax is accordingly an indicator (mean value, amplitude, area, or the like) of this target signal SME(t).

Unlike hitherto available therapy devices, the system in exemplary embodiments has at the same time the protection of the lungs as well as of the respiratory muscles, especially of the diaphragm, in its focus. It is to be expected that the number of the patients affected by VILI or VIDD is reduced by the use thereof. An important physiological reason for the improvement of the therapy is that the diaphragm, depending on the load-bearing capacity, shall possibly always follow suit during the inhalation, without the lungs being damaged thereby (due to an excessively high driving pressure). The negative pressure caused by the diaphragm has, as far as the damage to the tissue is concerned, a great advantage over the positive-pressure ventilation [E25]. Different components are preferably combined with one another for embodying a system and a process that makes it possible to adequately adjust and coordinate the ventilation and the stimulation, e.g., with a view to the respiratory muscle pressure to be performed: A ventilator 470, a stimulator 460 (actuator unit) (FIG. 4) and a sensor unit 200 (FIGS. 1a, 1b, 1c, 1d). The sensor unit (e.g., sEMG amplifier) shall in this case detect the activation signal and calculate the respiratory muscle pressure by means of pneumatic information. The stimulator 460 (FIG. 4) (actuator unit) shall, by contrast, generate from the actual value and the desired value of a target variable (e.g., muscle activation, airway flow, esophageal or muscle pressure) a stimulation signal and possibly identify for this the kernel or parameter of the system pulse response. The components are connected to one another via corresponding interfaces. The control unit can accordingly be further configured to output information on the one or more state parameters via the one or more interfaces 12. The ventilator 470 has, for example, a possibility for a mechanical pressure- or volume-controlled ventilation, a triggered pressure assistance and possibly proportional assistance of the patient's effort. The ventilator 470 has a possibility for airway flow and airway pressure measurement. If no sensor unit is available, the muscle pressure signal can be calculated. However, the calculation is less accurate when it is based only on the pneumatic signals of the sensors of the ventilator. The ventilator 470 can have the possibility of changing the degree of sedation of the patient. A deeper sedation reduces, for example, the spontaneous breathing activity. An attempt is normally made to ventilate the patient with the smallest possible quantity of sedatives or relaxants. If the respiratory muscle pressure is too high and the lungs and the diaphragm may therefore be damaged, it can be reduced by the administration of a suitable quantity of sedatives or relaxants. The actuator unit 460 (stimulator) (FIG. 4) is used for stimulation (e.g., electrically, by means of ultrasound or preferably magnetically) of the respiratory muscles. The use of the actuator is preferably carried out non-invasively on the body surface, e.g., by means of stimulating electrodes or stimulating coils. The actuator can be actuated directly by predefining a time-dependent stimulation intensity Istim(t). It is, however, desirable to control the effect of the stimulation in a fine-grained manner. For example, an indicator of the activation of the muscles, the generated airway flow or preferably the muscle pressure brought about by the contraction are considered for use as the target variable. A sensor-based feedback of the target variable is necessary for this (e.g., the read EMG, EIM or MMG enveloping curve as an indicator of the muscle activation, the flow signal or the calculated muscle pressure Pmus). After predefining this time-dependent target variable, the actuator 460 (FIG. 4) then stimulates the actuator 460 (FIG. 4) such that the desired value will be reached, i.e., the measured (read) actual value agrees with the desired value as closely as possible, i.e., the stimulation takes place in such a manner that it achieves a certain effect. The stimulation intensity is adapted correspondingly (possibly continuously). The muscle activation/flow/muscle pressure caused by spontaneous breathing activity is interpreted as an error signal. This error signal shall be able to be displayed on a GUI, because it has a therapeutic or/and diagnostic value. For example, stimulation would be performed only when the predefined total patient-side muscle pressure is greater than the current patient-side muscle pressure. No stimulation would be performed in the other case, and the sedation could possibly be increased in order to reduce the error signal (the spontaneous muscle pressure).

As an alternative, a volume signal could be used as an indicator of the activation of the respiratory muscles, e.g., the component of the volume or flow that is caused by the muscle activation [E29].

The actuator unit 460 (FIG. 4) takes into consideration the signal quality of the signals, which are necessary for the stimulating effect. If the signal quality is insufficient (e.g., the signal quality of sEMG of the respiratory muscles as a prerequisite for the calculation of the muscle pressure), the attending clinical staff can be alerted to this (alarm). The muscle pressure to be actually provided by the patient could then possibly be taken over automatically from the ventilator within the framework of a pressure assistance (fallback).

A sensor unit can be used to detect a muscle activation signal, preferably an electromyogram of the diaphragm, by means of surface electrodes. The sensor unit shall function simultaneously with the stimulation. It is therefore necessary, in general, to avoid stimulation artifacts in the detected stimulation signal or to remove them quasi in real time. Furthermore, the sensor unit detects the quality of the detected muscle activation signal or of the calculated muscle pressure signal in order to be forearmed against errors and artifact effects (e.g., movement of the body, unwanted signals). The clinical staff can be notified in case of poor quality or the system can be switched over into a fallback mode.

For example, the following definitions of the efficiency indicators can be used: A corresponding efficiency indicator, e.g., the “neuromechanical efficiency” (NME), which relates the muscle pressure generated [E18] or the “neuroventilatory efficiency” (NVE), which relates the volume generated to the EMG, is needed for the action-controlled ventilation based on an activity signal (such as EMG) [E27]. When an activity signal is available, the muscle pressure (Pmus), which corresponds to the activity signal, or the volume (VolMus) can be determined (estimated) from the activity signal by means of the efficiency indicators. This also results, the other way around, in a stipulation of the assignment of components of the work of breathing, of the muscle pressure or of the volume caused by muscles. In addition to the muscular components, the components of the ventilator can be set as well, i.e., for example, the value of the assisting pressure. With a view to the assignment of components, both the muscle pressure and the volume can be generated by spontaneous activity (which can be attenuated by the administration of sedatives/relaxants) or by stimulated activity of the respiratory muscles. The efficiency indicators are preferably identical in both cases. Depending on the anatomy and the pathology, the efficiency indicators may, however, be different, so that two separate indicators must be determined in this case, e.g., NMEspon and NMEstim or NVEspon and NVEstim. In order to make possible a stipulation for the stimulation analogously to the ventilation, an indicator of the neuronal activatability of the muscles by stimulation is additionally needed. The activatability k describes the muscle activation depending on the stimulation, e.g., the time course thereof, so that the activation signal (preferably EMG) can be calculated from the stimulation signal:

$$EMGstim ( t ) = Istim ( k ) * k ( t ) .$$

By using, e.g., NME, the time course of the muscle pressure generated by the stimulation can thus be predicted:

$$Pstim ( t ) = EMGstim ( t ) \cdot NMEstim = Istim ( t ) * k ( t ) \cdot NMEstim .$$

The time course of the volume generated by the stimulation can be predicted in exactly the same manner by means of NVE:

$$VolStim ( t ) = EMGstim ( t ) \cdot NVEstim = Istim ( t ) * k ( t ) \cdot NVEstim .$$

The activatability and the efficiency indicator can be combined:

$$SME ( t ) = k ( t ) \cdot NMEstim and \\ SVE ( t ) = k ( t ) \cdot NVEstim .$$

The indicators “stimulative mechanic efficiency” (SME) and “stimulative ventilatory efficiency” (SVE) are likewise efficiency indicators. However, they do not use the EMG as a neuronal activation signal, but the muscle pressure or volume achieved by the stimulation. Unlike NME or NVE, SME(t) and SVE(t) are potentially time-dependent signals (e.g., kernels), which are to be convoluted with the time course of the stimulation intensity. The determination of the efficiency indicators may be necessary in some exemplary embodiments for a ventilation and stimulation related to the effects thereof and it represents a characterization of the control system. It relates the respective control signal to the intended effect, so that the deviation from the expected desired value is smaller than when an attempt is made without the knowledge of the control system to achieve any kind of change in the control signal with a simple algorithm or controller (cf., e.g., [E13]). Not only a scalar factor or a characteristic, but a time-dependent kernel, which corresponds to the pulse response of the control system, is normally to be taken into consideration during the stimulation. In addition, stimulation typically takes place as a sequence of weighted pulses with an interval of 20 msec to 100 msec (preferably 40 msec to 50 msec). Each pulse triggers an individual activation (twitch), but the breath-like shape of the activation signal is obtained only after the entire pulse sequence.

The maneuvers listed in Table 2 below can be used to determine the corresponding efficiency indicators (cf. also above):

TABLE 2

| | End- | Omission of | | | Variability of | Variability of |
|---|---|---|---|---|---|---|
| | expiratory | breathing | Twitch | Breath | spontaneous | breathing |
| COMBINATION | occlusion | assistance | stimulation | stimulation | breathing | assistance |
| End-expiratory | EMGspon | x | k(t) | EMGstim | EMGspon | x |
| occlusion | Pspon | | PmusMax | Pstim | Pspon | |
| | NMEspon | | SME(t) | NMEstim | NMEstim | |
| Omission of | | EMGspon | k(t) | EMGstim | EMGspon | x |
| breathing | | VolSpon | SVE(t) | VolStim | VolSpon | |
| assistance | | NVEspon | | NVEstim | NVEspon | |
| Twitch stimulation | | | k(t) | x | X | x |
| Breath stimulation | | | | EMGstim | EMG | EMG, |
| | | | | Pstim\hat{ } | Pmus\hat{ } | R\hat{ }, E\hat{ } |
| | | | | NMEstim\hat{ } | NME\hat{ } | Pmus? |
| | | | | | | NME |
| Variability of | | | | | EMGspon | EMG, |
| spontaneous | | | | | Pspon\hat{ } | R\hat{ }, E\hat{ } |
| breathing | | | | | NMEspon\hat{ } | Pmus\hat{ } |
| | | | | | | NME\hat{ } |
| Variability of | | | | | | EMG |
| breathing | | | | | | R\hat{ }, E\hat{ } |
| assistance | | | | | | |

Table 2 shows an overview of the maneuvers and the combination thereof as well as the respective variables that can be determined from them. The “\hat{ }” symbol indicates that the variables are determined by estimation, e.g., by means of regression or Kalman filter. The suffices “stim” and “spon” designate the reference to stimulation and spontaneous breathing. When a suffix is absent, the reference is the combination of both scenarios or it cannot unambiguously be assigned. When it is to be expected that the indicators do not differ, the suffices may be omitted. The invasiveness of the maneuvers, i.e., the degree of load for the patient, varies. Depending on the degree of the load, maneuvers may be performed more or less frequently. The degree of load and the acceptable frequency are shown as an example below:
Twitch stimulation: High, 1/day
End-expiratory occlusion: Medium, 24 per day
Breath stimulation: Medium, 24 per day
Omission of breathing assistance: Low, 240 per day
Variability of breathing assistance: None, unlimited.

Depending on the configuration of the device, the maneuvers are carried out by the ventilator 470 or by the stimulator 460 (FIG. 4). However, they may also be requested from the respective other device. When, for example, the stimulator 460 (FIG. 4) wants to determine the SME(t), it requests an end-expiratory occlusion maneuver from the ventilator 470. When, on the other hand, the ventilator 470 (FIG. 4) or the connected sensor unit 200 (FIGS. 1a, 1b, 1c, 1d) wants to determine Pmus and/or NME, it possibly requests the stimulator 460 (FIG. 4) to perform a breath stimulation in order to increase the variability of the signals and to make the parameter estimation (e.g., by means of regression or Kalman filter) to be more reliable. An indicator of the signal quality is preferably to be determined for each of the detected indicators. The frequency of the maneuvers shall preferably be adapted automatically to the signal quality of the measured values of the detected indicators [E15]. When, for example, VolSpon is unreliable during an omission of the assistance, this maneuver can be repeated and the measured values can be averaged—until a sufficient reliability is reached. On the other hand, the limits of the frequency shall be taken into account depending on the degree of the load. Consequently, when a maneuver must not be repeated at first any longer because of these limits, the determined indicator receives the attribute of a low signal quality and it cannot be used any longer for certain purposes (e.g., the presentation of diagnostic information) or it can be limited to a limited extent only. For the use of the efficiency indicators, these are inverted at least in some exemplary embodiments, since the stipulation of the indicators of the work of breathing/muscle pressure/volume to be generated presupposes the target variable (e.g., when predefining the desired value). Scalar efficiency indicators may also be considered to be factors. When, for example, the target variable is predefined, the control signal can be calculated in a very simple manner by the reciprocal value of the factor, e.g., from

$$Pmus = NME \cdot EMG$$

follows the possibility of predefining Pmus for the EMG connected therewith:

$$EMG = Pmus / NME ,$$

which shall be generated by spontaneous breathing or by stimulation. Pmus and EMG may represent sampled time signals, or else (integral) values per breath.

The assistance pressure of the ventilator can be predefined because of

$$\int Pmus ( t ) dt = \int Pvent ( t ) dt \cdot a 2 / b 2 \\ as \int Pmus dt = a 2 \cdot \int Pdrv dt , \int Pvent dt = b 2 \cdot \int Pdrv dt$$

in a simple manner when the calculation is not carried out on the sample plane but integral indicators are used. This can be considered to be a preferred procedure.

$$\int EMG ( t ) dt = \int Pvent ( t ) dt \cdot a 2 / b 2 / NME .$$

The muscle activation to be required from the patient (i.e., the EMG signal) can thus be derived from the predefinition of the assisting pressure and from the required components of the driving pressure (a2 and b2). Another exemplary embodiment is the predefinition of the volume to be contributed by the muscles:

$$VolMus = NVE \cdot EMG .$$

After forming the reciprocal value, the EMG linked with the VolMus predefinition is obtained as

$$EMG = VolMus / NVE .$$

When the volume is a tidal volume, an integration of the Flow and of EMG must be carried out over the breath. If it is a minute volume, then the integration is carried out, for example, over one minute. Somewhat more difficult is the application of the efficiency indicators to the stimulation if not only integral values per breath are used, but a high time resolution is required. The time course of the stimulation intensity shall in this case be inferred from the EMG signal to be requested. As was described above, an estimation of the EMG signal generated by stimulation is obtained as follows if the kernel k(t) is known:

$$EMGstim ( t ) = Istim ( t ) * k ( t ) ,$$

but the equation cannot be solved by forming the reciprocal value according to Istim(t) (the symbol “*” is the convolution symbol). Deconvolution (inverse folding, unfolding) is needed for this, e.g., the application of a Wiener filter. The convolution operation can also be described according to the matrix notation:

$${\vec{EMG}}_{stim} = K \cdot {\vec{I}}_{stim} ,$$

wherein the components of the vectors represent samples of the respective time series. K is the Toeplitz matrix belonging to the kernel k(t). The equation can be solved for Istim:

$$( {\vec{I}}_{stim} = {K}^{- 1} \cdot {\vec{EMG}}_{stim} .$$

and an estimation of the time course of the stimulation is obtained with Istim(t) as the cause of the EMG signal presumed to be known (or required). However, if only integral values per breath are used (i.e., Istim describes the scalar stimulation amplitude for the breath and EMGstim describes a similar indicator as amplitude, area, etc., of the activation signal), then the equations become simpler in the form of the equations:

$$EMGstim = Istim \cdot k \\ and Istim = EMGstim / k ,$$

so that a reciprocal value formation is sufficient instead of a matrix inversion.
Target variables can be correspondingly predefined in exemplary embodiments. The muscle pressure generated is designated here as Pmus and the volume or the flow as VolMus and FlowMus, respectively. These variables are linked with the work of breathing as follows:

$$WOB = WOBvent + WOBmus , \\ WOBmus = \int Pmus ( t ) \cdot Flow ( t ) dt \int P ( t ) \cdot FlowMus ( t ) dt , \\ WOBvent = \cdot Pvent ( t ) \cdot Flow ( t ) dt \int P ( t ) \cdot FlowVent ( t ) dt , \\ Pdrv = Pvent + Pmus , \\ Flow = FlowVent + FlowMus , \\ Vol = VolVent + VolMus .$$

If, for example, the patient contributes a component of the total work of breathing,

$$WOBmus = a 1 \cdot WOB$$

and the ventilator correspondingly contributes

$$WOBvent = b 1 \cdot WOB ,$$

the patient and the ventilator share the total work of breathing at a ratio of a1:b1. This splitting is similar (but normally not identical) to the components of driving pressure Pdrv or flow, wherein an integral indicator can be used, e.g., the area integral for the pressure:

$$\int Pmus dt = a 2 \cdot \int Pdrv dt , \\ \int Pvent dt = b 2 \cdot \int Pdrv dt ,$$

and correspondingly for the flow:

$$\int FlowMus dt = a 3 \cdot \int Flow dt , \\ \int FlowVent dt = b 3 \cdot \int Flow dt ,$$

so that

$$VolMus = a 3 \cdot Vol , \\ VolVent = b 3 \cdot vol$$

can be written in a simplified manner.

The corresponding sum of the weights ai+bi=1, i.e., e.g., a1+b1=1, is preferably obtained. In this sense, ai*100 and bi*100 represent percentage-based components. Instead of the area integral, it would also be possible to use, e.g., a quadratic norm or a similar integral indicator. The components on the part of the patient and the ventilator do not pertain to individual sample values, but to intervals, such as phases of breath (inhalation or exhalation) or to complete breaths or a plurality of breaths. As was noted, the weights ai and bi may each be different. Thus, a patient who assumes according to the calculation a larger part of the work of breathing (WOBmus) can possibly assume a smaller part of the flow (FlowMus) when the pressure is high in the time window of the flow generated by the muscles and is low in the time window of the flow generated by the ventilator. These cases are, however, of a rather theoretical nature only. The components of the flow and volume or also of the pressures are possibly to be considered more intuitively than proportional indicators of the work of breathing, because the monitoring and the setting of the volume (e.g., minute volume MV or tidal volume VT) or of the assisting pressure is of a great clinical significance.

At least some exemplary embodiments can make possible an action-controlled ventilation and stimulation. For the action-controlled therapy, the indicator of the breathing effort must be able to be determined and set in connection with the secondary therapy goal (in the sense of a control or regulation). The breathing effort made by the patient (as work of breathing, muscle pressure or flow or volume caused by the respiratory muscles), elicited whether by spontaneous activity, stimulation or both, is determined as follows:

In the kinetic equation:

$$Pvent ( t ) + Pmus ( t ) = R \cdot Flow ( t ) + E \cdot Vol ( t ) + const$$

the muscle pressure Pmus(t) is replaced with the assumption:

$$Pmus ( t ) = NME \cdot EMG ( t ) ,$$

so that the factors R, E and NME can be determined, e.g., by means of regression or Kalman filtering by minimizing the error epsilon in the estimation equation:

$$Pvent ( t ) = R \cdot Flow ( t ) + E \cdot Vol ( t ) + const - NME \cdot EMG ( t ) + epsilon .$$

The constant can be ignored in the consideration. Since NME is known in this case, the muscle pressure can be calculated as:

$$Pmus ( t ) = NME \cdot EMG ( t ) ,$$

but also as:

$$Pmus 1 ( t ) = R \cdot Flow ( t ) + E \cdot Vol ( t ) + const - Pvent ( t ) ,$$

or as a weighted combination, wherein the difference is only in the assignment of the estimation error epsilon. The first variant will be used below, without the need to understand this as a restriction. The components related to spontaneous breathing and stimulation are obtained as:

$$Pstim ( t ) = k ( t ) * Istim ( t ) \\ and Pspon ( t ) = Pmus ( t ) - Pstim ( t ) = Pmus ( t ) - k ( t ) * Istim ( t ) .$$

The work of breathing corresponding to the muscle pressure Pmus(t) is obtained as:

$${WOB}_{mus} \int {P}_{mus} ( t ) Flow ( t ) dt$$

and correspondingly for the components:

$${WOB}_{stim} \int {P}_{stim} ( t ) Flow ( t ) dt$$

$$and$$

$${WOB}_{spon} \int {P}_{spon} ( t ) Flow ( t ) dt .$$

The airway flow triggered by the muscle pressure can be calculated according to [E29] in the frequency range as:

$$FlowMus = s / E \cdot Pmus / ( 1 + R / E \cdot s ) ,$$

which corresponds to the application of a DT1 transmission element to Pmus(t). The filtering of Pmus(t) can be easily described and accomplished in the time range by corresponding differential equations or difference equations (which are better suitable for the implementation). The volume generated by the muscle pressure is identical to the time integral of the flow:

$${VOL}_{mus} \int {Flow}_{mus} ( t ) dt$$

normally over one or possibly several breaths. The components for the spontaneous breathing and stimulation require the calculation of FlowSpon(t) and FlowStim(t) as a result of the DT1 filtering of the corresponding known pressure signals Pspon(t) and Pstim(t), i.e.,

$${VOL}_{spon} \int {Flow}_{spon} ( t ) dt$$

$$and$$

$${VOL}_{stim} \int {Flow}_{stim} ( t ) dt .$$

To make it possible to set (control) the breathing effort of the patient, the control deviation is determined, and the component of the muscle pressure WOBmus, of the work of breathing WOBmus or of the volume generated, VolMus, is predefined, either directly or by the clinical user or by an automatic therapy system. The deviation of the actual value (see above) from the predefined desired value is determined. The desired value and the actual value are each either a scalar value (preferably one value per breath) or a signal with a high time resolution (frequently represented as a vector), as it is needed for a real-time control, e.g., in case of proportional assistance or proportional stimulation. Scalar variables will be assumed below, without this necessarily meaning a limitation. For the deviation of the muscle pressure,

$$Δ {P}_{mus} {P}_{{mus}_{soll}} - NME \cdot EMG$$

wherein EMG is meant to be a parameter of the EMG signal (e.g., amplitude or area). Correspondingly for the work of breathing:

$$Δ {WOB}_{mus} {WOB}_{{mus}_{soll}} - {WOB}_{mus} .$$

Here, WOBmus is the value of the integral ∫Pₘᵤₛ(t)Flow(t)dt preferably over one breath. Analogously,

$$Δ {Vol}_{mus} {Vol}_{{mus}_{soll}} - {Vol}_{mus}$$

is the volume generated by the muscles.

A control signal is generated from the control deviation in the next step. When the control deviation is positive (the patient's performance is too low), the stimulation is carried out with the intensity:

$${I}_{stim} = Δ {P}_{mus} \cdot {SME}^{- 1} .$$

If the volume generated by the muscles is predefined,

$${I}_{stim} = Δ {Vol}_{mus} \cdot {SVE}^{- 1}$$

and when the muscular work of breathing is predefined,

$${I}_{stim} \frac{Δ {WOB}_{mus}}{SME \cdot Vol}$$

in case the stimulation intensity is sufficiently constant during the breath being considered. The work of breathing generated by stimulation can be described as:

$${WOB}_{stim} \int {P}_{stim} ( t ) Flow ( t ) dt \int [ SME ( t ) * {I}_{stim} ( t ) ] Flow ( t ) dt .$$

At constant stimulation amplitude over one breath,

$${WOB}_{stim} SME \cdot {I}_{stim} ( t ) \int Flow ( t ) dt SME \cdot {I}_{stim} ( t ) \cdot Vol ,$$

so that rearrangement can be made according to Istim to obtain:

$${I}_{stim} \frac{{WOB}_{stim}}{SME \cdot Vol} .$$

When the stimulation amplitude is not constant in the breath, the result differs somewhat from the true value. However, this plays hardly any role during the control. When the control deviation is negative (the patient performs too much), there is breathing assistance. The assisting pressure (“above PEEP”) equals

$${P}_{vent} = - Δ {P}_{mus}$$

when the muscle pressure is predefined.
When the volume to be generated by the muscles is predefined,

$${P}_{vent} = - Δ {Vol}_{mus} \cdot E$$

is true.

When considering the time of the maximum volume generated by the muscles, the restoring force caused by the volume (recoil) corresponds to this value. However, when considering integral variables (e.g., mean values over the inhalation duration), then the result differs from the value, but it plays hardly any role in the control. To obtain a more accurate result, the kinetic equation

$$Pmus ( t ) = R \cdot FlowMus ( t ) + E \cdot VolMus ( t ) + const$$

would have to be integrated over the time range (preferably the inhalation duration). The result then shows dependences on the curve describing the increase in volume VolMus(t) and of the time constant tau=R/E. When the muscular work of breathing is predefined,

$${P}_{vent} - \frac{Δ {WOB}_{mus}}{Vol} ,$$

as long as Pmus(t) is sufficiently constant during the inhalation. When the curve of the signal Pmus(t) differs greatly from a constant value, it would be necessary for a more accurate calculation to calculate the integral:

$${WOB}_{mus} \int {P}_{mus} ( t ) Flow ( t ) dt$$

and to solve it for an indicator (e.g., mean value) of Pmus(t). This calculation is not trivial and is not normally needed, since the above-mentioned approximation is sufficient within the framework of the control. When the control deviation is lastingly very high (the patient performs considerably too much), the intrinsic breathing must be attenuated to protect the patient by the administration of sedatives or relaxants. This preferably happens by intervention by the clinical staff. Automation would be possible, but it would require an estimation of the sedating effect. If the activatability k(t) changes as a result, corresponding maneuvers would have to be performed in order to determine it anew. The automatic ventilation and the stimulation require the following steps in an exemplary embodiment:
Predefinition of the primary therapy goal:
   preferably minute volume (MV) or tidal volume (VT) and respiration rate (f);
Determination of the breathing-mechanical parameters:
   predominantly resistance (R) and elastance (E);
   mandatory ventilation with the patient sedated or
   ensuring sufficient variability of the assistance during spontaneous breathing (e.g., by Noisy PSV (“pressure support ventilation,” pressure-assisting ventilation mode, during which the assistance is slightly varied accidentally)) and estimation of the parameters, e.g., by means of regression or Kalman filter;
calculation of the optimal respiration rate [E30];
determination of the basic breathing load (PmusBase) depending on the primary therapy goal;
determination of the activatability and of the maximum possible respiratory muscle effort PmusMax by twitch stimulation and end-expiratory occlusion;
determination of the efficiency indicators;
determination of the load-bearing capacity LBC;
predefinition of the secondary therapy goal:
   manually by the clinical user. The basic breathing load, load-bearing capacity, breathing effort made by the patient, etc., are displayed on the GUI of the ventilator. Based on this, the clinical user can estimate the load that is reasonable for the patient. The component (work of breathing, muscle pressure or volume to be generated by the muscles) that is to be contributed by the patient is to be correspondingly predefined by means of the GUI;
   automatically by a therapy system. The component of the breathing effort to be made by the patient is set at a percentage X (e.g., 50%) of the maximum breathing effort to be made by the patient (e.g., WOBmusMax, VolmusMax or PmusMax). The therapy system seeks to request the corresponding work of breathing from the patient;
The control deviation is determined;
An adjustment of the breathing assistance or of the stimulation intensity is proposed or carried out automatically based on the control deviation. The administration of sedatives or relaxants is preferably carried out by intervention by the clinical staff.

Exemplary embodiments can in this case especially support the diagnostics. The determined values or characteristics of the
activatability,
efficiency indicators,
load-bearing capacity,
maximum possible breathing effort,
spontaneous breathing effort,
breathing effort elicited by stimulation
   have diagnostic relevance and help the clinical staff in estimating the patient's situation and the progress of the therapy.

The indicators can be displayed graphically on the GUI of the ventilator and possibly of the stimulator, for example, over time, as a bar graph, trend diagram or 2-dimensional plot (e.g., Pmus vs. Vol). If available, corresponding signal quality indicators should be taken into consideration in this case. For example, an unreliable signal would only have a weak contrast or would not be displayed at all. Scalar indicators may be displayed, e.g., in tables or so-called P boxes. Depending on the relevance for the patient and the clinical staff, messages, alarms or instructions for action can be outputted.

Exemplary embodiments can accordingly provide certain maneuvers for the determination of
activatability (characteristic that describes the ability to activate the muscles by stimulation),
efficiency (target variable, which is reached by activation of muscles, e.g., NME or NVE), and/or
load-bearing capacity (dependent on the ratio of the basic breathing load to the maximum possible breathing effort).

Different types of maneuvers, which occur in exemplary embodiments, are, for example,
Twitch stimulation,
breath stimulation,
end-expiratory occlusion,
omission of the ventilatory assistance for individual breathing cycles, and
other forms of the variability of ventilatory assistance and possibly stimulation.

The control unit may be configured to carry out these maneuvers. Graphic representation of the characteristics (activatability, efficiency, load-bearing capacity) for the diagnostics may occur in exemplary embodiments. The characteristics for the automation of the ventilation and stimulation can be used in exemplary embodiments.

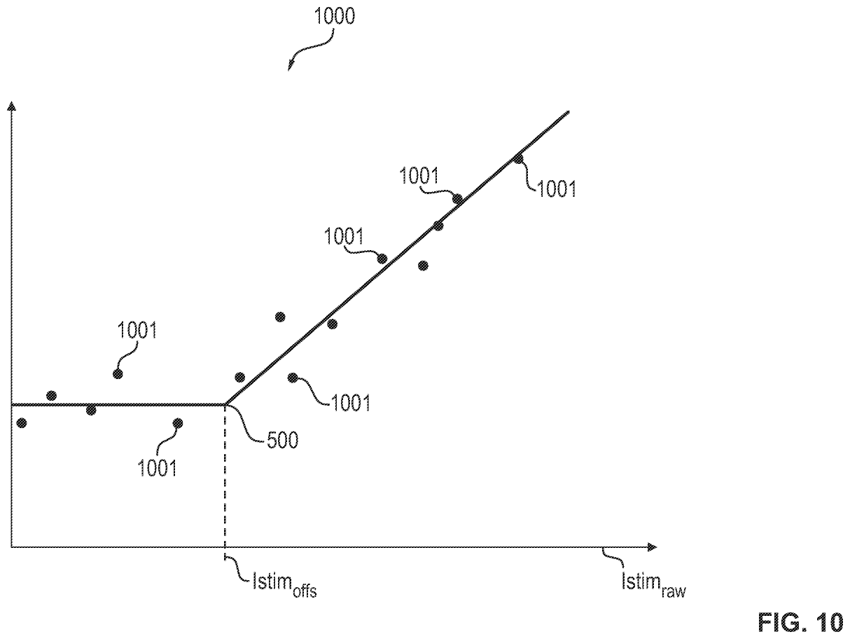

FIG. 10 shows a schematic view 1000 of a muscle activation (y-axis, ordinate) as a function of the stimulation intensity Istimr_{aw} (x axis, abscissa) in an exemplary embodiment with activation threshold. Different measuring points 1001 of the generated activation are plotted here against the corresponding stimulation intensity. The dependence of the activation on the stimulation can be quantified by means of a regression or other methods. In particular, the intensity Istim_{offs} 500 (activation threshold) can be determined at which the characteristic has a bend (kink/deflection). In the intensity Istim_{offs} 500 the characteristic has an upward bend, i.e., activation of the respiratory muscles occurs beginning from this intensity 500.

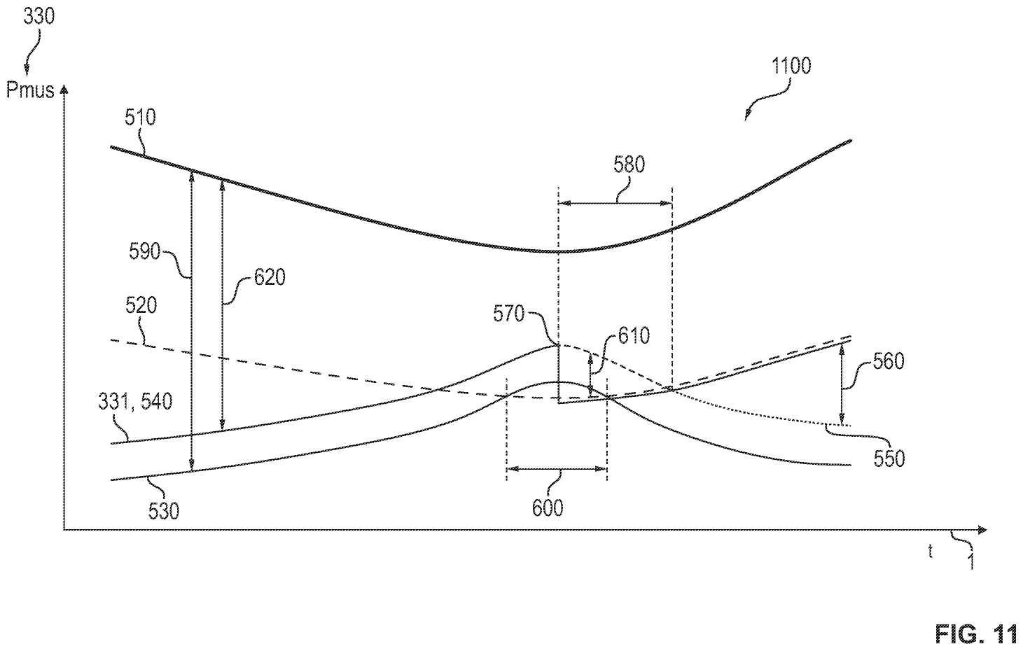

FIG. 11 shows a schematic view of a time course of a muscle pressure 331, 540, Pmus 330. The abscissa (x axis) 1 is a time axis. Identical elements in FIGS. 5 and 11 are designated by the same reference numbers in FIGS. 5 and 11. The curve 510 shows the maximum possible muscle pressure PmusMax and the curve in broken line 520 shows 50% of this maximum muscle pressure PmusMax. The curve 530 shows the basic breathing load PmusBase and the curve 540 shows the muscle pressure Pₘᵤₛ, which is composed of a spontaneous component 550 and a stimulated component 560. PmusBase 530 is the basic load, i.e., the muscle pressure that is needed to achieve a sufficient minute volume during optimal breathing rhythm. PmusMax 510 is the maximum muscle pressure that the patient can produce. 50% of PmusMax 520 is the predefined value of the breathing effort, which shall be required from the patient. Pmus 540 is the real breathing effort provided by the patient. This exceeds at time the 50% PmusMax line 520, which leads to exhaustion (point 570) and to the need for breathing assistance after a short time in the example (area 580). The load-bearing capacity 590 LBC=1−PmusBase/PmuxMax is the (relative) distance between the basic load 530 and the maximum possible breathing effort 510. It is very low in the interval characterized by broken lines (600, basic load higher than the 50% mark 520, there is a threatening exhaustion) and explains the fatigue. An attempt is made for the automation to maintain Pmus 540 in the vicinity of the 50% PmusMax line 520. If Pmus is lower, then stimulation (560) is performed. If Pmus is higher (especially when the basic load rises), breathing assistance is given, in this example with the assisting pressure 610. A reverse 620 is obtained between the actual Pmus and the maximum possible muscle pressure PmusMax. Hyperventilation—either by an excessively high assisting pressure or by an excessively high stimulation intensity—can be manifested by periodic breathing. The sEMG of the respiratory muscles shows in this case a periodic modulation with a time constant on the order of magnitude of one minute or more. The periodic breathing is also reflected by the flow and respiratory muscle signal. For example, the EMG signal drops after an increase in the assisting pressure Paw to a low value to rise and drop then again briefly periodically (apnea). When a periodic breathing is detected, the assisting pressure and/or the stimulation intensity should be reduced for a trial. If the periodic breathing intensity decreases, there obviously was a hyperventilation. If not, the assisting pressure/the stimulation intensity is set at the previous value. The synchronization of the stimulation takes into account both the start and the end of the breaths. It would thus be possible, for example, to transfer a protective or energy-optimized timing of the ventilation pattern directly to the stimulation signal [E30, E33, E34]. This also appears to be useful when no mechanical ventilation, but only an assistance takes place. The pattern of the stimulation intensity is selected in a lung-protective and energy-optimized manner as long as a possible spontaneous activity is synchronized with it. It is possible and also probable that the patient agrees to a lung-protective and energy-optimized stimulation pattern and synchronizes the patient's spontaneous activity with it. If this does not happen within a predefined time period, the spontaneous activity pattern of the patient is taken over as the basis for the stimulation.

The aspects and features that are described together with one or more of the examples and figures described in detail before may also be combined with one or more of the other examples in order to replace an identical feature of the other example or to additionally introduce the feature into the other example. Examples may be or pertain to, furthermore, a computer program with a program code for carrying out one or more of the above processes when the computer program is executed on a computer or processor. Steps, operations or processes of different processes described above can be executed by programmed computers or processors. Examples may also cover program memory devices, e.g., digital storage media, which are machine-, processor- or computer-readable and code machine-executable, processor-executable or computer-executable programs of instructions. The instructions execute some or all of the steps of the above-described processes or cause them to be executed. The program memory devices may comprise or be, e.g., digital storage devices, magnetic storage media, for example, magnetic disks and magnetic tapes, hard drives or optically readable digital storage media. Other examples may also cover computers, processors or control units, which are programmed to execute the steps of the above-described processes, or (field)-programmable logic arrays ((F)PLAs=(Field) Programmable Logic Arrays) or (field)-programmable gate arrays ((F)PGA=(Field) Programmable Gate Arrays), which are programmed to execute the steps of the above-described processes. Only the principles of the disclosure are described by the description and drawings. Furthermore, all the examples mentioned here shall serve basically expressly only illustrative purposes in order to support the reader in understanding the principles of the disclosure and concepts contributed by the inventor(s) to the further development of the technique. All statements made here about principles, aspects and examples of the disclosure as well as concrete examples thereof comprise their equivalents. A function block designated as a “means for . . . ” performing a defined function may pertain to a circuit, which is configured to perform a certain function. Thus, a “means for something” may be implemented as a “means configured for or suitable for something,” e.g. a component or a circuit configured or suitable for the particular object. Functions of different elements shown in the figures, including each function block designated as “means,” “means for providing a signal,” “means for generating a signal,” etc., may be implemented in the form of dedicated hardware, e.g., of “a signal provider,” of “a signal processing unit,” of “a processor,” of “a control,” etc., as well as hardware capable of executing software in connection with corresponding software. In case of provision by a processor, the functions may be provided by an individual dedicated processor, by an individual, jointly used processor or by a plurality of individual processors, some of which or all of which may be used jointly. However, the term “processor” or “control” is far from being limited to hardware capable exclusively for executing software, but it may comprise digital signal processor hardware (DSP hardware; DSP=Digital Signal Processor), network processor, application-specific integrated circuit (ASIC=Application Specific Integrated Circuit), field-programmable logic array (FPGA=Field Programmable Gate Array), read-only memory (ROM=Read Only Memory) for storing software, random access memory (RAM=Random Access Memory) and non-volatile memory device (storage). Other hardware, conventional and/or customized, may be included. A block diagram may represent, for example, a schematic circuit diagram, which implements the principles of the disclosure. Similarly, a flow chart, a flow process diagram, a state transition diagram, a pseudocode and the like may represent different processes, operations or steps, which are represented, for example, essentially in computer-readable medium and are thus executed by a computer or processor, regardless of whether such a computer or processor is explicitly shown. Processes disclosed in the description or in the patent claims may be implemented by a component, which has means for executing each and every one of the respective steps of these processes. It is apparent that the disclosure of a plurality of steps, processes, operations or functions disclosed in the description or in the claims shall not be interpreted as being configured in the specified order, unless this is stated explicitly or implicitly otherwise, e.g., for technical reasons. Therefore, these are not limited by the disclosure of a plurality of steps or functions to a defined order, unless these steps or functions are not replaceable for technical reasons. Further, an individual step, function, process or operation may include in some examples a plurality of partial steps, partial functions, partial processes or partial operations and be broken up into same. Such partial steps may be included and be a part of the disclosure of this individual step, unless they are explicitly excluded. Furthermore, the following claims are included hereby in the detailed description, where each claim may stand in itself as a separate example. While each claim may stand in itself as a separate example, it should be noted—even though a dependent claim may pertain in the claims to a defined combination with one or more other claims—other examples may also comprise a combination of the dependent claim with the subject of every other dependent or independent claim. Such combinations are proposed here explicitly, unless it is stated that a defined combination is not intended. Further, features of a claim shall also be included for every other independent claim, even if this claim is not made directly dependent on the independent claim.

A plurality of the different embodiments described above in this document and possible combinations thereof will be compiled in an overview below. These different embodiments and their possible combinations form a basis for further preferred and also especially preferred embodiments according to the present invention as well as independent and/or coordinate patent claims based on these embodiments, as well as subclaims based on advantageous embodiments.

Further and preferred embodiments of the present invention will be described in more detail below concerning a concept (device, system and process) for determining a state of respiratory muscles of a patient, especially a concept (device, system and process) for determining at least one state parameter of the respiratory muscles of a patient on the basis of an analysis of an activation signal as a response to a stimulation of the respiratory muscles. A basic embodiment shows a device for determining a state of the respiratory muscles of a patient with one or more interfaces, which are configured to detect patient signals, and with a control unit, which is configured
to stimulate the respiratory muscles of the patient with a stimulation signal,
to detect an activation signal as a response to the stimulation, and
to determine one or more state parameters for the respiratory muscles based on the stimulation signal and on the activation signal.

In a preferred embodiment based on the above-described embodiment, the control unit may be configured to generate the stimulation signal with one or more stimulation pulses.

In a preferred embodiment based on at least one of the above-described embodiments, the control unit may be configured to detect the activation signal as a pulse response.

In a preferred embodiment based on at least one of the above-described embodiments, the control unit may be configured to determine an activatability of the respiratory muscles of the patient with the determination of the one or more state parameters.

In a preferred embodiment based on at least one of the above-described embodiments, the control unit may be configured to take into consideration a lower activation threshold during the determination of the activatability, wherein activation of the respiratory muscles takes place in case of a stimulation of the respiratory muscles above the activation threshold, and activation is at least reduced or it is not performed in case of a stimulation of the respiratory muscles below the activation threshold.

In a preferred embodiment based on at least one of the above-described embodiments, the control unit may be configured to determine a respiratory muscle pressure, Pₛₜᵢₘ, which can be generated by stimulation, a tidal volume, Volₛₜᵢₘ, which can be generated by stimulation, and/or a work of breathing of the patient, which can be generated by stimulation.

In a preferred embodiment based on at least one of the above-described embodiments, the control unit may further be configured to perform a pneumatic diagnostic maneuver to determine a pneumatic ventilation parameter, and to determine the one or more state parameters on the basis of the pneumatic ventilation parameter.

In a preferred embodiment based on at least one of the above-described embodiments, the pneumatic diagnostic parameter may comprise an occlusion, a breath flow limitation, an omission of an assistance of individual breaths or a variability in the breathing assistance of the patient.

In a preferred embodiment based on at least one of the above-described embodiments, the control unit may be further configured to determine an indicator of a maximum possible breathing effort of the patient.

In a preferred embodiment based on at least one of the above-described embodiments, the indicator of the maximum possible breathing effort of the patient may comprise a mouth closing pressure at maximum activation of the respiratory muscles.

In a preferred embodiment based on at least one of the above-described embodiments, the control unit may be further configured to determine an indicator of a load-bearing capacity of the respiratory muscles of the patient.

In a preferred embodiment based on at least one of the above-described embodiments, the indicator of the load-bearing capacity may be based on a relationship between the basic load, Pmus_{Base}, and a maximum possible breathing effort, Pmus_{Max}, of the patient.

In a preferred embodiment based on at least one of the above-described embodiments, the control unit may further be configured to determine an indicator of an efficiency of the respiratory muscles of the patient.

In a preferred embodiment based on at least one of the above-described embodiments, an indicator of the efficiency may comprise a ratio of a tidal volume achievable by stimulation to the activation signal.

In a preferred embodiment based on at least one of the above-described embodiments, the indicator of the efficiency may comprise a ratio of a respiratory muscle pressure achievable by stimulation to the activation signal.

In a preferred embodiment based on at least one of the above-described embodiments, the control unit may be configured to output information on the one or more state parameters via the one or more interfaces.

A basic embodiment shows a process for determining a state of the respiratory muscles of a patient with
stimulation of the respiratory muscles of the patient with a stimulation signal,
detection of an activation signal as a response to the stimulation, and
determination of one or more state parameters for the respiratory muscles based on the stimulation signal and on the activation signal.

In a preferred embodiment based on the basic embodiment, the stimulation signal may comprise one or more stimulation pulses.

In a preferred embodiment based on at least one of the above-described embodiments, the activation signal may be detected as a pulse response.

In a preferred embodiment based on at least one of the above-described embodiments, the determination of the activatability may comprise the taking into consideration of a lower activation threshold for the stimulation signal, wherein activation of the respiratory muscles is reduced or does not take place in case of a stimulation of the respiratory muscles below the activation threshold.

In a preferred embodiment based on at least one of the above-described embodiments, the determination of the one or more state parameters may comprise a determination of an activatability of the respiratory muscles of the patient.

A preferred embodiment based on at least one of the above-described embodiments may
comprise a determination of a respiratory muscle pressure Pₛₜᵢₘ that can be generated by stimulation,
comprise a determination of a tidal volume, Volₛₜᵢₘ, which can be generated by stimulation, and
comprise a determination of a work of breathing of the patient, which can be generated by stimulation.

A preferred embodiment based on at least one of the above-described embodiments may comprise the performance of a pneumatic diagnostic maneuver to determine a pneumatic ventilation parameter and determination of one or more state parameters based on the pneumatic ventilation parameter.

In a preferred embodiment based on at least one of the above-described embodiments, the pneumatic diagnostic maneuver may comprise an occlusion, a breath flow limitation, an omission of an assistance of individual breaths or a variability in the breathing assistance of the patient.

A preferred embodiment based on at least one of the above-described embodiments may comprise a determination of an indicator of a maximum possible breathing effort of the patient.

In a preferred embodiment based on at least one of the above-described embodiments, the indicator of the maximum possible breathing effort of the patient may comprise a mouth closing pressure during maximum activation of the respiratory muscles.

A preferred embodiment based on at least one of the above-described embodiments may comprise a determination of an indicator of a load-bearing capacity of the respiratory muscles of the patient.

In a preferred embodiment based on at least one of the above-described embodiments, the indicator of the load-bearing capacity may be based on a relationship between a basic load, P_{musBase}, and a maximum possible breathing effort, P_{musMax}, of the patient.

A preferred embodiment based on at least one of the above-described embodiments may comprise a determination of an indicator of an efficiency of the respiratory muscles of the patient.

In a preferred embodiment based on at least one of the above-described embodiments, the indicator of the efficiency may comprise a ratio of a tidal volume achievable by stimulation to the activation signal.

A preferred embodiment based on at least one of the above-described embodiments may comprise the output of information on the one or more state parameters.

A basic embodiment may comprise a computer program with a program code for executing at least one of the above-described embodiments. The program code may advantageously be executed on a computer, on a processor or on a programmable hardware component.

Additional and preferred embodiments of the present invention will be described below in more detail concerning a ventilation system, a device, a process and a computer program for ventilating a patient with a concept for determining a load-bearing capacity and an indicator of a component to be contributed by the patient themself to the ventilation and for taking into consideration the load-bearing capacity and the indicator during the assistance of the ventilation. A basic embodiment includes a device for ventilating a patient with one or more interfaces, which device is configured for the exchange of information with a ventilating unit, with a stimulation unit or with a sensor unit and has a control module, which is configured
to detect an indicator of a component of the ventilation, which is to be contributed by the patient,
to determine an indicator of a load-bearing capacity of the patient,
to influence the component of the ventilation, which is contributed by the patient themself, and
to assist the patient during the ventilation based on the indicator of the component of the ventilation that is contributed by the patient themself and based on the indicator of the load-bearing capacity of the patient.

In a preferred embodiment based on the above-described embodiment, the control unit may be configured to output a signal for the assistance for the pressure-controlled or volume-controlled ventilation. In a preferred embodiment based on at least one of the above-described embodiments, the control unit may be configured to output a signal for stimulating the respiratory muscles of the patient for the assistance.

In a preferred embodiment based on at least one of the above-described embodiments, the indicator of the work performed by the patient themself may comprise at least one element from the group comprising
a muscle pressure Pₘᵤₛ, absolute or relative to a total breathing pressure P_{aw},
a total driving pressure P_{drv},
a breathing gas flow caused by the muscles of the patient, Flowₘᵤₛ, absolute or relative to a total breathing gas flow Flow,
a tidal volume caused by the muscles of the patient, Volₘᵤₛ, absolute or relative to a total tidal volume Vol,
a work of breathing contributed by the patient themself, WOBₘᵤₛ, absolute or relative to a total work of breathing WOB.

In a preferred embodiment based on at least one of the above-described embodiments, the detection of the indicator of the component contributed by the patient themself can be carried out on the basis
of an electromyographic signal,
of a signal from an electrical impedance myography,
of a mechanomyographic signal,
of an ultrasound signal,
of a signal of a strain sensor, and
of a signal of an esophageal pressure sensor.

In a preferred embodiment based on at least one of the above-described embodiments, the control module may be configured to output a signal for stimulating the respiratory muscles of the patient to influence the component contributed by the patient themself.

In a preferred embodiment based on at least one of the above-described embodiments, the control module may be configured to output a signal to influence an administration of drugs for influencing the component contributed by the patient themself.

In a preferred embodiment based on at least one of the above-described embodiments, the control module may be configured to control the ventilation of the patient concerning a ventilation parameter predefined as a primary goal.

In a preferred embodiment based on at least one of the above-described embodiments, the control module may be configured to control the component contributed by the patient themself on the basis of a component of the ventilation that is predefined as a secondary goal.

In a preferred embodiment based on at least one of the above-described embodiments, the control module may be configured to monitor and to control the entire tidal volume generated by the patient themself.

In a preferred embodiment based on at least one of the above-described embodiments, the control module may be configured to control and to monitor the work of breathing generated by the patient themself.

In a preferred embodiment based on at least one of the above-described embodiments, the control module may be configured to monitor and to control an oxygenation of the patient.

In a preferred embodiment based on at least one of the above-described embodiments, the control module may be configured to make the influencing and the assistance conform with a breathing rhythm predefined by the patient.

In a preferred embodiment based on at least one of the above-described embodiments, the control module may be configured to make the assistance conform with a breathing rhythm predefined by the influencing.

In a preferred embodiment based on at least one of the above-described embodiments, the control module may be configured to make the influencing and the assistance conform with a breathing rhythm, predefined by the patient if the spontaneous activity of the patient is present and is harmless, and to make the assistance otherwise conform with a breathing rhythm, predefined by a stimulation if a spontaneous activity of the patient is not present or is harmful and to make the assistance conform with a breathing rhythm predefined by a pneumatic ventilation if a stimulating effect fails to occur.

In a preferred embodiment based on at least one of the above-described embodiments, the control module may be configured to determine an efficiency of the respiratory muscles of the patient, wherein the assistance is further based on the efficiency.

In a preferred embodiment based on at least one of the above-described embodiments, the control module may be configured to determine an activatability of the respiratory muscles of the patient, wherein the assistance is further based on the activatability.

In a preferred embodiment based on at least one of the above-described embodiments, the control module may be configured to determine an exhaustion of the respiratory muscles of the patient, wherein the assistance is further based on the exhaustion.

In a preferred embodiment based on at least one of the above-described embodiments, the control module may be configured to determine a breathing-mechanical basic load and to determine a maximum possible breathing effort of the patient to determine the load-bearing capacity.

In a preferred embodiment based on at least one of the above-described embodiments, the control module may be configured to determine the maximum possible breathing effort by performing a twitch stimulation,

In preferred embodiments, a ventilation system for assisting a patient during the ventilation may be equipped or configured with a device based on at least one of the above-described embodiments.

A basic embodiment shows a process for ventilating a patient with
detection of an indicator of a component of the ventilation contributed by the patient themself,
determination of an indicator of a load-bearing capacity of the patient,
influencing of the component of the ventilation contributed by the patient themself,
assistance of the patient during the ventilation based on the indicator the component of the ventilation contributed by the patient themselves and on the basis of the indicator of the load-bearing capacity of the patient.

In a preferred embodiment based on the above-described embodiment, the assistance may comprise pressure-controlled or volume-controlled ventilation.

In a preferred embodiment based on at least one of the above-described embodiments, the assistance may comprise a stimulation of the respiratory muscles of the patient.

In a preferred embodiment based on at least one of the above-described embodiments, the indicator of the work contributed by the patient themself may contain at least one element from the group comprising
a muscle pressure Pₘᵤₛ, absolute or relative to a total breathing pressure P_{aw},
a total driving pressure P_{drv},
a breathing gas flow Flowₘᵤₛ caused by the muscles of the patient, absolute or relative to a total breathing gas flow,
a tidal volume Volₘᵤₛ caused by the muscles of the patient, absolute or relative to a total tidal volume Vol, and
a work of breathing WOBₘᵤₛ contributed by the patient themself, absolute or relative to a total work of breathing WOB.

In a preferred embodiment based on at least one of the above-described embodiments, the detection of the indicator of the component contributed by the patient themself can be carried out on the basis
of an electromyographic signal,
of a signal from an electrical impedance myography,
of a mechanomyographic signal,
of an ultrasound signal,
of a signal of a strain sensor, and
of a signal of an esophageal pressure sensor.

In a preferred embodiment based on at least one of the above-described embodiments, the influencing of the component contributed by the patient themself may comprise a stimulation of the respiratory muscles of the patient.

A preferred embodiment based on at least one of the above-described embodiments may further comprise a control of the ventilation of the patient concerning a ventilation parameter predefined as a primary goal.

A preferred embodiment based on at least one of the above-described embodiments may further comprise a control of the component contributed by the patient themself on the basis of a component of the ventilation that is predefined as a secondary goal.

A preferred embodiment based on at least one of the above-described embodiments may further comprise a monitoring and control of the total tidal volume of the patient and of the tidal volume of the patient that is generated by the patient themself.

A preferred embodiment based on at least one of the above-described embodiments may further comprise a monitoring and control of the work of breathing generated by the patient themself.

A preferred embodiment based on at least one of the above-described embodiments may further comprise a monitoring and control of the oxygenation of the patient.

In a preferred embodiment based on at least one of the above-described embodiments, the influencing and the assistance may be made conform to a breathing rhythm predefined by the patient.

In a preferred embodiment based on at least one of the above-described embodiments, the assistance may be made conform with a breathing rhythm predefined by the influencing.

In a preferred embodiment based on at least one of the above-described embodiments, the assistance may be made conform with a breathing rhythm predefined by the patient if the patient's spontaneous activity is present and is harmless, wherein the assistance is made otherwise conform with a breathing rhythm predefined by a stimulation if a spontaneous activity of the patient is not present or is harmful, wherein the assistance is made conform with a breathing rhythm predefined by a pneumatic ventilation if a stimulating effect does not occur.

A preferred embodiment based on at least one of the above-described embodiments may comprise a determination of an efficiency of the respiratory muscles, wherein the assistance is based further on the efficiency.

A preferred embodiment based on at least one of the above-described embodiments may further comprise a determination of an activatability of the respiratory muscles of the patient, wherein the assistance is based further on the activatability.

A preferred embodiment based on at least one of the above-described embodiments may further comprise a determination of an exhaustion of the respiratory muscles of the patient, wherein the assistance is based further on the exhaustion.

In a preferred embodiment based on at least one of the above embodiments, the determination of the load-bearing capacity may comprise a determination of a breathing-mechanical basic load and a detection of a maximum possible breathing effort of the patient.

In a preferred embodiment based on at least one of the above-described embodiments, the detection of the maximum possible breathing effort may comprise the performance of a twitch stimulation.

A basic embodiment may comprise a computer program with a program code for carrying out at least one of the above-described embodiments. The program code may advantageously be executed on a computer, on a processor or on a programmable hardware component.

Further and preferred embodiments of the present invention will be described in more detail below concerning a device, a process and a computer program for a component of a ventilation system for the breathing assistance of a patient concerning a concept for determining an indicator of a breathing assistance of a patient based on a desired respiratory muscle activation and on an actual respiratory muscle activation. Further aspects concerning a ventilator, a stimulator, a sensor unit as well as aspects pertaining to different processes and computer programs for a ventilator, a stimulator and a sensor unit, a ventilation system, a process and a computer program for a ventilation system will be described as well. A basic embodiment includes a device for a component of a ventilation system for the breathing assistance of a patient with one or more interfaces for the communication with components of the ventilation system and with a control unit, which is configured
for determining a first piece of information on a desired respiratory muscle activation of the patient,
for determining a second piece of information on an actual respiratory muscle activation of the patient, and
for determining an indicator of a breathing assistance of the patient based on the first information and based on the second information.

In a preferred embodiment based on the above-described embodiment, the device comprises a device for airway flow measurement and for airway pressure measurement at the patient. The control unit may be configured here to determine the first information and the second information on the basis of an air flow measurement and of an airway pressure measurement.

In a preferred embodiment based on at least one of the above-described embodiments, the device may further comprise a device for the pneumatic breathing assistance. The indicator of the breathing assistance may comprise an indicator of the pneumatic breathing assistance.

In a preferred embodiment based on at least one of the above-described embodiments, the device may further comprise a device for sedating the patient based on the indicator the breathing assistance.

In a preferred embodiment based on at least one of the above-described embodiments, the device may be configured to obtain measurement information on an airway flow measurement or an airway pressure measurement at the patient via the one or more interfaces. The control unit may be configured in this preferred embodiment to further determine the indicator of the breathing assistance on the basis of the measurement information.

In a preferred embodiment based on at least one of the above-described embodiments, the device may further comprise a device for the sensor-based detection of a signal, which depends on the actual respiratory muscle activation. The control unit may be configured in this preferred embodiment to determine the indicator of the breathing assistance on the basis of the sensor-detected signal.

In a preferred embodiment based on at least one of the above-described embodiments, the device for the sensor-based detection may be configured to detect an electromyogram, a mechanomyogram or an electrical impedance myogram.

In a preferred embodiment based on at least one of the above-described embodiments, the device for the sensor-based detection may comprise a strain sensor, an ultrasound sensor or an esophageal pressure sensor.

In a preferred embodiment based on at least one of the above-described embodiments, the control unit may be configured to reduce a difference between the first information and the second information on the indicator of the breathing assistance by means of a control.

In a preferred embodiment based on at least one of the above-described embodiments, the control unit may be configured to generate a stimulation signal for the patient as an indicator of the breathing assistance.

In a preferred embodiment based on at least one of the above-described embodiments, the device may further comprise a device for the stimulation of the respiratory muscles of the patient on the basis of the indicator of the breathing assistance.

In a preferred embodiment based on at least one of the above-described embodiments, the first information and the second information may comprise each an indicator of a patient-side, stimulated or total respiratory muscle activation, a patient-side, stimulated or total respiratory muscle flow or a patient-side, stimulated or total respiratory muscle pressure.

In a preferred embodiment based on at least one of the above-described embodiments, the control unit may be configured to determine and to provide information on a respiratory muscle activation elicited by spontaneous breathing activity of the patient, on a respiratory muscle flow elicited by spontaneous breathing activity of the patient, or on a respiratory muscle pressure elicited by a spontaneous breathing activity of the patient.

In a preferred embodiment based on at least one of the above-described embodiments, the control unit may be configured to determine the indicator of the breathing assistance on the basis of the information on the spontaneous breathing activity of the patient.

In a preferred embodiment based on at least one of the above-described embodiments, the indicator of the breathing assistance may indicate an indicator of a more intensive sedation of the patient when the information on the spontaneous breathing activity of the patient indicates a respiratory muscle activity above the desired respiratory muscle activation.

In a preferred embodiment based on at least one of the above-described embodiments, the control unit may be configured to carry out an estimation for a stimulation pulse response of the patient on the basis of the second information in response to the indicator of the breathing assistance.

In a preferred embodiment based on at least one of the above-described embodiments, the control unit may be configured to determine the estimation on the basis of a stimulation maneuver.

In a preferred embodiment based on at least one of the above-described embodiments, the control unit may be configured to repeat the estimation at regular intervals or continuously.

In a preferred embodiment based on at least one of the above-described embodiments, the control unit may be configured to determine and to display a respective reliability for the first information and for the second information when the reliability drops below a predefined threshold value.

An especially preferred embodiment based on at least one of the above-described embodiments may be configured as a ventilator.

In a preferred embodiment of the ventilator based on the above-described embodiment, the control unit may be configured to communicate information on the indicator of the breathing assistance via the one or more interfaces.

In a preferred embodiment of the ventilator based on at least one of the above-described embodiments, the indicator of the breathing assistance may comprise at least one parameter of the group comprising
an amplitude,
a ramp slope,
a stimulation duration,
a start time,
an end time,
an actual respiratory muscle activation, and
the desired respiratory muscle activation.

In a preferred embodiment of the ventilator based on at least one of the above-described embodiments, the one or more interfaces may be configured for real-time communication with a stimulator and/or with a sensor unit.

In a preferred embodiment of the ventilator based on at least one of the above-described embodiments, the one or more interfaces may be configured
for real-time communication with a stimulator and/or with a sensor unit,
for the communication of a time course of the indicator for the breathing assistance with a stimulator and/or with a sensor unit.
   The real-time communication may preferably be configured via the one or more interfaces as a sample-by-sample real-time communication, sample by sample meaning that the communication is based on the sequence of sample values, i.e., sample value for sample value.

In a preferred embodiment of the ventilator based on at least one of the above-described embodiments, the control unit may be configured to predefine a cycle for a stimulator via the one or more interfaces.

In an especially preferred embodiment of the ventilator based on at least one of the above-described embodiments, the ventilator may have an integrated stimulator.

In a preferred embodiment of the stimulator based on the above-described embodiment, the control unit may be configured to receive the first information and the second information via the one or more interfaces from a sensor unit or from a ventilator.

In a preferred embodiment of the stimulator based on the above-described embodiment, the control unit may be configured to receive at least one piece of information from the group comprising
an amplitude,
a ramp slope,
a stimulation duration,
a start time,
an end time,
a time course of the desired respiratory muscle activation,
an airway flow,
an airway pressure,
a respiratory muscle action, and
a respiratory muscle flow from a sensor unit via the one or more interfaces.

In a preferred embodiment of the stimulator based on the above-described embodiment, the control unit may be configured to receive a cycle from a sensor unit and/or from a ventilator via the one or more interfaces.

In a preferred embodiment of the stimulator based on the above-described embodiment, the control unit may be configured to predefine a cycle for a sensor unit and/or for a ventilator via the one or more interfaces.

In a preferred embodiment of the stimulator based on the above-described embodiment, the control unit may be configured to communicate in real time via the one or more interfaces.

In a preferred embodiment of the stimulator based on the above-described embodiment, the control unit may be configured to coordinate ventilation maneuvers with a ventilator via the one or more interfaces.

An especially preferred embodiment based on at least one of the above-described embodiments may be configured as a sensor unit.

In a preferred embodiment of the sensor unit based on the above-described embodiment, the control unit may be configured to receive information on at least one pneumatic signal from a ventilator via the one or more interfaces and further to determine the indicator of the breathing assistance on the basis of the information on the at least one pneumatic signal.

In a preferred embodiment of the sensor unit based on the above-described embodiment, the control unit may be configured to determine the second information on the basis of sensor signals.

In a preferred embodiment of the sensor unit based on the above-described embodiment, the control unit may be configured to communicate in real time via the one or more interfaces.

In a preferred embodiment of the sensor unit based on the above-described embodiment, the control unit may be configured to provide at least one piece of information from the group comprising
an amplitude,
a ramp slope,
a stimulation duration,
a start time,
an end time,
an actual respiratory muscle activation,
an airway flow,
an airway pressure,
a respiratory muscle pressure,
a respiratory muscle action, and
a respiratory muscle flow as an indicator of the breathing assistance to a ventilator or to a stimulator via the one or more interfaces.

In a preferred embodiment of the sensor unit based on the above-described embodiment, the control unit may be configured to receive information on a maneuver from a ventilator or from a stimulator via the one or more interfaces.

In a preferred embodiment of the sensor unit based on the above-described embodiment, the control unit may be configured to receive information on
a gating,
measurement times to be blanked out,
a filtering,
a suppression of stimulation artifacts
   as first information from a ventilator or from a stimulator via the one or more interfaces.

An especially preferred embodiment based on at least one of the above-described embodiments may be configured as a ventilation system.

In a preferred embodiment of the ventilation system, based on at least one of the above-described embodiments, the ventilation system may also comprise a stimulator.

In a preferred embodiment of the ventilation system based on at least one of the above-described embodiments, the ventilation system may comprise a sensor unit.

A basic embodiment shows a process for a component of a ventilation system for the breathing assistance of a patient with
determination of a first piece of information on a desired respiratory muscle activation of the patient,
determination of a second piece of information on an actual respiratory muscle activation of the patient, and
determination of an indicator of a breathing assistance of the patient based on the first information and based on the second information.

A preferred embodiment of the process based on the above-described embodiment may comprise the determination of the first information and of the second information based on an airway flow measurement and of an airway pressure measurement.

In a preferred embodiment of the process based on at least one of the above-described embodiments, the indicator of the breathing assistance may comprise an indicator of the pneumatic breathing assistance.

A preferred embodiment of the process based on at least one of the above-described embodiments may comprise a sedation of the patient based on the indicator of the breathing assistance.

A preferred embodiment of the process based on at least one of the above-described embodiments may comprise the obtaining of information on an airflow measurement or on an airway pressure measurement at the patient and it may comprise a determination of the indicator of the breathing assistance based on the measurement information.

A preferred embodiment of the process based on at least one of the above-described embodiments may comprise a sensor-based detection of a signal, which depends on the actual respiratory muscle activation, and it may comprise a determination of the indicator of the breathing assistance based on the sensor-detected signal.

In a preferred embodiment of the process based on the above-described embodiment, the sensor-detected signal may comprise an electromyogram, a mechanomyogram, or an electrical impedance myogram.

In a preferred embodiment of the process based on at least one of the above-described embodiments, the detection of the sensor signal may be carried out with a strain sensor, with an ultrasound sensor or with an esophageal pressure sensor.

A preferred embodiment of the process based on at least one of the above-described embodiments may comprise a stimulation of the respiratory muscles of the patient based on the indicator of the breathing assistance.

A preferred embodiment of the process based on at least one of the above-described embodiments may comprise a reduction of a difference between the first information and the second information by control by means of the indicator of the breathing assistance of the patient.

A preferred embodiment of the process based on at least one of the above-described embodiments may comprise the generation of a stimulation signal for the patient as an indicator of the breathing assistance.

In a preferred embodiment of the process based on at least one of the above-described embodiments, the first information and the second information may comprise each an indicator of a patient-side, stimulated or total respiratory muscle activation, a patient-side, stimulated or total respiratory muscle flow or a patient-side, stimulated or total respiratory muscle pressure.

A preferred embodiment of the process based on at least one of the above-described embodiments may comprise a determination and provision of information on a respiratory muscle activation elicited by spontaneous breathing activity of the patient, a respiratory muscle flow elicited by spontaneous breathing activity of the patient or a respiratory muscle pressure elicited by spontaneous breathing activity of the patient.

In a preferred embodiment of the process based on at least one of the above-described embodiments, the indicator of the breathing assistance may indicate an indicator of a more intensive sedation of the patient when the information on the spontaneous breathing activity of the patient indicates a respiratory muscle activity above the desired respiratory muscle activity.

A preferred embodiment of the process based on at least one of the above-described embodiments may comprise an estimation of a stimulation pulse response of the patient from the second information in response to the indicator of the breathing assistance.

In a preferred embodiment of the process based on at least one of the above-described embodiments, the estimation may further take place on the basis of a stimulation maneuver.

In a preferred embodiment of the process based on at least one of the above-described embodiments, the estimation may be carried out at regular intervals or continuously.

A preferred embodiment of the process based on at least one of the above-described embodiments may comprise a determination—or also an output and/or a display—of an additional piece of information, for example, reliability, in addition to the first information and to the second information, when the reliability drops below a predefined threshold.

A preferred embodiment of the process based on at least one of the above-described embodiments may comprise a process for a ventilator.

A preferred embodiment of the process based on the above-described embodiment may comprise a communication of information on the indicator of the breathing assistance to a stimulator.

In a preferred embodiment of the process based on the above-described embodiment, the indicator of the breathing assistance may comprise at least one parameter of the group comprising an amplitude, a ramp slope, a stimulation duration, a start time, an actual respiratory muscle activation, an end time and the desired respiratory muscle activation.

In a preferred embodiment of the process based on at least one of the above-described embodiments, the communication with a stimulator and/or with a sensor unit may take place in real time.

In a preferred embodiment of the process based on at least one of the above-described embodiments, the communication may take place “sample by sample,” i.e., sample value by sample value, oriented on the sequence of sample values, in real time with a stimulator and/or with a sensor unit.

In a preferred embodiment of the process based on at least one of the above-described embodiments, the communication may comprise communication of a time course of the indicator of the breathing assistance with a stimulator and/or with a sensor unit.

In a preferred embodiment of the process based on at least one of the above-described embodiments, the communication may comprise the predefinition of a cycle to a stimulator.

A preferred embodiment of the process based on at least one of the above-described embodiments may comprise predefinition of the first information and of the second information to a stimulator and/or coordination of a ventilation maneuver with a stimulator.

A preferred embodiment of the process based on at least one of the above-described embodiments may comprise a process for a stimulator.

A preferred embodiment of the process based on at least one of the above-described embodiments may comprise receipt of the first information and of the second information from a sensor unit or from a ventilator.

A preferred embodiment of the process based on at least one of the above-described embodiments may comprise the receipt from a sensor unit of at least one piece of information from the group comprising
an amplitude,
a ramp slope,
a stimulation duration,
a start time,
an end time,
an actual respiratory muscle activation,
the desired respiratory muscle activation,
an airway flow,
an airway pressure,
a respiratory muscle pressure,
a respiratory muscle action, and
a respiratory muscle flow.

A preferred embodiment of the process based on at least one of the above-described embodiments may comprise predefinition of a cycle for a sensor unit and/or for a ventilator.

A preferred embodiment of the process based on at least one of the above-described embodiments may comprise a communication in real time.

A preferred embodiment of the process based on at least one of the above-described embodiments may comprise coordination of a ventilation maneuver with a ventilator.

A preferred embodiment of the process based on at least one of the above-described embodiments may comprise a process for a sensor unit.

A preferred embodiment of the process based on at least one of the above-described embodiments may comprise the receipt of information on at least one pneumatic signal from a ventilator and determination of the indicator of the breathing assistance based on the information on the at least one pneumatic signal.

A preferred embodiment of the process based on at least one of the above-described embodiments may comprise a determination of the second information based on sensor signals.

A preferred embodiment of the process based on at least one of the above-described embodiments may comprise a communication in real time.

A preferred embodiment of the process based on at least one of the above-described embodiments may comprise the provision of at least one piece of information from the group comprising an amplitude, a ramp slope, a stimulation duration, a start time, an end time, an actual respiratory muscle activation, an airway flow, an airway pressure, a respiratory muscle pressure, a respiratory muscle action and a respiratory muscle flow as an indicator of the breathing assistance to a ventilator or to a stimulator.

A preferred embodiment of the process based on at least one of the above-described embodiments may comprise the obtaining of information on a maneuver.

In a preferred embodiment of the process based on at least one of the above-described embodiments, the receipt as first information from a ventilator or from a stimulator may comprise information on
a gating,
measurement times to be blanked out,
a filtering, and
a suppression of stimulation artifacts.

A preferred embodiment of the process based on at least one of the above-described embodiments may comprise a process
for a ventilation system,
for a ventilator,
for a stimulator and
for a sensor unit.

A basic embodiment may comprise a computer program with a program code for carrying out at least one of the above-described embodiments. The program code may be advantageously executed on a computer, on a processor or on a programmable hardware component.

Additional and preferred embodiments of the stimulation device according to the present invention will be described below concerning a ventilation system, a device, a process and a computer program for the stimulative ventilatory assistance of a patient, especially concerning a concept for the stimulative ventilatory assistance of a patient, synchronized with a spontaneously generated respiratory muscle activity of the patient. A basic embodiment of the stimulation device shows a stimulation device for the stimulative ventilatory assistance of a patient with one or more interfaces for communication, with components of the ventilation system and with a control unit, which is configured for the detection of information on a time course of an activation signal of the respiratory muscles of the patient.

In a preferred embodiment of the stimulation device based on the above-described embodiment of the stimulation device, the control unit may be configured to carry out
detection of information on a time course of a component of the work of breathing, which is contributed by the patient themself and
determination of the activation signal for the respiratory muscles of the patient on the basis of the information on the time course of the component contributed by the patient themself to the work of breathing
   for detecting the information on the time course of the activation signal.

In a preferred embodiment of the stimulation device based on at least one of the above-described embodiments of the stimulation device the control unit may be configured to determine and to take into consideration a lower activation threshold for the stimulation. In this preferred embodiment of the stimulation device
activation of the respiratory muscles can take place in case of a stimulation of the respiratory muscles above the activation threshold and
activation can be at least reduced or omitted in case of a stimulation of the respiratory muscles below the activation threshold.

In a preferred embodiment of the stimulation device based on at least one of the above-described embodiments of the stimulation device, the control unit may be configured to carry out the stimulation in a positive feedback with the activation signal.

In a preferred embodiment of the stimulation device based on at least one of the above-described embodiments of the stimulation device, the control unit may be configured to determine a stimulating effect.

In a preferred embodiment of the stimulation device based on the above-described embodiment of the stimulation device, the control unit may be configured to carry out a titration for determining the stimulating effect.

In a preferred embodiment of the stimulation device based on at least one of the above-described embodiments of the stimulation device, the control unit may be configured to carry out the stimulation proportionally to the activation signal.

In a preferred embodiment of the stimulation device based on at least one of the above-described embodiments of the stimulation device, the control unit may be configured to determine information on at least one element from the group comprising
a muscle pressure Pₘᵤₛ,
a spontaneous muscle pressure Pₛₚₒₙ,
a breathing gas flow Flowₘᵤₛ caused by the muscles of the patient,
a breathing gas flow Flowₛₚₒₙ caused spontaneously by the muscles of the patient,
a work of breathing WOB contributed by the patient themself, and
a work of breathing WOBₛₚₒₙ contributed spontaneously by the patient themself
   as information on the time course of the activation signal of the respiratory muscles of the patient.

In a preferred embodiment of the stimulation device based on at least one of the above-described embodiments of the stimulation device, the control unit may be configured to ventilate the patient simultaneously pneumatically.

In a preferred embodiment of the stimulation device based on the above-described embodiment of the stimulation device, the control unit may be configured to carry out the simultaneous pneumatic ventilation as proportional ventilation.

In a preferred embodiment of the stimulation device based on at least one of the above-described embodiments of the stimulation device, the control unit may be configured to carry out the detection of the information on the time course of the activation signal of the respiratory muscles of the patient on the basis of an electromyographic signal, of a signal from an electrical impedance myography, of a signal of a strain sensor, of a signal of an ultrasound sensor or of a mechanomyographic sensor.

In a preferred embodiment of the stimulation device based on at least one of the above-described embodiments of the stimulation device, the control unit may be configured to set an intensity of the stimulation based on a predefined degree.

In a preferred embodiment of the stimulation device based on the above-described embodiment of the stimulation device, the control unit may be configured to determine the predefined degree by means of a ratio of the stimulation intensity to the activation signal or of the stimulation intensity to the estimated breathing effort Pₘᵤₛ.

In a preferred embodiment of the stimulation device based on the above-described embodiment of the stimulation device, the degree may be set as a ratio of a stimulated breathing activity to a spontaneous breathing activity of the patient.

In a preferred embodiment of the stimulation device based on the above-described embodiment of the stimulation device, the control unit may be configured to determine the spontaneous breathing activity of the patient on the basis of the information on the time course of the activation signal of the respiratory muscles of the patient.

In a preferred embodiment of the stimulation device based on at least one of the above-described embodiments of the stimulation device, the control unit may be configured to determine a stimulation signal based on the ratio of the stimulated breathing activity to the spontaneous breathing activity of the patient and to an activation pulse response of the muscles of the patient.

In a preferred embodiment of the stimulation device based on at least one of the above-described embodiments, the control unit may be configured to determine the stimulation signal by deconvolution of a desired activation signal EMGₛₜᵢₘ elicited by stimulation with the activation pulse response.

In a preferred embodiment of the stimulation device based on at least one of the above-described embodiments, the control unit may be configured to determine the spontaneous breathing activity as a respiratory muscle pressure Pₛₚₒₙ generated spontaneously by the patient and the stimulated breathing activity as a respiratory muscle pressure Pₛₜᵢₘ generated by stimulation.

In a preferred embodiment of the stimulation device based on at least one of the above-described embodiments, the control unit may be configured to determine the spontaneous breathing activity as a breathing gas flow Flowₛₚₒₙ generated spontaneously by the patient and the stimulated breathing activity as a breathing gas flow Flowₛₜᵢₘ generated by stimulation.

In a preferred embodiment of the stimulation device based on at least one of the above-described embodiments, the control unit may be configured to determine
the spontaneous breathing activity as a work of breathing WOBₛₚₒₙ generated spontaneously by the patient
or a time derivative dWOBₛₚₒₙ/dt thereof
and the stimulated breathing activity as a work of breathing WOBₛₜᵢₘ generated by stimulation
or the time derivative dWOBₛₜᵢₘ/dt thereof.

In a preferred embodiment of the stimulation device based on at least one of the above-described embodiments, the control unit may be configured to adjust the predefined degree on a time plane, which is greater than or equal to a breathing cycle of the patient.

In a preferred embodiment of the stimulation device based on at least one of the above-described embodiments, the control unit may be configured to control the stimulation for reaching the target value, which comprises
a patient-side component of a driving pressure ΔPₘᵤₛ,
a patient-side component of a volume ΔVₘᵤₛ, and
a patient-side component of a work of breathing ΔWOBₘᵤₛ.

An embodiment based on at least one of the above-described embodiments of the stimulation device shows a ventilation system with a stimulation device for a stimulative ventilatory assistance of a patient.

A basic embodiment of the stimulation device shows a process for a stimulative ventilatory assistance of a patient with
a detection of information on a time course of an activation signal of the respiratory muscles of the patient and
a stimulation of the respiratory muscles in a chronological alignment with the activation signal for the muscular ventilatory assistance of the patient.

In a preferred embodiment of the process for the stimulative ventilatory assistance of a patient based on at least one of the above-described embodiments, the detection of the information on the time course of the activation signal may comprise
a detection of information on a time course of a component contributed by the patient themself to the work of breathing
   and
a determination of the activation signal for the respiratory muscles of the patient based on the information on the time course of the component contributed by the patient themself to the work of breathing.

A preferred embodiment of the process for the stimulative ventilatory assistance of a patient based on at least one of the above-described embodiments may comprise the determination and the taking into consideration of a lower activation threshold for the stimulation, wherein
activation of the respiratory muscles takes place during a stimulation of the respiratory muscles above the activation threshold and
activation is at least reduced or omitted in case of a stimulation of the respiratory muscles below the activation threshold.

In a preferred embodiment of the process for the stimulative ventilatory assistance of a patient based on at least one of the above-described embodiments, the stimulation can take place in a positive feedback with the activation signal.

A preferred embodiment of the process for the stimulative ventilatory assistance of a patient based on at least one of the above-described embodiments may comprise determination of a stimulating effect.

A preferred embodiment of the process for the stimulative ventilatory assistance of a patient based on the above-described embodiment may comprise a titration for determining the stimulating effect.

In a preferred embodiment of the process for the stimulative ventilatory assistance of a patient based on at least one of the above-described embodiments, the stimulation may take place proportionally to the activation signal.

In a preferred embodiment of the process for the stimulative ventilatory assistance of a patient based on at least one of the above-described embodiments, the information on the time course of the activation signal of the respiratory muscles of the patient may comprise information on at least one element from the group comprising
a muscle pressure Pₘᵤₛ,
a spontaneous muscle pressure Pₛₚₒₙ,
a breathing gas flow Flowₘᵤₛ caused by the muscles of the patient,
a breathing gas flow Flowₛₚₒₙ caused spontaneously by the muscles of the patient,
a work of breathing WOB performed by the patient themself, and
a spontaneous work of breathing WOBₛₚₒₙ contributed spontaneously by the patient themself.

A preferred embodiment of the process for the stimulative ventilatory assistance of a patient based on at least one of the above-described embodiments may comprise a simultaneous pneumatic ventilation of the patient.

In a preferred embodiment of the process for the stimulative ventilatory assistance of a patient based on at least one of the above-described embodiments, the simultaneous pneumatic ventilation may comprise a proportional ventilation.

In a preferred embodiment of the process for the stimulative ventilatory assistance of a patient based on at least one of the above-described embodiments, the detection of the information on the time course of the activation signal of the respiratory muscles of the patient may be carried out on the basis of
an electromyographic signal,
a signal from an electrical impedance myography,
a signal of a strain sensor,
a signal of an ultrasound sensor,
a mechanomyographic signal.

A preferred embodiment of the process for the stimulative ventilatory assistance of a patient based on at least one of the above-described embodiments may comprise a setting of an intensity of the stimulation based on a predefined degree.

In a preferred embodiment of the process for the stimulative ventilatory assistance of a patient based on the above-described embodiment, the predefined degree may comprise a ratio of a stimulation intensity and activation signal or of a stimulation intensity and estimated breathing effort Pₘᵤₛ.

In a preferred embodiment of the process for the stimulative ventilatory assistance of a patient based on the above-described embodiment, the degree may set a ratio of a stimulated breathing activity to a spontaneous breathing activity of the patient.

A preferred embodiment of the process for the stimulative ventilatory assistance of a patient based on the above-described embodiment may comprise a determination of the spontaneous breathing activity of the patient based on the information on the time course of the activation signal of the respiratory muscles of the patient.

In a preferred embodiment of the process for the stimulative ventilatory assistance of a patient based on at least one of the above-described embodiments may comprise a determination of a stimulation signal based on the ratio of the stimulated breathing activity and the spontaneous breathing activity of the patient and an activation pulse response of the muscles of the patient.

A preferred embodiment of the process for the stimulative ventilatory assistance of a patient based on the above-described embodiment may comprise the determination of the stimulation signal by deconvolution of a desired activation signal EMGₛₜᵢₘ elicited by stimulation with the activation pulse response.

In a preferred embodiment of the process for the stimulative ventilatory assistance of a patient based on at least one of the above-described embodiments, the spontaneous breathing activity may comprise a respiratory muscle pressure Pₛₚₒₙ generated spontaneously by the patient and the stimulated breathing activity may comprise a respiratory muscle pressure Pₛₜᵢₘ generated by stimulation.

In a preferred embodiment of the process for the stimulative ventilatory assistance of a patient based on at least one of the above-described embodiments
the spontaneous breathing activity may comprise a breathing gas flow Flowₛₚₒₙ generated spontaneously by the patient
   and
the stimulated breathing activity may comprise a breathing gas flow Flowₛₜᵢₘ generated by the stimulation.

In a preferred embodiment of the process for the stimulative ventilatory assistance of a patient based on at least one of the above-described embodiments,
the spontaneous breathing activity may comprise a work of breathing WOBₛₚₒₙ generated spontaneously by the patient
or a time derivative dWOBₛₚₒₙ/dt thereof.
   and
the stimulated breathing activity may comprise a work of breathing WOBₛₜᵢₘ generated by stimulation
or the time derivative dWOBₛₜᵢₘ/dt thereof.

A preferred embodiment of the process for the stimulative ventilatory assistance of a patient based on at least one of the above-described embodiments may comprise an adjustment of the predefined degree on a time plane that is greater than or equal to a breathing cycle of the patient.

In a preferred embodiment of the process for the stimulative ventilatory assistance of a patient based on at least one of the above-described embodiments, control of the stimulation for reaching a target value may comprise
a patient-side component of a driving pressure ΔPₘᵤₛ,
a patient-side component of a volume ΔVₘᵤₛ,
or a patient-side component of a work of breathing ΔWOBₘᵤₛ.

A basic embodiment may comprise a computer program with a program code for carrying out at least one of the above-described embodiments of the stimulation device. The program code may advantageously be executed on a computer, on a processor or on a programmable hardware component.

Table 3 below comprises the abbreviations and designations used within the framework of the present invention, associated with respective brief explanations.

TABLE 3

| Symbol | Description/brief explanation | Units |
|---|---|---|
| R | Resistance of the respiratory system; R = P/(dV/dt) | mbar/L/sec) |
| C | Compliance; C = V/P | L/mbar |
| E | Elasticity of the respiratory system; E = 1/C; E = P/V | mbar/L |
| τ, tau | Time constant of the respiratory system | sec |
| s | Frequency (complex number) | sec⁻¹ |
| Flow, dV/dt | Breathing gas flow, flow rate of gases that flow away from the | L/min; |
| | patient or towards the patient | L/sec |
| Vol, V | Volume, which flows from/to the patient | L |
| sEMG, | EMG signal, time course of the EMG signal | μV |
| sEMG(t) | | |
| MV | Minute volume | L |
| V_{T} | Tidal volume of the patient | L |
| Vol′_{ges} | Total volume flow flowing from/to the patient | L/min, |
| | | L/sec |
| Volₘᵤₛ | Volume caused by muscle activity | L |
| Vol_{musMax} | Maximum volume caused by muscle activity | L |
| Vol′ₘᵤₛ | Volume flow caused by muscle activity | L/sec |
| Flowₛₚₒₙ | Breathing gas flow caused by muscular spontaneous activity | L/min, |
| | | L/sec |
| Flowₘᵤₛ | Volume flow caused by muscle activity | L/min, |
| | | L/sec |
| Volᵥₑₙₜ | Volume generated by the ventilator | L |
| Vol′ᵥₑₙₜ | Breathing gas flow generated by the ventilator | L/min, |
| | | L/sec |
| Volₛₜᵢₘ | Volume brought about by stimulation | L |
| V_{T mus} | Tidal volume generated by muscle activity | L |
| MVₘᵤₛ | Minute volume generated by muscle activity | L |
| P_{aw} | Airway pressure | mbar |
| P_{drv} | Total pressure acting on the respiratory system (driving | mbar |
| | pressure) | |
| Pᵥₑₙₜ | Ventilation pressure, pressure generated by the ventilator | mbar |
| Pₘᵤₛ, Pₘᵤₛ(t) | Pressure caused by muscle activity | mbar |
| P_{mus base}, | Basic breathing load, pressure needed at least to overcome the | mbar |
| P_{base} | respiratory resistances | |
| P_{musMax} | Pressure caused by maximum muscle activity | mbar |
| PIₘₐₓ | Maximum pressure generated during inhalation during mouth | mbar |
| | closing | |
| Pₛₚₒₙ | Spontaneous muscle pressure, pressure caused by spontaneous | mbar |
| | muscle activity | |
| Pₛₜᵢₘ | Stimulated muscle pressure, pressure caused by muscle | mbar |
| | stimulation | |
| P_{insp.} | Airway pressure during the phase of inhalation (inhalation) | mbar |
| P_{exp.} | Airway pressure during the phase of exhalation (exhalation) | mbar |
| PEEP | Positive end-expiratory pressure at the end of the phase of | mbar |
| | exhalation | |
| iPEEP | Intrinsic end-expiratory pressure | mbar |
| WOB | Work of breathing, | Nm, J |
| | [mbar * I] = 1 hPa*m³/1000] = 0.1 Nm | |
| WOBₜₒₜ | Total work of breathing | Nm, J |
| WOBᵥₑₙₜ | Work of breathing generated by the ventilator | Nm, J |
| WOBₛₚₒₙ | Work of breathing generated by spontaneous muscle activity | Nm, J |
| WOBₛₜᵢₘ | Work of breathing generated by stimulation | Nm, J |
| WOB_{base} | Basic breathing load in the sense of the work of breathing | Nm, J |
| WOB_{musMax} | Work of breathing generated by maximum muscle activity | Nm, J |
| NVE | Neuroventilatory efficiency | L/μV |
| NME | Neuromechanical efficiency | mbar/μV |
| LBC | Load bearing capacity | — |
| LI | Load (Load Index) | — |
| EMGₛₜᵢₘ | EMG signal generated by stimulation | μV |
| EMGₛₚₒₙ | EMG signal generated by spontaneous muscle activity | μV |
| SVE(t) | Ventilatory efficiency relative to muscle stimulation | I/% |
| | EVE(t) k(t) · NVEstim | |
| k(t) | Activatability | μV/% |
| NVEₛₜᵢₘ | Neuroventilatory efficiency during stimulation | L/μV |
| NMEₛₜᵢₘ | Neuromechanical efficiency during stimulation | mbar/μV |
| NMEₛₚₒₙ | Neuromechanical efficiency during spontaneous breathing/ | mbar/μV |
| | spontaneous muscle activity | |
| NVEₛₚₒₙ | Neuroventilatory efficiency during spontaneous breathing/ | L/uV |
| | spontaneous muscle activity | |
| Iₛₜᵢₘ, Iₛₜᵢₘ(t) | Stimulation intensity, intensity of the stimulation signal | % |
| a2 | Component of driving pressure | — |
| b2 | Component of driving pressure | — |
| α | Positive feedback factor | — |
| β | β= α/(1 + α) | — |
| intercost.stm | “intercost stm” signal = stimulation signal, binary (0/1) | |
| costmar.org | “costmar.org” signal = original EMG signal derived at the | μV |
| | costal margin by means of two electrodes | |
| costmar.ecgr | ecgr signal = “costmar.org” signal with offset & stimulation | μV |
| | artifacts removed | |
| costmar.env | “costmar.env” signal = envelope of the “costmar.egcr” [sic - | μV |
| | Tr.Ed.] signal | |
| costmar.avg | “costmar.avg” signal = (long-term) average of the | μV |
| | “costmar.org” signal | |

All the patent documents and publications along with publication numbers and with short titles of the publications are listed in Table 4 below. The full titles can be found in the explanations on the state of the art in the introduction to the specification. The reference numbers [E1] through [E38] listed in Table 4 are used at times in the specification.

TABLE 4

| [Ref] | Publication/disclosure/brief title |
|---|---|
| [E1] | US5820560 |
| [E5] | WO2019154834 |
| [E6] | WO2019154837 |
| [E7] | WO2019154839 |
| [E8] | WO2020079266 |
| [E9] | DE102019006480 |
| [E11] | Walker, D. J.: “Prediction of the esophageal |
| | pressure . . . ” |
| [E13] | US2017/0252558 |
| [E14] | DE102019006480 |
| [E15] | DE102019007717 |
| [E16] | DE102020000014 |
| [E17] | DE102007062214 |
| [E18] | WO2018143844 |
| [E19] | Kahl, L. et al.: “Comparison of algorithms to . . . ” |
| [E20] | DE102015011390 |
| [E21] | Jansen D. et al.: “Estimation of the diaphragm |
| | neuromuscular . . . ” |
| [E22] | Liu L. et al.: “Neuroventilatory efficiency and extubation |
| | readiness . . . ” |
| [E23] | Cattapan, S. E. et al.: “Can diaphragmatic contractility |
| | be assessed by . . . ” |
| [E24] | Younes, M. et al.: “A method for monitoring and |
| | improving . . . ” |
| [E25] | Putensen C. et al.: “Long-term effects of spontaneous |
| | breathing . . . ” |
| [E26] | Bellani, G. et al.:” “The Ratio of Inspiratory |
| | Pressure Over . . . ” |
| [E27] | US7021310 |
| [E28] | US20120103334 |
| [E29] | DE102021115865 |
| [E30] | Otis, A. B. et al.: “Mechanics of Breathing in Man” |
| [E31] | US20150366480 |
| [E32] | WO2020188069 |
| [E34] | DE102020123138 |
| [E35] | Shalaby, R. E .-S.: “Development of an Electromyography |
| | Detection System . . . ” |
| [E36] | Osuagwu, B. A. C. et al.: “Active proportional electromyogram |
| | controlled . . . ” |
| [E37] | DE102007052897 |
| [E38] | Virtanen, J. et al.: “Instrumentation for the measurement of |
| | electric . . . ” |

1. A device for determining a state of respiratory muscles of a patient, the device comprising:
an interface arrangement comprising one or more interfaces, the interface arrangement being configured to detect patient signals; and
a control unit configured to:
stimulate the respiratory muscles of the patient with a stimulation signal;
detect an activation signal as a response to the stimulation; and
determine one or more state parameters for the respiratory muscles based on the stimulation signal and on the activation signal.

2. The device in accordance with claim 1, wherein the control unit is configured to generate the stimulation signal with one or more stimulation pulses.

3. The device in accordance with claim 1, wherein the control unit is configured to detect the activation signal as a pulse response.

4. The device in accordance with claim 1, wherein the control unit is configured to determine an activatability of the respiratory muscles of the patient with the determination of the one or more state parameters.

5. The device in accordance with claim 4, wherein the control unit is configured to take into consideration an activation threshold for the stimulation signal during the determination of the activatability, wherein an activation of the respiratory muscles takes place during a stimulation of the respiratory muscles above the activation threshold and an activation is at least reduced or is not performed in case of a stimulation of the respiratory muscles below the activation threshold.

6. The device in accordance with claim 5, wherein the control unit is configured to determine at least one of:
a respiratory muscle pressure, which can be generated by stimulation;
a tidal volume, which can be generated by stimulation; and
a work of breathing of the patient, which can be generated by stimulation.

7. The device in accordance with claim 1, wherein the control unit is further configured to signal the interface arrangement to perform a pneumatic diagnostic maneuver for determining a pneumatic ventilation parameter and further to determine the one or more state parameters based on the pneumatic ventilation parameter.

8. The device in accordance with claim 7, wherein the pneumatic diagnostic maneuver comprises one or more of:
an occlusion;
a breath flow limitation;
an omission of an assistance of individual breaths; and
a variability in the breathing assistance of the patient.

9. The device in accordance with claim 1 wherein the control unit is further configured to determine an indicator of a maximum possible breathing effort of the patient.

10. The device in accordance with claim 9, wherein the indicator of the maximum possible breathing effort of the patient comprises a mouth closing pressure at maximum activation of the respiratory muscles.

11. The device in accordance with claim 1, wherein the control unit is configured to determine one or more of an indicator of a load-bearing capacity of the respiratory muscles of the patient and an indicator of an efficiency of the respiratory muscles of the patient.

12. The device in accordance with claim 11, wherein at least one of:
the indicator of the load-bearing capacity is based on a relationship between a basic load, and a maximum possible breathing effort, of the patient; and
the indicator of the efficiency comprises a ratio of a tidal volume achievable by stimulation or respiratory muscle pressure to the activation signal.

13. The device in accordance with claim 1, wherein the control unit is configured to output information on the one or more state parameters via the one or more interfaces.

14-25. (canceled)

26. A ventilation system for assisting a patient during the ventilation with a device according to claim 1, further comprising:
one or more interfaces of the interface arrangement, which are configured for an exchange of information with one or more of a ventilating unit, a stimulation unit and a sensor unit; and
wherein the control unit is configured:
to detect an indicator of a component of the ventilation that is contributed by the patient's own efforts;
to determine an indicator of a load-bearing capacity of the patient;
to influence the component contributed by the patient's own efforts and of the ventilation; and
to assist the patient during the ventilation based on the indicator of the component of the ventilation that is contributed by the patient's own efforts himself and based on the indicator of the load-bearing capacity of the patient.

27. The device according to claim 1, wherein the control unit is configured;
to determine a first piece of information on a desired respiratory muscle activation of the patient;
to determine a second piece of information by means of an actual respiratory muscle activation of the patient; and
to determine an indicator of a breathing assistance of the patient based on the first information and based on the second information.

28-39. (canceled)

40. The device according to claim 1, the device further comprising:
one or more interfaces of the interface arrangement, which are configured for an exchange of information with a ventilation unit and with a sensor unit; and
wherein the control unit is configured;
to detect information on a time course of an activation signal of the respiratory muscles of the patient;
to stimulate the respiratory muscles in a chronological alignment with the activation signal for the muscular ventilatory assistance of the patient.

41-52. (canceled)

53. A process for determining a state of respiratory muscles of a patient, the process comprising the steps of:
stimulating the respiratory muscles of the patient with a stimulation signal;
detecting an activation signal as a response to the stimulation; and
determining one or more state parameters for the respiratory muscles based on the stimulation signal and on the activation signal.

54. A process according to claim 53 for ventilating a patient, the process further comprising the steps of:
detecting an indicator of a component contributed by the patient themself to the ventilation;
determining an indicator of a load-bearing capacity of the patient;
influencing the component contributed by the patient themself and of the ventilation; and
assisting the patient during the ventilation based on the indicator of the component contributed by the patient themself to the ventilation and based on the indicator of the load-bearing capacity of the patient.

55-57. (canceled)
